(19) United States
(12) Patent Application Publication
(10) Pub. No.: US 2023/0420077 A1
(43) Pub. Date: Dec. 28, 2023

(54) DISEASE-ASSOCIATED ISOFORM IDENTIFIER

(71) Applicant: Kiromic BioPharma, Inc., Houston, TX (US)

(72) Inventors: Leonardo Mirandola, Plano, TX (US); Maurizio Chiriva-Internati, Manvel, TX (US); Lucia Piccotti, Houston, TX (US); Michael Ryan, Arlington, VA (US)

(21) Appl. No.: 18/037,756

(22) PCT Filed: Nov. 8, 2021
(86) PCT No.: PCT/US21/58436

§ 371 Date: May 18, 2023

Related U.S. Application Data
(60) Provisional application No. 63/115,356, filed on Nov. 18, 2020.

(51) Int. Cl. G16B 25/10; G16B 20/40; G16B 50/00

(52) U.S. Cl. CPC G16B 25/10; G16B 50/00; G16B 20/40

(57) ABSTRACT

The technology relates in part to methods and systems for the identification of disease-associated transcript isoforms and peptides. In certain aspects, the technology relates to methods and systems for the identification of transcript isoforms and peptides preferentially expressed in tumor cells.

### RELATED PATENT APPLICATION

This patent application claims the benefit of U.S. provisional patent application No. 63/115,356 filed on Nov. 18, 2020, naming Leonardo Mirandola et al. as inventors, and designated by attorney docket number KIR-1003-PV. The entire content of the foregoing patent application is incorporated herein by reference for all purposes, including all text, tables and drawings.

### FIELD

The technology relates in part to methods and systems for the identification of disease-associated transcript isoforms and peptides. In certain aspects, the technology relates to methods and systems for the identification of transcript isoforms and peptides preferentially expressed in tumor cells.

### BACKGROUND

Gene expression is the process by which instructions in our DNA are converted into a functional product, such as a protein. One regulatory process of gene expression is alternative splicing, which is a process by which exons of a gene are included or excluded in matured mRNAs, resulting in the production of multiple distinct transcript isoforms, and generating diverse isoforms of proteins. Mechanisms for alternative splicing may include, for example, i) use of alternative promoters, ii) exon skipping, iii) mutually exclusive exons, iv) exon scrambling, v) alternative 5′ and 3′ splice sites, vi) retained introns, and vii) alternative polyadenylation. Aberrant splicing patterns may be found in abnormal cells (e.g., tumor cells). In some instances, dysregulated expression of splicing regulators such as RBFOX2, PTB/PTBP1, and SRSF1, can cause splicing pattern changes in multiple genes.

The advent of whole transcriptome sequencing (RNA-seq) and the development of related bioinformatics analysis tools have enabled researchers to detect and measure not only the expression of genes but also their sequences and structural configurations. When applied to cancer, disease-specific formation of alternative transcripts may be identified as potential biomarkers for diagnosis and/or targets for treatment.

### SUMMARY

Provided in certain aspects are computer-implemented methods for identifying a transcript of a gene that is expressed at a level in a defined subpopulation of diseased tissue samples higher than a level the transcript is expressed in non-diseased tissue samples, comprising (a) receiving user input comprising (i) a defined disease selected from a plurality of defined diseases, where each of the defined diseases corresponds to diseased tissue samples, (ii) a defined minimum transcript expression value threshold for diseased tissue samples, (iii) a defined maximum or a defined median transcript expression value threshold for non-diseased tissues, where each of the non-diseased tissues corresponds to non-diseased tissue samples, and (iv) a defined minimum sample sub-population percentage; (b) identifying in a database comprising one or more tables that relate: (i) transcript identifiers to genes, where at least a portion of the transcript identifiers correspond to transcript isoform sets, and each of the transcript isoform sets is encoded by a gene; (ii) transcript identifiers to corresponding transcript expression values in diseased tissue samples, and (iii) transcript identifiers to corresponding transcript expression values in non-diseased tissue samples; one or more transcript identifiers meeting the following criteria (1) and (2): (1) a corresponding transcript expression value in diseased tissue samples corresponding to the input defined disease of (a)(i) is greater than the input defined minimum transcript expression value threshold of (a)(ii), and (2) a corresponding transcript expression value in non-diseased tissue is less than the input defined maximum or defined median transcript expression value threshold of (a)(iii), for a percentage of diseased samples corresponding to the input defined disease of (a)(i) greater than the defined minimum sub-population percentage of (a)(iv); and (c) outputting a list comprising a transcript identifier for each of one or more transcripts identified in (b), thereby identifying a transcript of one or more genes that is expressed at a level in a defined subpopulation of diseased tissue samples higher than a level the transcript is expressed in non-diseased tissue samples.

Also provided in certain aspects are methods for identifying a transcript of a gene that is expressed at a level in a defined subpopulation of diseased tissue samples higher than a level the transcript is expressed in non-diseased tissue samples, comprising (a) receiving user input comprising (i) a defined disease selected from a plurality of defined diseases, where each of the defined diseases corresponds to diseased tissue samples, (ii) a defined transcript expression ratio threshold, where the transcript expression ratio is a ratio of a transcript expression value for diseased tissue samples to a transcript expression value for non-diseased tissues, where each of the non-diseased tissues corresponds to non-diseased tissue samples, and (iii) a defined minimum sample sub-population percentage; (b) identifying in a database comprising one or more tables that relate (i) transcript identifiers to genes, where at least a portion of the transcript identifiers correspond to transcript isoform sets, and each of the transcript isoform sets is encoded by a gene; and (ii) transcript identifiers to corresponding transcript expression ratios; one or more transcript identifiers having a corresponding transcript expression ratio for the input defined disease of (a)(i) that is greater than the input defined transcript expression ratio threshold of (a)(ii), for a percentage of diseased samples corresponding to the input defined disease of (a)(i) greater than the defined minimum sub-population percentage of (a)(iii); and (c) outputting a list comprising a transcript identifier for each of one or more transcripts identified in (b), thereby identifying a transcript of one or more genes that is expressed at a level in a defined subpopulation of diseased tissue samples higher than a level the transcript is expressed in non-diseased tissue samples.

Also provided in certain aspects are systems, machines, or apparatuses comprising one or more microprocessors and memory, the memory comprising a database comprising one or more tables that relate (i) transcript identifiers to genes, where at least a portion of the transcript identifiers correspond to transcript isoform sets, and each of the transcript isoform sets is encoded by a gene; (ii) transcript identifiers to corresponding transcript expression values in diseased tissue samples, and (iii) transcript identifiers to corresponding transcript expression values in non-diseased tissue samples; and instructions executable by the one or more microprocessors configured to perform the following method (a) receiving user input comprising (i) a defined disease selected from a plurality of defined diseases, where each of the defined diseases corresponds to diseased tissue samples, (ii) a defined minimum transcript expression value threshold for diseased tissue samples, (iii) a defined maximum or a defined median transcript expression value threshold for non-diseased tissues, where each of the non-diseased tissues corresponds to non-diseased tissue samples, and (iv) a defined minimum sample sub-population percentage; (b) identifying in the database one or more transcript identifiers meeting the following criteria (1) and (2): (1) a corresponding transcript expression value in diseased tissue samples corresponding to the input defined disease of (a)(i) is greater than the input defined minimum transcript expression value threshold of (a)(ii), and (2) a corresponding transcript expression value in non-diseased tissue is less than the input defined maximum or defined median transcript expression value threshold of (a)(iii), for a percentage of diseased samples corresponding to the input defined disease of (a)(i) greater than the defined minimum sub-population percentage of (a)(iv); and (c) outputting a list comprising a transcript identifier for each of one or more transcripts identified in (b).

Also provided in certain aspects are systems, machines, or apparatuses comprising one or more microprocessors and memory, the memory comprising a database comprising one or more tables that relate (i) transcript identifiers to genes, where at least a portion of the transcript identifiers correspond to transcript isoform sets, and each of the transcript isoform sets is encoded by a gene; (ii) transcript identifiers to corresponding transcript expression values in diseased tissue samples, and (iii) transcript identifiers to corresponding transcript expression values in non-diseased tissue samples; and instructions executable by the one or more microprocessors configured to perform the following method (a) receiving user input comprising (i) a defined disease selected from a plurality of defined diseases, where each of the defined diseases corresponds to diseased tissue samples, (ii) a defined transcript expression ratio threshold, where the transcript expression ratio is a ratio of a transcript expression value for diseased tissue samples to a transcript expression value for non-diseased tissues, where each of the non-diseased tissues corresponds to non-diseased tissue samples, and (iii) a defined minimum sample sub-population percentage; (b) identifying in the database one or more transcript identifiers having a corresponding transcript expression ratio for the input defined disease of (a)(i) that is greater than the input defined transcript expression ratio threshold of (a)(ii), for a percentage of diseased samples corresponding to the input defined disease of (a)(i) greater than the defined minimum sub-population percentage of (a)(iii); and (c) outputting a list comprising a transcript identifier for each of one or more transcripts identified in (b).

Also provided in certain aspects are computer-implemented methods for analyzing a polypeptide comprising (a) identifying one or more transcript identifiers in a database comprising one or more tables that relate (i) transcript identifiers to genes, where at least a portion of the transcript identifiers correspond to transcript isoform sets, and each of the transcript isoform sets is encoded by a gene; (ii) transcript identifiers to corresponding transcript expression values in diseased tissue samples, and (iii) transcript identifiers to corresponding transcript expression values in non-diseased tissue samples; (b) receiving user selection of a transcript identifier; and (c) outputting one or more of (i) a three-dimensional structure corresponding to a polypeptide linear sequence corresponding to the selected transcript identifier, and (ii) functional polypeptide domain information for a polypeptide linear sequence corresponding to the selected transcript identifier, where the one or more tables of the database relate three-dimensional structure coordinates and functional polypeptide domain information to the polypeptide linear sequence.

Also provided in certain aspects are methods for generating a database comprising (i) relating transcript identifiers to genes, where at least a portion of the transcript identifiers correspond to transcript isoform sets, and each of the transcript isoform sets is encoded by a gene; (ii) relating transcript identifiers to corresponding transcript expression values in diseased tissue samples for a plurality of defined diseases, and (iii) relating percentages of diseased tissue samples for each defined disease to corresponding transcript identifiers, where the percentages are based on an amount of diseased tissue samples corresponding to a defined disease for which transcript expression values of a corresponding transcript identifier exceed a defined minimum transcript expression value threshold, or (iii′) relating percentages of diseased tissue samples for each defined disease to corresponding transcript identifiers, where the percentages are based on an amount of diseased tissue samples corresponding to a defined disease for which transcript expression ratios of a corresponding transcript identifier exceed a defined transcript expression ratio threshold.

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 shows an example database construction pipeline.

FIGS. 2A and 2B show an example interface for user input (e.g., tumor search query submission).

FIGS. 3A-3C show an example interface for results. FIG. 3A (top panel) shows a list of genes/isoforms that meet search criterion—one is selected, and displays in the middle and lower panels are populated with information about the selected isoform. In this example, the highlighted transcript has strong tumor expression in 67% of patients. FIG. 3A (middle panel) shows expression of other transcripts for the selected gene including max norm tissue. FIG. 3A (bottom panel) shows a multi sequence alignment, indicating canonical transcript (top), selected transcript (highlight), and unique peptide target (arrow). FIG. 3B shows a box plot of expression in 47 GTEx normal tissues (very low). Only outliers (black circles) showed expression, and some expression was observed in whole blood with max of ˜0 TPM. FIG. 3C shows a box plot of isoform expression in tumor showing strong expression in Thymoma.

FIG. 4 shows an example protein analysis tool launch for a selected transcript.

FIG. 5 shows an example gene summary output in a protein analysis tool for a selected transcript.

FIG. 6 shows an example sequence and structure output in a protein analysis tool for a selected transcript.

FIG. 7 shows an example images output in a protein analysis tool for a selected transcript. In this example, searches are done using Pub Med Central and Bing (or Google) to locate relevant information about the gene of interest. Searches may include additional search terms that are tuned over time. Results include linkouts to the source information.

FIG. 8 shows an example publications output in a protein analysis tool for a selected transcript. In this example, publication searches are done in Pub Med using several search terms that include the gene of interest. Results include linkouts to the articles.

FIG. 9 shows an example of a subpopulation of subjects with high TPM Log 2 values for a particular tumor type.

FIG. 10 shows an example of high TPM Log 2 values for at least one subject per tumor type across multiple tumor types.

FIG. 11 shows an example sequence and structure output in a protein analysis tool for a selected transcript. The output page includes a sequence panel, a domain panel, and a structure panel.

FIG. 12 shows a functional aspect within the structure panel in a protein analysis tool for a selected transcript.

FIG. 13 shows domain highlighting functionality for an example sequence and structure output in a protein analysis tool for a selected transcript.

FIG. 14 shows isoform fragment highlighting functionality for an example sequence and structure output in a protein analysis tool for a selected transcript.

FIG. 15 shows isoform position highlighting functionality for an example sequence and structure output in a protein analysis tool for a selected transcript.

FIG. 16 and FIG. 17 show color coding functionality for an example sequence and structure output in a protein analysis tool for a selected transcript.

FIG. 18 shows differential isoform fragment highlighting functionality for an example sequence and structure output in a protein analysis tool for a selected transcript.

FIG. 19 shows 3D structure differential isoform fragment highlighting functionality for an example sequence and structure output in a protein analysis tool for a selected transcript.

FIG. 20 shows a mechanism for selecting a 3D protein structure for an example sequence and structure output in a protein analysis tool for a selected transcript.

FIG. 21 shows a solvent-accessible surface area display option for a 3D protein structure for a structure panel in a protein analysis tool for a selected transcript.

FIG. 22 shows an isoform mapped display option for a 3D protein structure for a structure panel in a protein analysis tool for a selected transcript.

FIG. 23 shows an example interface for user input (e.g., gene search query submission).

FIG. 24 illustrates an implementation of a system as described herein.

FIGS. 25A-25C show an example interface for user input (e.g., tumor search query submission).

FIG. 26 shows a box plot of isoform expression in 47 GTEx normal tissues showing high expression in testis.

FIG. 27 shows a box plot of isoform expression in tumor samples showing strong expression in certain tumors (i.e., esophageal carcinoma, ovarian serous cystadenocarcinoma, and stomach adenocarcinoma (circled)).

FIG. 28 (top panel) shows a list of genes/isoforms that meet search criterion—one is selected, and displays in the middle and lower panels are populated with information about the selected isoform.

FIG. 28 (middle panel) shows a multi sequence alignment, indicating canonical transcript (second sequence from the top), selected transcript (highlight), and partially unique peptide target (arrow).

FIG. 28 (bottom panel) shows expression of other transcripts for the selected gene.

FIG. 29 shows a box plot of combined MSLN partially unique transcript expression in tumor samples.

### DETAILED DESCRIPTION

Provided herein are methods and systems for identifying disease-associated transcript isoforms. Also provided herein are methods and systems for analyzing peptides and polypeptides encoded by the disease-associated transcript isoforms. In some aspects, identifying disease-associated transcript isoforms involves a comparison of transcripts from diseased samples to transcripts from a variety of tissues from normal samples. In some aspects, a method herein involves identifying a subpopulation of diseased samples having elevated expression of a disease-associated transcript isoform. In some aspects, a method herein involves identifying a disease-associated transcript isoform having elevated expression across multiple disease types.

### Differential Expression

A particular gene can give rise to different polypeptide or peptide variants according to one or more alterations at the DNA level (e.g., point mutation event) or mRNA level (e.g., alternative splice event). A particular gene can encode two or more polypeptide variants or peptide variants that are distinguished, for example, by at least one single amino acid substitution, at least one single amino acid insertion, at least one single amino acid deletion, at least one substitution of two or more consecutive amino acids, at least one insertion of two or more consecutive amino acids, at least one deletion of two or more consecutive amino acids, or a combination thereof.

Methods described herein may include an expression analysis of a variant subsequence (e.g., a splice variant subsequence). In some embodiments, a method described herein includes an analysis of differential expression (e.g., expression of a gene, variant (e.g., splice variant), or variant subsequence (e.g. splice variant subsequence) in different organs or tissues, expression of a gene, variant (e.g., splice variant), or variant subsequence (e.g. splice variant subsequence) in different subjects, expression of a gene, variant (e.g., splice variant), or variant subsequence (e.g. splice variant subsequence) in healthy vs. disease organs or tissues, expression of a gene, variant (e.g., splice variant), or variant subsequence (e.g. splice variant subsequence) in healthy vs. disease subjects). In some embodiments, a differential expression analysis includes comparing gene, variant (e.g., splice variant), or variant subsequence (e.g. splice variant subsequence) expression in tumors vs. surrounding tissue (e.g., in the same subject). In some embodiments, a differential expression analysis includes comparing gene, variant (e.g., splice variant), or variant subsequence (e.g. splice variant subsequence) expression in tumors (e.g., from one or more disease subjects) vs. corresponding tissue (e.g., from one or more healthy subjects).

Corresponding tissue generally refers to an equivalent organ or tissue in a healthy subject that is cancerous in a disease subject. For example, if a disease subject has prostate cancer, corresponding tissue would refer to prostate tissue from a healthy subject. In some embodiments, a differential expression analysis includes comparing gene, variant (e.g., splice variant), or variant subsequence (e.g. splice variant subsequence) expression in tumors (e.g., from one or more disease subjects) vs. normal tissue (e.g., one or more tissues from one or more healthy subjects).

Any suitable method for determining or measuring levels of gene, variant (e.g., splice variant), or variant subsequence (e.g. splice variant subsequence) expression may be used in a gene, variant (e.g., splice variant), or variant subsequence (e.g. splice variant subsequence) expression analysis and/or a differential expression analysis. Examples of methods for measuring expression levels include qPCR, RT-qPCR, RNA-Seq, microarray, northern blot, differential display, and RNase protection assay.

In some embodiments, expression levels may be measured using a quantifiable amplification method. For example, expression levels may be measured using a quantitative PCR (qPCR) approach (e.g., on cDNA generated from mRNA from a sample), or a reverse transcriptase quantitative PCR (RT-qPCR) approach (e.g., on mRNA from a sample). Quantitative PCR (qPCR), which also may be referred to a real-time PCR, monitors the amplification of a targeted nucleic acid molecule during a PCR reaction (i.e., in real time). This method may be used quantitatively (quantitative real-time PCR) and semi-quantitatively (i.e., above/below a certain amount of nucleic acid molecules; semi-quantitative real-time PCR. Methods for qPCR include use of non-specific fluorescent dyes that intercalate with double-stranded DNA, and sequence-specific DNA probes labelled with a fluorescent reporter, which generally allows detection after hybridization of the probe with its complementary sequence. Quantitative PCR methods typically are performed in a thermal cycler with the capacity to illuminate each sample with a beam of light of at least one specified wavelength and detect the fluorescence emitted by an excited fluorophore.

For non-specific detection, a DNA-binding dye binds to all double-stranded (ds) DNA during PCR. An increase in DNA product during PCR therefore leads to an increase in fluorescence intensity measured at each cycle. For qPCR using dsDNA dyes, the reaction typically is prepared like a basic PCR reaction, with the addition of fluorescent dsDNA dye. Then the reaction is run in a real-time PCR instrument, and after each cycle, the intensity of fluorescence is measured with a detector (the dye only fluoresces when bound to the dsDNA (i.e., the PCR product)). In certain applications, multiple target sequences may be monitored in a tube by using different types of dyes. For specific detection, fluorescent reporter probes detect only the DNA containing the sequence complementary to the probe. Accordingly, use of the reporter probe increases specificity, and enables performing the technique even in the presence of other dsDNA. Using different types of labels, fluorescent probes may be used in multiplex assays for monitoring several target sequences in the same tube. This method typically uses a DNA-based probe with a fluorescent reporter at one end and a quencher of fluorescence at the opposite end of the probe. The close proximity of the reporter to the quencher prevents detection of its fluorescence. During PCR, the probe is broken down by the 5′ to 3′ exonuclease activity of the polymerase, which breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter.

In some embodiments, expression levels may be measured using a sequencing process (e.g., RNA sequencing (RNA-Seq)). RNA-Seq typically uses high-throughput sequencing to detect the presence and/or measure the quantity of RNA in a sample. In certain applications, RNA-Seq allows for detection and/or measurement of alternative spliced transcripts, post-transcriptional modifications, gene fusion, mutations/SNPs, changes in gene expression over time, and/or differential expression. RNA-Seq can analyze different populations of RNA, which may include mRNA transcripts, total RNA, small RNA (e.g., miRNA), tRNA, and ribosomal RNA. RNA-Seq also may be used to assess exon/intron boundaries.

A disease-associated isoform identification system described herein can identify a disease-associated polypeptide or peptide variant of a particular gene. Stated another way, a disease-associated isoform identification system can identify a disease-associated variant (e.g., splice variant) of a particular gene, and an amino acid sequence of one or more variants of the gene may be aligned and/or outputted by a disease-associated isoform identification system. In certain instances expression level values are within a dataset described herein. In certain instances a dataset is received and/or stored in memory, and sometimes a dataset is in a database. A dataset may contain expression level values for transcripts in disease samples and non-disease samples from multiple tissues. Transcripts in a dataset can be virtual RNA transcripts (e.g., mRNA transcripts) and representative polynucleotide sequences (e.g., RNA, DNA and/or cDNA sequences) of transcripts or portions thereof can be included in a dataset. Transcripts in a dataset can correspond to amino acid sequence variants encoded by a gene, and a database can include representative amino acid sequences and/or amino acid subsequence corresponding to (e.g., translated from) transcripts or portions thereof. Transcripts can correspond to variants (e.g., splice variants) encoded by one or more genes, and a database can include expression level values associated with polynucleotide sequences, amino acid sequences and/or amino acid subsequence corresponding to transcripts or portions thereof.

A disease-associated isoform identification system described herein can include or can receive a dataset containing amino acid sequences of polypeptides and peptides encoded by genes and associated expression level information. If there are three polypeptide variants for a particular gene, for example, the system can include or receive (i) an amino acid sequence for each variant, and (ii) associated expression level information for each variant. Amino acid sequence information and associated expression level information can be stored in a system in any suitable format (e.g., a .tar archive). Expression level information stored in a system can exist in a system in any suitable manner, and sometimes exists as normalized expression level information.

Candidate disease-associated polypeptides or peptide variants (e.g., tumor-specific isoforms) may be defined by comparing the isoform expression levels between normal and disease (e.g., tumor) conditions. In one embodiment, candidate tumor-specific isoforms are defined by comparing the isoform expression levels between normal and tumor conditions, by calculating the median TPM-based fold change, and the presence or absence of one candidate tumor-specific isoform. In another embodiment, candidate tumor-specific isoforms are defined by comparing isoform expression levels between normal and tumor conditions, by calculating the median FPKM-based fold change, and the presence or absence of one candidate tumor-specific isoform. In another embodiment, candidate tumor-specific isoforms are defined by comparing isoform expression levels between normal and tumor conditions, by calculating the median RPKM-based fold change, and the presence or absence of one candidate tumor-specific isoform. In another embodiment, candidate tumor-specific isoforms are defined by comparing isoform expression levels between normal and tumor conditions, by calculating the median RSEM-based fold change, and the presence or absence of one candidate tumor-specific isoform.

A dataset of a disease-associated isoform identification system, or a dataset received by a disease-associated isoform identification system, can include (i) expression level information associated with amino acid sequence variants for disease samples, and (ii) expression level information associated with amino acid sequence variants for non-disease samples. A dataset can include expression levels (e.g., average expression levels) of transcripts associated with particular amino acid sequence variants for disease samples. A dataset can include expression levels (e.g., average expression levels) of transcripts associated with particular amino acid sequence variants for non-disease samples. A dataset can include a composite expression level associated with a particular amino acid sequence variant for non-disease samples (e.g., for all non-disease samples or a subset of non-disease samples). A composite expression level often is an average of the average expression level in a dataset for each tissue of origin of non-disease samples (e.g., all non-diseased samples in a dataset or a subset of non-disease samples in a dataset).

Each disease sample in a dataset of a disease-associated isoform identification system can be associated with a tissue of origin. In certain configurations, each disease sample in a dataset of a disease-associated isoform identification system can be associated with (i) a tissue of origin, and (ii) matched expression level values for each transcript derived from non-disease tissue adjacent to the disease tissue. In certain configurations, each disease sample in a dataset of a disease-associated isoform identification system can be associated with a tissue of origin, and is not associated with matched expression level values for each transcript derived from non-disease tissue adjacent to the disease tissue. Sample information can be from any suitable dataset or combined dataset, a non-limiting example of which includes a datasets from TCGA (World Wide Web Uniform Resource Locator cancer.gov/about-nci/organization/ccg/research/structural-genomics/tcga), TARGET (World Wide Web Uniform Resource Locator ocg.cancer.gov/programs/target), and GTEx (World Wide Web Uniform Resource Locator gtexportal.org/home/).

A disease sample generally is from a portion of a tissue of an organism identified as being diseased, and a non-disease sample generally is from a portion of a tissue of an organism identified as not being diseased. A disease sample and a non-disease sample sometimes are from the same subject, and sometimes are not from the same subject. A disease sample sometimes is from a portion of a tissue of an organism identified as being diseased, and a non-disease sample sometimes is from a portion of an adjacent tissue of an organism identified as not being diseased. For example, a disease sample sometimes is from a cancer tumor and a non-disease sample sometimes is from a non-tumor tissue adjacent to the tumor in the same subject. In some configurations, a disease-associated isoform identification system herein excludes datasets from non-tumor tissue adjacent to tumor tissue, and instead includes datasets from non-disease samples from subjects identified as not having a disease (e.g., datasets from GTEx). In some instances, the datasets from non-disease samples include expression data from multiple non-disease tissues.

A disease sometimes is a condition, and a disease or condition sometimes is diagnosed, inferred or suspected for a subject. Non-limiting examples of disease samples include samples from subjects having or suspected of having Alzheimer's disease, Parkinson's disease, Lupus, IPEX syndrome, diabetes, rheumatoid arthritis, influenza, pneumonia, or tuberculosis. A disease sample can be a cancer sample, and non-limiting examples of cancer samples include samples from subjects having or suspected of having a cancer type described herein.

A disease-associated isoform identification system can compare, for a selected disease: (i) an expression level (e.g., average expression level) of a particular amino acid sequence variant for disease samples, to (ii) a composite expression level of the particular amino acid sequence variant for non-disease samples (e.g., for all non-disease samples or a subset of non-disease samples). A disease-associated isoform identification system can compare, for a selected gene: (i) an expression level (e.g., average expression level) of a particular amino acid sequence variant for disease samples, to (ii) a composite expression level of the particular amino acid sequence variant for non-disease samples (e.g., for all non-disease samples or a subset of non-disease samples).

A disease-associated isoform identification system can perform several computations using expression level information in a dataset. A system can, for example: (a) compute an average expression level value for each transcript for disease samples; and/or (b) compute for each amino acid sequence variant a “related variant” value for disease samples and a “related variant” value for non-disease samples, where the “related variant” value is (i) the average (e.g., mean or median) expression level for the variant, divided by (ii) the sum of average expression level values for each variant of the gene; and/or (c) compute for each amino acid sequence variant a “fold change” value, where the “fold change” value is (i) the average expression level for the amino acid sequence variant in disease samples, divided by (ii) the average expression level for the amino acid sequence variant in non-disease samples. The “related variant” value can be expressed as a percentage referred to as an “expression percentage.” A computation described for part (a) also can include matching each average expression level value for each transcript with (i) a composite average expression level for the transcript for all non-disease samples or a subset of non-disease samples, and/or (ii) a highest tissue expression level identified from all non-disease samples or a subset of non-disease samples for the transcript. While each of the related variant value and the fold change value described in part (b) and part (c) is computed by dividing (i) by (ii), each ratio independently may be computed by dividing (ii) by (i).

After performing computations (a) and/or (b) and/or (c) described in the preceding paragraph, a disease-associated isoform identification system can generate a “disease sample only” variant list. Each variant selected for the list is expressed in disease samples but generally not expressed in non-diseased samples, where “not expressed” is defined as an expression level (e.g., TPM expression level) of less than 0.00001. The resulting list often is sorted by expression level value such that the most highly-expressed “disease sample only” variants are at the top of the list. A configurable cutoff value can be applied to expression level in disease samples, whereby only amino acid variants are displayed that are associated with a value greater than or equal to the value associated with the cutoff. For example, a threshold of greater-than-or-equal-to a TPM expression level of 1.0, and an expression percentage of greater than 10% can be applied.

After performing computations (a) and/or (b) and/or (c) described above, a disease-associated isoform identification system can generate a “disease sample specific” variant list. Each variant selected for the list can be (i) the dominant variant in disease samples, and/or (ii) not the dominant variant in non-disease samples.

After performing computations (a) and/or (b) and/or (c) described above, a disease-associated isoform identification system can generate a “disease upregulated” variant list. Each variant selected for the list exhibits a fold change value equal-to-or-greater-than a threshold value. The fold change expression level threshold can be configurable by a user, and a threshold value can be a two-fold threshold value, for example.

A disease-associated isoform identification system can generate a multi-sequence alignment (MSA) for each variant included in a list, which facilitates identification of an amino acid subsequence present in a particular variant that is not present in at least one other variant of a particular gene. A MSA often aligns a variant amino acid sequence with an amino acid sequence of at least one other variant encoded by the same gene. A MSA can be generated using any suitable sequence alignment algorithm, non-limiting examples of which include Clustal (e.g., ClustalW, ClustalW2, Clustal Omega), Multiple Alignment using Fast Fourier Transform (MAFFT), T-COFFEE, M-COFFEE, LALIGN, PSAlign, PRRN, PRRP, DIALIGN, MUSCLE, MergeAlign, Partial-Order Alignment (POA), Sequence Alignment and Modeling System (SAM), HMMER, PRANK, PAGAN, ProGraphMSA, MEME, MAST and EDNA. A disease-associated isoform identification system can generate a MSA based on a gene identifier, which can involve synching amino acid sequence databases having disparate gene identifier information. A non-limiting example of such a synching process is described hereafter. A TCGA variant model was based on the hg19 2009 version of the UCSC gene dataset (gene models built by UCSC as part of a genome browser). The UCSC table known as GenePep was downloaded for hg19 to obtain the protein sequence of each of the variants used in the TCGA analysis. Multiple versions of the hg19 USC gene models were released over time; the UCSC gene hg19 version 12 from 2009 was obtained that matched the gene models used in the TCGA analysis. A gene symbol scheme was constructed for transcript mapping from the TCGA reference dataset and the GenePep table was used to build a Sqlite database that supports looking up a gene symbol and returning all the protein sequences of the transcripts of the gene. These were then run through a multi-sequence alignment program and then through a format routine. The resulting structure allowed for a protein alignment program to receive a gene symbol and produces a multi-sequence alignment that can be used to identify sections specific to a tumor-associated variant.

A disease-associated isoform identification system can generate a box plot of expression level values for disease samples and/or non-disease samples by tissue for a given variant. Data from any suitable dataset can be utilized for such a box plot, non-limiting examples of which include the TCGA dataset described herein, the TARGET dataset described herein, and the GTEx dataset described herein. A disease-associated isoform identification system can generate a box plot of expression level values of a variant in disease and/or non-disease samples for different tissues, and can generate: (i) upper whisker, lower whisker, upper quartile, lower quartile, and/or an average of the distribution of values for a selected disease type; and/or (ii) a maximum expression level value (e.g., maximum TPM value) for a non-disease sample from a relevant tissue. In some configurations, a disease-associated isoform identification system can generate a box plot of expression level values of a variant in disease and/or non-disease samples for different tissues, and can generate one or more of: average transcript expression value, first quantile of average transcript expression value, third quantile of average transcript expression value, maximum transcript expression value whisker, minimum transcript expression value whisker, and outlier designations for transcripts.

A disease-associated isoform identification system can include an interface (e.g., a graphic interface) that facilitates selection of one or more features described herein. An interface can facilitate user interaction with a computing device by way of graphical elements, text-based elements, check boxes, drop-down menus, and the like. Graphical elements may include, for example, windows, buttons, icons, widgets, sliders, text boxes, spinner controls, and menus (e.g., drop down menus) that can be selected, highlighted, moved, and the like. Graphical elements may be manipulated using a touch screen, keyboard, stylus, and/or pointing device (e.g., mouse). In certain configurations, two or more screens of a user interface are linked together into a workflow, and navigation between screens may be possible in some instances. User interfaces may be web-based, for example, with parts of each page being loaded from a server and/or a database, or natively-compiled to execute on a user device with or without network availability.

A disease-associated isoform identification system can include an interface (e.g., a graphic interface) that facilitates selection of (i) a disease of interest (e.g., a tumor type of interest), (ii) a gene of interest according to a gene identifier (e.g., gene name, a gene tag (i.e., an abbreviated version of a gene name), accession number), or (iii) a tissue type of interest (e.g., TCGA, TARGET, and/or GTEx tissue type of interest). An interface can facilitate selection of one or more filters. An interface can facilitate selection of one or more filters that enhance identification of variants that are immunologic and/or can be immunologically targeted. An interface can facilitate selection of an expression level threshold filter that permits listing of only transcripts and/or variants associated with a minimum disease sample expression level (e.g., minimum disease sample TPM; minimum tumor TPM). An interface can facilitate input of an expression level threshold filter that permits listing only of transcripts and/or variants associated with a maximum non-disease sample expression level (e.g., maximum non-disease sample TPM; maximum normal TPM) or a median non-disease sample expression level (e.g., median non-disease sample TPM; median normal TPM). An interface can facilitate input of filter that permits listing only of transcripts and/or variants associated with a selected subpopulation (e.g., a minimum percentage of samples meeting an expression level minimum (e.g., for diseased samples) and/or expression level maximum or median (e.g., for non-diseased samples)). In certain configurations, an interface can facilitate selection of an expression ratio threshold filter that permits listing of only transcripts and/or variants associated with a minimum ratio value. An interface can facilitate selection of a filter that permits listing of only transcripts and/or variants that encode a cell-surface polypeptide (e.g., based on an annotation in a dataset specifying that a particular gene encodes a cell-surface polypeptide). An interface can facilitate selection of a filter that permits listing of only transcripts and/or variants having at least one insertion of a single amino acid or two or more consecutive amino acids relative to a canonical amino acid sequence (e.g., a unique peptide).

An interface of a disease-associated isoform identification system can display a list of variants. An interface can output a sorted list of variants with the strongest disease/non-disease differences at the top of the list. In some configurations, two types of lists are available for each tissue: (i) variants expressed in disease samples but not expressed in non-disease samples, and (ii) variants expressed at significantly higher levels in disease samples than non-disease samples. An interface also can allow selection of a particular variant in a list for display of additional output pertaining to the variant, as described in further detail herein. For example, additional output can include (i) a detail panel showing a multi-sequence alignment for the selected variant and all other variants encoded by the gene; (ii) a box plot of expression of the variant in disease and/or non-disease samples for different tissues (e.g., a box plot for TCGA dataset and/or a box plot for GTEx dataset); (iii) three dimensional protein structure information for a selected variant; and/or (iv) protein domain information for a selected variant.

In certain instances, a gene identifier or transcript identifier (e.g., gene name, a gene tag (i.e., an abbreviated version of a gene name), transcript name, a transcript tag (i.e., an abbreviated version of a transcript name), accession number) can be inputted into a disease-associated isoform identification system, and an algorithm associated with the system can identify the gene identifier and/or transcript identifier in a pre-compiled database and retrieve the associated amino acid sequence from the database. An amino acid sequence can be retrieved by an algorithm associated with a disease-associated isoform identification system from a NCBI Protein database (e.g., World Wide Web Uniform Resource Locator ncbi.nlm.nih.gov/protein), for example. In certain instances, an amino acid sequence can be directly inputted into the disease-associated isoform identification system (e.g., copying and pasting an amino acid sequence). An input amino acid sequence can be a polypeptide amino acid sequence or a peptide amino acid sequence (e.g., a polypeptide or a peptide encoded by a gene or by a mRNA), or a portion of a polypeptide amino acid sequence or a peptide amino acid sequence, for example.

### Disease-Associated Isoform Identification

Provided herein are methods and systems for identifying a particular transcript of a gene. A system herein may refer to a machine or apparatus, in certain embodiments. A particular transcript of a gene may be referred to an isoform. A transcript or an isoform may refer to a transcript variant or an isoform variant. A transcript variant or an isoform variant may refer to a splice variant. In some embodiments, a transcript is a disease-associated transcript. In some embodiments, a transcript is expressed at a level in diseased tissue higher than a level the transcript is expressed in non-diseased tissue. In some embodiments, a transcript is a cancer-associated transcript. In some embodiments, a transcript is expressed at a level in cancerous tissue higher than a level the transcript is expressed in non-cancer tissue. In some embodiments, a transcript is a tumor-associated transcript. In some embodiments, a transcript is expressed at a level in tumor tissue higher than a level the transcript is expressed in non-tumor tissue. In some embodiments, a transcript is expressed at a level in diseased, cancerous, and/or tumor tissues at least about 5% higher than a level the transcript is expressed in non-diseased, non-cancer, and/or non-tumor tissues. For example, a transcript may be expressed at a level in diseased, cancerous, and/or tumor tissues at least about 10%, 20%, 30%, 40%, 50%, 60%, 70%, 80%, 90%, 100%, 200%, or 500% higher than a level the transcript is expressed in non-diseased, non-cancer, and/or non-tumor tissues. In some embodiments, a transcript is expressed at a level in diseased, cancerous, and/or tumor tissues at least about 1.5 times higher than a level the transcript is expressed in non-diseased, non-cancer, and/or non-tumor tissues. For example, a transcript may be expressed at a level in diseased, cancerous, and/or tumor tissues at least about 2 times, 3 times, 4 times, 5 times, 6 times, 7 times, 8 times, 9 times, 10 times, 20 times, 30 times, 40 times, 50 times, 60 times, 70 times, 80 times, 90 times, or 100 times higher than a level the transcript is expressed in non-diseased, non-cancer, and/or non-tumor tissues. The term disease-associated transcript may be used interchangeably herein with the terms cancer-associated transcript and/or tumor-associated transcript. The term diseased tissue may be used interchangeably herein with the terms cancerous tissue, cancerous tissue sample, tumor tissue and/or tumor tissue sample. The term non-diseased tissue may be used interchangeably herein with the terms non-cancer tissue, non-cancer tissue sample, non-tumor tissue, and/or non-tumor tissue sample.

In some embodiments, a transcript is expressed at a level in a defined subpopulation of diseased tissue samples higher than a level the transcript is expressed in non-diseased tissue samples. A defined subpopulation of diseased samples generally refers to a selected portion of total diseased samples for which expression data has been collected. A selected portion of diseased samples may be expressed as a fraction or percentage of the total number of diseased samples for which expression data has been collected. A defined subpopulation of diseased samples may refer to a defined subpopulation of samples for a particular disease. A defined subpopulation of diseased samples may refer a defined subpopulation for multiple diseases.

Database

In some embodiments, a method herein includes use of a database. In some embodiments, a system herein includes a database. A database may comprise one or more tables that relate two or more features described herein. Two or more features may be related in any suitable tabular format (e.g., rows and columns, spreadsheet, and the like). In some embodiments, a database comprises one or more tables that relate transcript identifiers (e.g., transcript name, a transcript tag (i.e., an abbreviated version of a transcript name), accession number) to one or more features described herein. For example, one or more tables in a database may relate one or more of: transcript identifiers to genes; transcript identifiers to corresponding transcript expression values in diseased tissue samples; transcript identifiers to corresponding transcript expression values in non-diseased tissue samples; percentages of diseased tissue samples to corresponding transcript identifiers (e.g., for one or more defined diseases); average transcript expression values to corresponding transcript identifiers (e.g., for one or more defined diseases); average transcript expression values in diseased tissue samples, average transcript expression values for all non-diseased samples or a subset of non-disease samples, maximum transcript expression values of non-diseased samples, and non-diseased tissues corresponding to maximum transcript expression values, to corresponding transcript identifiers; average transcript expression values in diseased tissue samples, average transcript expression values for all non-diseased samples or a subset of non-disease samples, median transcript expression values of non-diseased samples, and non-diseased tissues corresponding to median transcript expression values, to corresponding transcript identifiers; transcript expression values and box plot statistics with corresponding transcript identifiers; and polypeptide linear sequences to corresponding transcript identifiers. In some embodiments, a database comprises one or more tables that relate three-dimensional structure coordinates and functional polypeptide domain information to the polypeptide linear sequences.

In some embodiments, a database described herein comprises one or more of the following tables: a samples table comprising one record for each sample and including phenotype information for each sample; a tissue table comprising one row for each tissue type and comprising tissues corresponding to diseased tissue samples and tissues corresponding to non-diseased tissue samples; a transcript table relating gene identifiers to corresponding transcript identifiers; a non-diseased sample statistics table relating transcript expression values to corresponding transcript identifiers for non-diseased samples, and comprising one or more of: average transcript expression value, first quantile of average transcript expression value, third quantile of average transcript expression value, maximum transcript expression value whisker, minimum transcript expression value whisker, and outlier designations for transcripts of non-diseased samples, for corresponding transcript identifiers; a non-diseased sample statistics by tissue table relating transcript expression values to corresponding transcript identifiers categorized by tissue, and comprising one or more of: average transcript expression value, first quantile of average transcript expression value, third quantile of average transcript expression value, maximum transcript expression value whisker, minimum transcript expression value whisker, outlier designations for transcripts of samples in each tissue, and the tissue having the highest expression value for each transcript identifier, for corresponding transcript identifiers; a diseased sample statistics by tissue table relating transcript expression values to transcript identifiers categorized by tissue, and comprising one or more of: average transcript expression value, first quantile of average transcript expression value, third quantile of average transcript expression value, maximum transcript expression value whisker, minimum transcript expression value whisker, outlier designations for transcripts of samples in each tissue; a diseased sample percentage table relating percentages of diseased tissue samples for each defined disease to corresponding transcript identifiers; an aligned linear sequences table relating transcript identifiers to corresponding linear polypeptide sequences; a transcript table, where the transcript table identifies a subset of gene identifiers each corresponding to a gene encoding a cell surface protein; a transcript table, where the transcript table identifies a subset of transcript identifiers each corresponding to a transcript encoding a unique polypeptide comprising an insertion, deletion or substitution of one or more amino acids relative to polypeptides encoded by other transcript isoforms of the same gene; and a transcript table, where the transcript table identifies a subset of transcript identifiers each as a primary transcript having an expression value (e.g., a general expression value) higher than the expression values corresponding to other transcript isoforms encoded by the same gene.

In some embodiments, for a diseased sample percentage table, each percentage is calculated based on transcript expression values for diseased tissue samples for which transcript expression values exceed a defined minimum transcript expression value threshold, and for each defined minimum sample sub-population percentage. In some embodiments, a diseased sample statistics by tissue table relates average transcript expression values for each defined disease to corresponding transcript identifiers. Each average transcript expression value may be calculated based on transcript expression values for diseased tissue samples for which transcript expression values exceed a defined minimum transcript expression value threshold, and for each defined minimum transcript expression value threshold of a plurality of defined minimum transcript expression value thresholds. Average transcript expression values may be mean transcript expression values. Average transcript expression values may be median transcript expression values.

In some embodiments, a database relates transcript identifiers to genes. In some embodiments, at least a portion of the transcript identifiers correspond to transcript isoform sets. Generally, each transcript isoform set is encoded by a gene (i.e., one gene per isoform set). Isoform sets may include one or more of a canonical sequence, a consensus sequence, and a variant sequence for a selected gene. In some embodiments, an isoform set includes a plurality of variant sequences for a selected gene. Variant sequences in an isoform set may include splice variants, for example.

In some embodiments, a database relates transcript identifiers to corresponding transcript expression values in diseased tissue samples. In some embodiments, a database relates transcript identifiers to corresponding transcript expression values in non-diseased tissue samples. Transcript expression value information (e.g., for diseased tissue samples and/or non-diseased tissue sample) can exist, for example, as transcripts per million (TPM) values, fragments per kilobase per million reads mapped (FPKM) values, reads per kilobase per million reads mapped (RPKM) values, RNA-seq by expectation-maximization (RSEM) values, or combination of such values. In some embodiments, transcript expression values are transcripts per million (TPM) values. TPM values generally are normalized expression level values, and a TPM value for a particular gene/transcript represents the number of RNA molecules in a sample for every one million RNA molecules in the sample. A TPM value generally is determined for RNA-seq samples. In some embodiments, TPM values are or have been generated by a RNA-Seq by Expectation-Maximization (RSEM) algorithm (Li and Dewey (2011) BMC Bioinformatics 12:323 pages 1-16; incorporated by reference herein). TPM values may be expressed as logarithmic TPM values (e.g., binary logarithmic values). For example, TPM values may include Log 2 TPM values. Minimum Log 2 TPM values and/or TPM value thresholds (e.g., for diseased tissue samples) may include various precomputed tumor expression thresholds (e.g., to improve processing speed, make the database manageable, and make performance of the search tractable). Minimum Log 2 TPM values and/or TPM value thresholds (e.g., for diseased tissue samples) may include various precomputed tumor expression thresholds ranging from 1 to 10. Minimum Log 2 TPM values and/or TPM value thresholds (e.g., for diseased tissue samples) may include various precomputed tumor expression thresholds ranging from 1 to 10, incremented by 1. Non-limiting examples of minimum Log 2 TPM values and/or TPM value thresholds (e.g., for diseased tissue samples) include 1, 2, 3, 4, 5, 6, 7, 8, 9, and 10. In some embodiments, a minimum Log 2 TPM value and/or TPM value threshold (e.g., for diseased tissue samples) is 2, 3, or 4. Maximum Log 2 TPM values and/or TPM value thresholds (e.g., for non-diseased tissues) can be any number and generally range from about −9.9 (for very low expression) to 18 (for extremely high expression). In certain configurations, a spinner control used in an interface moves by 0.25 increments maximum Log 2 TPM values. In certain configurations, any number for maximum Log 2 TPM values can be manually entered by a user. Non-limiting examples of maximum Log 2 TPM values and/or TPM value thresholds (e.g., for non-diseased tissues) include −1, −0.5, 0, 0.5, and 1. Transcript expression level information can exist, for example, as average expression level values. For example, expression level information can exist as average TPM values, average FPKM values, average RPKM values, average RSEM values, or combination thereof. As used herein, an average value can be a mean, median, or mode value.

In some embodiments, one or more tables in a database comprise transcript expression values for non-diseased tissue samples. In some embodiments, non-diseased tissue samples were from multiple subjects different than the subjects from which diseased tissue samples were obtained. Non-diseased tissue sample transcript expression values may be obtained from any suitable source or dataset. In some embodiments, non-diseased tissue sample transcript expression values are obtained from a Genotype-Tissue Expression (GTEx) dataset. Without being limited by theory, this database feature allows for accurate identification of sub-populations of disease-associated transcripts due, in part, to a large population of baseline non-diseased sample transcript expression values. In some embodiments, non-diseased tissues comprise tissues chosen from adipose (e.g., subcutaneous, visceral (omentum)), adrenal gland, artery (e.g., aorta, coronary, tibial), brain (e.g., amygdala, anterior cingulate cortex (BA24), caudate (basal ganglia), cerebellar hemisphere, cerebellum, cortex, frontal cortex (BA9), hippocampus, hypothalamus, nucleus accumbens (basal ganglia), putamen (basal ganglia), spinal cord (cervical c-1), substantia nigra), breast (e.g., mammary tissue), colon (e.g., sigmoid, transverse), esophagus (e.g., gastroesophageal junction, mucosa, muscularis), heart (e.g., atrial appendage, left ventricle), kidney (e.g., cortex, medulla), liver, lung, minor salivary gland, muscle (e.g., skeletal), nerve (e.g., tibial), ovary, pancreas, pituitary, prostate, skin (e.g., not sun exposed (e.g., suprapubic), sun exposed (e.g., lower leg)), small intestine (e.g., terminal ileum), spleen, stomach, testis, thyroid, uterus, vagina, whole blood, bladder, cervix (e.g., endocervix, ectocervix), fallopian tube, and cells (e.g., EBV-transformed lymphocytes, cultured fibroblasts). In some embodiments, non-diseased tissues exclude cultured cells.

In some embodiments, one or more tables in a database comprise transcript expression values for diseased tissue samples. In some embodiments, diseased tissue samples were from multiple subjects different than the subjects from which non-diseased tissue samples were obtained. Diseased tissue sample transcript expression values may be obtained from any suitable source or dataset. In some embodiments, one or more tables comprise transcript expression values from diseased tissue from child subjects. In some embodiments, diseased tissue sample transcript expression values are obtained from the Therapeutically Applicable Research to Generate Effective Treatments (TARGET) dataset. In some embodiments, one or more tables comprise transcript expression values from diseased tissue from adult subjects. In some embodiments, diseased tissue sample transcript expression values are obtained from The Cancer Genome Atlas (TCGA) dataset. In some embodiments, one or more tables in a database comprise transcript expression values for diseased tissue samples from a TCGA database, and transcript expression values for non-diseased tissue samples from a GTEx database. In some database configurations, there is an intra- and an inter-dataset subject's independence. That is, subjects in each dataset (e.g., GTEx and TCGA) are independent (no overlapping), and TCGA and GTEx subjects are different (TCGA subjects are individuals diagnosed with a tumor, GTEx subjects are tumor-free). In some embodiments, one or more tables in a database comprise transcript expression values for non-diseased tissue adjacent to diseased tissue in the same subjects from the TCGA database. Accordingly, in certain database configurations, TCGA diseased tissue (e.g., tumor tissue) is compared to matched TCGA adjacent non-diseased tissue (i.e., normal tissue). In some embodiments, one or more tables in a database exclude transcript expression values for non-diseased tissue adjacent to diseased tissue in the same subjects from the TCGA database. Accordingly, in certain database configurations, TCGA diseased tissue (e.g., tumor tissue) is not compared to matched TCGA adjacent non-diseased tissue (i.e., normal tissue). TCGA adjacent non-diseased tissue (i.e., normal tissue) typically is sourced from diseased (e.g., cancer) patients. Such tissue may be subject to tumor field effects and/or may not match the tumor tissue of origin. For example, a tumor tissue of origin could be epithelial cells and adjacent normal tissue might include muscle or other tissue. Methods and systems herein generally comprise a highly tissue-specific transcript isoform analysis. In certain configurations, diseased samples are compared to multiple non-diseased samples from multiple tissues (e.g., about 7,400 non-diseased samples from about 47 tissues). Such configurations may provide a robust disease vs. non-disease (e.g., tumor vs. normal) transcript analysis, and may be useful for developing an immunotherapy that specifically targets cancer cells.

In some embodiments, transcript expression values in one or more tables have been processed according to a common gene model. In some embodiments, a common gene model is a single Gencode version (e.g., Gencode v23). A non-limiting example of a transcript expression value processor that uses a common gene model is TOIL. For example, the reprocessing of RNASeq data from TCGA, TARGET, and GTEX by the Xena group at UCSC using the TOIL pipeline provided a single transcript expression dataset constructed with a common gene model, Gencode v23 (see e.g., World Wide Web Uniform Resource Locator xenabrowser.net/datapages/?hub=https://toil.xenahubs.net:443). Without being limited by theory, this system structure may allow for accurate identification of transcripts in sub-populations as expression values for different transcripts have been normalized. This system structure also may allow for addition of transcript expression level values for diseased tissue samples, processed according to the same gene model already utilized for database generation, without requiring addition of comparative expression values in non-diseased samples. Accordingly, in some embodiments, a method herein comprises entering additional transcript expression level values for diseased tissue samples, where the expression level values have been processed according to the same gene model, without entering an expression level value for a comparative non-diseased sample.

In some embodiments, one or more tables in a database comprise transcript expression values for multiple transcripts. For example, one or more tables in a database may comprise transcript expression values for at least 10,000 transcripts, at least 20,000 transcripts, at least 30,000 transcripts, at least 40,000 transcripts, at least 50,000 transcripts, at least 60,000 transcripts, at least 70,000 transcripts, at least 80,000 transcripts, at least 90,000 transcripts, or at least 100,000 transcripts. In some embodiments, one or more tables in a database comprise transcript expression values for at least 50,000 transcripts.

In some embodiments, one or more tables in a database comprise transcript expression values for multiple genes. For example, one or more tables in a database may comprise transcript expression values for at least 1,000 genes, at least 5,000 genes, at least 10,000 genes, at least 15,000 genes, at least 16,000 genes, at least 17,000 genes, at least 18,000 genes, at least 19,000 genes, at least 20,000 genes, at least 21,000 genes, at least 22,000 genes, at least 23,000 genes, at least 24,000 genes, at least 25,000 genes, or at least 30,000 genes. In some embodiments, one or more tables in a database comprise transcript expression values for at least 19,000 genes.

In some embodiments, one or more tables in a database comprise transcript expression values for multiple diseased samples. For example, one or more tables in a database may comprise transcript expression values for at least 1,000 diseased samples, at least 2,000 diseased samples, at least 3,000 diseased samples, at least 4,000 diseased samples, at least 5,000 diseased samples, at least 6,000 diseased samples, at least 7,000 diseased samples, at least 8,000 diseased samples, at least 9,000 diseased samples, at least 10,000 diseased samples, at least 11,000 diseased samples, at least 12,000 diseased samples, at least 13,000 diseased samples, at least 14,000 diseased samples, at least 15,000 diseased samples, at least 16,000 diseased samples, at least 17,000 diseased samples, at least 18,000 diseased samples, at least 19,000 diseased samples, or at least 20,000 diseased samples. In some embodiments, one or more tables in a database comprise transcript expression values for at least 10,000 diseased samples.

In some embodiments, one or more tables in a database comprise transcript expression values for multiple non-diseased samples. For example, one or more tables in a database may comprise transcript expression values for at least 1,000 non-diseased samples, at least 2,000 non-diseased samples, at least 3,000 non-diseased samples, at least 4,000 non-diseased samples, at least 5,000 non-diseased samples, at least 6,000 non-diseased samples, at least 7,000 non-diseased samples, at least 8,000 non-diseased samples, at least 9,000 non-diseased samples, at least 10,000 non-diseased samples, at least 11,000 non-diseased samples, at least 12,000 non-diseased samples, at least 13,000 non-diseased samples, at least 14,000 non-diseased samples, at least 15,000 non-diseased samples, at least 16,000 non-diseased samples, at least 17,000 non-diseased samples, at least 18,000 non-diseased samples, at least 19,000 non-diseased samples, or at least 20,000 non-diseased samples. In some embodiments, one or more tables in a database comprise transcript expression values for at least 7,000 non-diseased samples.

In some embodiments, one or more tables in a database comprise transcript expression values for multiple defined diseases. For example, one or more tables in a database may comprise transcript expression values for at least 20 defined diseases, at least 25 defined diseases, at least 30 defined diseases, at least 31 defined diseases, at least 32 defined diseases, at least 33 defined diseases, at least 34 defined diseases, at least 35 defined diseases, at least 36 defined diseases, at least 37 defined diseases, at least 38 defined diseases, at least 39 defined diseases, at least 40 defined diseases, at least 45 defined diseases, or at least 50 defined diseases. In some embodiments, one or more tables in a database comprise transcript expression values for at least 35 defined diseases.

In some embodiments, one or more tables in a database comprise transcript expression values for multiple non-diseased tissues. For example, one or more tables in a database may comprise transcript expression values for at least 30 non-diseased tissues, at least 35 non-diseased tissues, at least 40 non-diseased tissues, at least 41 non-diseased tissues, at least 42 non-diseased tissues, at least 43 non-diseased tissues, at least 44 non-diseased tissues, at least 45 non-diseased tissues, at least 46 non-diseased tissues, at least 47 non-diseased tissues, at least 48 non-diseased tissues, at least 49 non-diseased tissues, at least 50 non-diseased tissues, at least 55 non-diseased tissues, or at least 60 non-diseased tissues. In some embodiments, one or more tables in a database comprise transcript expression values for at least 45 non-diseased tissues.

In some embodiments, one or more tables in a database comprise transcript expression values for multiple transcripts for multiple genes, multiple diseased samples, multiple non-diseased samples, multiple defined diseases, and multiple non-diseased tissues. In some embodiments, one or more tables in a database comprise transcript expression values for at least 50,000 transcripts for at least 19,000 genes, at least 10,000 diseased samples, at least 7,000 non-diseased samples, at least 35 defined diseases and 45 non-diseased tissues.

In some embodiments, a database comprises one or more precomputed features or values. A precomputed feature or value may be referred to as a pre-calculated feature or value, a pre-populated feature or value, a data bundle, a data bin, precomputed binned data, precomputed binned threshold, and the like. A feature or value may be precomputed during construction of a database such that when a user accesses the database, the precomputed feature or value is already present in the database. Without being limited by theory, rapid identification of transcripts and/or sub-populations of transcripts across large numbers of transcripts is due, in part, to the presence of precomputed features and/or values. A database that includes precomputed features or values may require less memory and provide faster search results compared to a database without precomputed features or values (e.g., a database comprising raw data). In some embodiments, for each transcript (e.g., each known protein coding transcript), a database comprises a precomputed maximum expression for non-diseased samples. For example, a database may comprise a box plot upper whisker TPM value in non-diseased tissues for each transcript. In some embodiments, for each transcript (e.g., each known protein coding transcript), a database comprises a precomputed median expression (e.g., median TPM expression value) for non-diseased samples. In some embodiments, for each transcript (e.g., each known protein coding transcript), a database comprises a plurality of precomputed minimum transcript expression value thresholds (e.g., TPM expression value thresholds) for diseased tissue samples. In some embodiments, for each transcript (e.g., each known protein coding transcript), a database comprises a percentage of samples exceeding predefined expression levels (e.g., TPM expression levels) in diseased tissue. In some embodiments, for each transcript (e.g., each known protein coding transcript), a database comprises an average expression (e.g., average TPM expression) in diseased tissue for each of the predefined expression levels (e.g., TPM expression levels). In some embodiments, for each transcript (e.g., each known protein coding transcript), a database comprises a plurality of precomputed transcript expression ratio thresholds (e.g., for transcript expression ratios generated according to transcript expression values for diseased tissue samples and transcript expression values for non-diseased tissues).

A method herein may comprise generating a database. A database may be generated by importing one or more datasets. A database may be generated by importing one or more datasets comprising transcript expression data, transcript isoform expression data, RNASeq data, and/or other transcript/protein data. Datasets may include, for example, datasets from data sources (e.g., The Cancer Genome Atlas (TCGA), Therapeutically Applicable Research to Generate Effective Treatments (TARGET), Genotype-Tissue Expression (GTEx), TOIL, Gencode (e.g., Gencode v23), a surfaceome dataset, and Matched Annotation from the NCBI and EBI (MANE)). A database may be generated by relating two or more features described above. A database may be generated by importing one or more tables that relate two or more features described herein. For example, a method for generating a database may comprise one or more of (i) relating transcript identifiers to genes, where at least a portion of the transcript identifiers correspond to transcript isoform sets, and each of the transcript isoform sets is encoded by a gene; (ii) relating transcript identifiers to corresponding transcript expression values in diseased tissue samples for a plurality of defined diseases, and (iii) relating percentages of diseased tissue samples for each defined disease to corresponding transcript identifiers, where the percentages are based on an amount of diseased tissue samples corresponding to a defined disease for which transcript expression values of a corresponding transcript identifier exceed a defined minimum transcript expression value threshold. In certain configurations, a method for generating a database may comprise one or more of (i) relating transcript identifiers to genes, where at least a portion of the transcript identifiers correspond to transcript isoform sets, and each of the transcript isoform sets is encoded by a gene; (ii) relating transcript identifiers to corresponding transcript expression values in diseased tissue samples for a plurality of defined diseases, and (iii) relating percentages of diseased tissue samples for each defined disease to corresponding transcript identifiers, where the percentages are based on an amount of diseased tissue samples corresponding to a defined disease for which transcript expression ratios of a corresponding transcript identifier exceed a defined transcript expression ratio threshold. An example method for generating a database is provided in Example 1.

Input

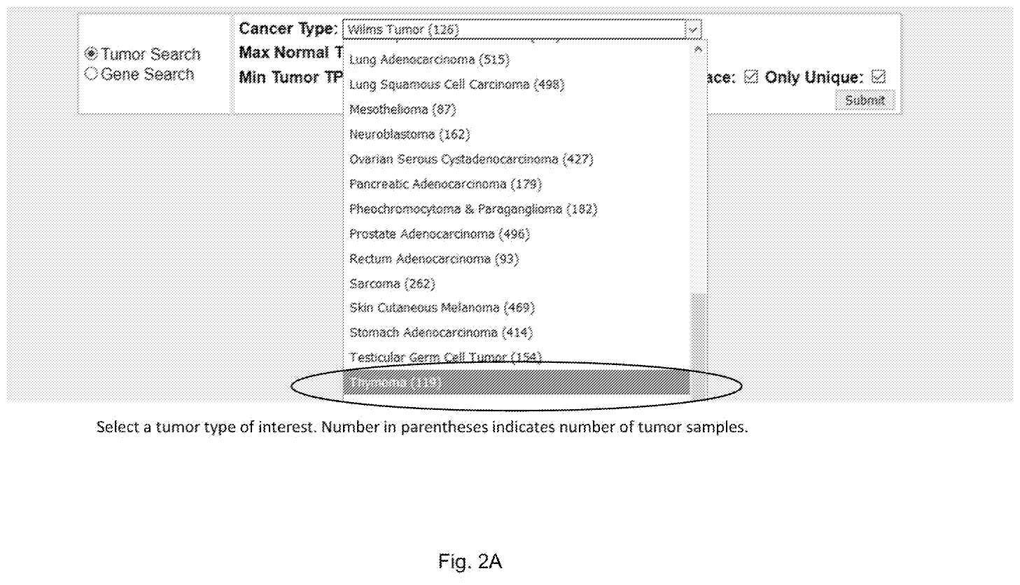

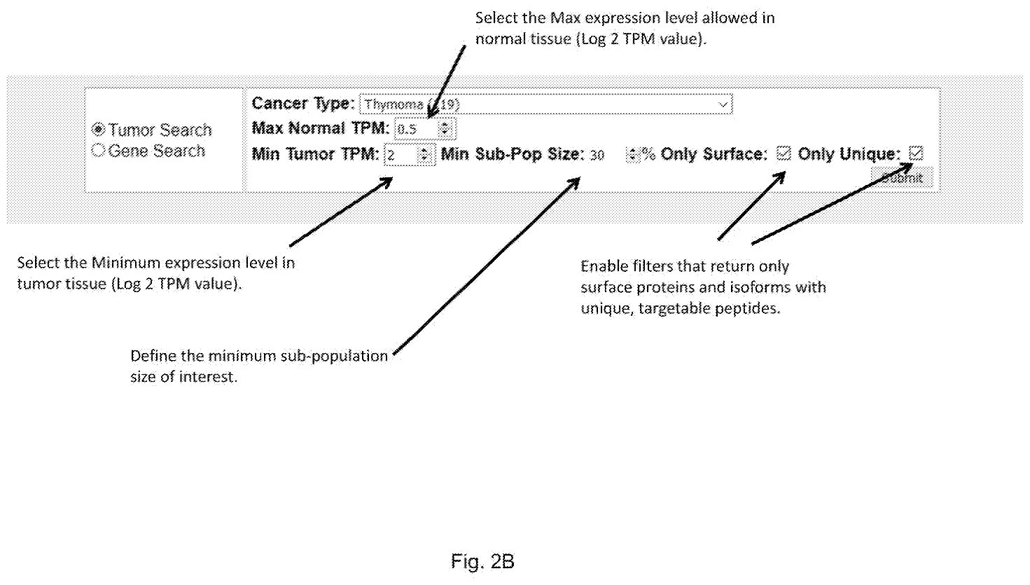

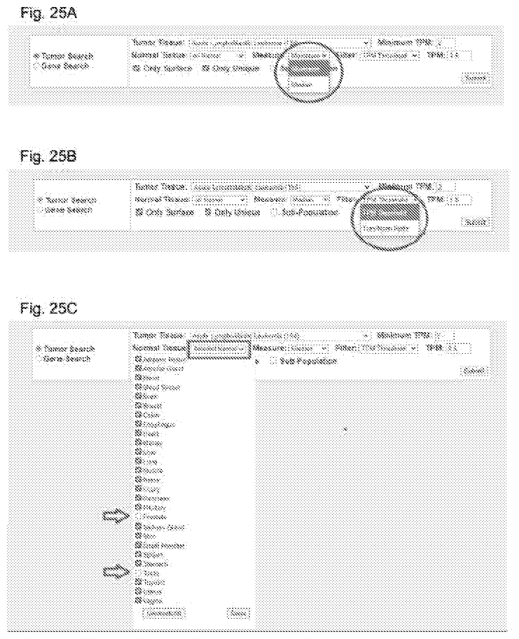

In some embodiments, a method herein comprises receiving user input. User input may be received by way of an interface (e.g., a graphical user interface). Examples of a user input interface is provided in FIG. 2A, FIG. 2B, FIG. 25A, FIG. 25B, and FIG. 25C. User input may include one or more defined features selected by a user for searching a database. The term defined features refers to feature options that are prepopulated in a database. In some embodiments, a defined feature is a defined disease. A defined disease may be selected from a plurality of defined diseases (e.g., a plurality of defined diseases described herein). Accordingly, in some embodiments, one defined disease of a plurality of defined diseases is received as input. In some embodiments, a defined feature is multiple defined diseases. Accordingly, in some embodiments, multiple defined diseases of a plurality of defined diseases are received as input. In some embodiments, a defined feature is all defined diseases. Accordingly, in some embodiments, all defined diseases of a plurality of defined diseases are received as input. In such instances, this system structure allows for rapid identification of sub-populations of transcripts overexpressed in only a small number of diseased tissue samples per disease. Without use of the methods and systems described herein, such transcripts typically are not readily identified in a number of diseases.

Each of the defined diseases may correspond to diseased tissue samples (e.g., tissue samples from subjects having, or suspected of having, the defined diseases). Non-limiting examples of defined diseases include Alzheimer's disease, Parkinson's disease, Lupus, IPEX syndrome, diabetes, rheumatoid arthritis, influenza, pneumonia, tuberculosis, acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, chronic myeloid leukemia, acute lymphocytic leukemia, multiple myeloma, non-Hodgkin lymphoma, Hodgkin lymphoma, marginal zone lymphoma, follicular lymphoma, small lymphocyte lymphoma, B-cell lymphoma, diffuse large B-cell lymphoma or mantle cell lymphoma, acute lymphoblastic leukemia, adrenocortical cancer, cervical and endocervical cancer, clear cell sarcoma of the kidney, kidney clear cell carcinoma, kidney papillary cell carcinoma, neuroblastoma, testicular germ cell tumor, uterine corpus endometrioid carcinoma, Wilms tumor, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like.

In some embodiments, defined diseases comprise defined cancers. Defined cancers may be chosen from acute lymphoblastic leukemia, acute myeloid leukemia, adrenocortical cancer, bladder urothelial carcinoma, brain lower grade glioma, breast invasive carcinoma, cervical & endocervical cancer, cholangiocarcinoma, clear cell sarcoma of the kidney, colon adenocarcinoma, diffuse large B-cell lymphoma, esophageal carcinoma, glioblastoma multiforme, head & neck squamous cell carcinoma, kidney chromophobe, kidney clear cell carcinoma, kidney papillary cell carcinoma, liver hepatocellular carcinoma, lung adenocarcinoma, lung squamous cell carcinoma, mesothelioma, neuroblastoma, ovarian serous cystadenocarcinoma, pancreatic adenocarcinoma, pheochromocytoma & paraganglioma, prostate adenocarcinoma, rectum adenocarcinoma, sarcoma, skin cutaneous melanoma, stomach adenocarcinoma, testicular germ cell tumor, thymoma, thyroid carcinoma, uterine carcinosarcoma, uterine corpus endometrioid carcinoma, uveal melanoma, and Wilms tumor.

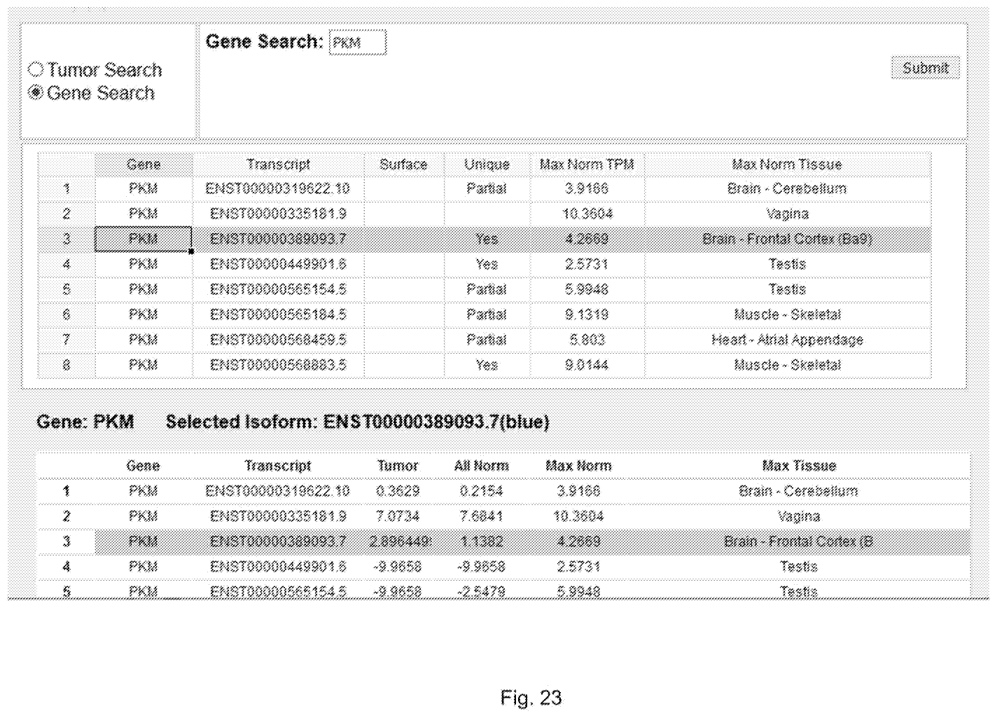

In some embodiments, a defined feature is a gene. Accordingly, in some embodiments, a defined gene of a plurality of defined genes is received as input (see e.g., FIG. 23). By selecting a defined gene, a user can identify whether elevated transcript expression exists for one or more transcript isoforms of the gene for one or more defined diseases (e.g., by scanning an outputted list for transcripts that are high in diseased tissue and low in all non-diseased tissues or a subset of non-diseased tissues). In some instances, a plurality of defined diseases having elevated transcript expression is identified. In some instances, one or more of the defined diseases was known to have elevated transcript expression, and one or more of the defined diseases was not previously known to have elevated transcript expression. In such instances, one or more new indications for a known transcript isoform may be identified.

In some embodiments, a defined feature is a defined minimum transcript expression value threshold for diseased tissue samples. In some embodiments, a defined minimum transcript expression value threshold for diseased tissue samples is selected from a plurality of defined minimum transcript expression value thresholds for diseased tissue samples. In some embodiments, a database comprises a plurality of precomputed minimum transcript expression value thresholds for diseased tissue samples, and a user selects one of the precomputed minimum transcript expression value thresholds. Precomputed minimum transcript expression value thresholds may be referred to as precomputed binned thresholds for diseased tissues.

In some embodiments, a defined feature is a defined maximum transcript expression value for non-diseased tissues. For non-diseased or normal tissues, a database may include statistical summarizations of normal tissues such as, for example, high value, low value, 25 percentile, mean, 75th percentile, and outliers. In addition, a database may summarize for each transcript the highest value seen in any normal tissue and identifies the tissue. In some embodiments, a database may include for each transcript, a precomputed box plot upper whisker TPM value in non-diseased tissues, sometimes referred to as max normal expression. In some embodiments, a defined maximum transcript expression value threshold for non-diseased tissues is selected from a plurality of defined maximum transcript expression value thresholds for non-diseased tissues. In some embodiments, a database comprises a plurality of precomputed maximum transcript expression value thresholds for non-diseased tissue samples, and a user selects one of the precomputed maximum transcript expression value thresholds. Precomputed maximum transcript expression value thresholds may be referred to as precomputed binned thresholds for non-diseased tissues. In some embodiments, a database does not comprise precomputed maximum transcript expression value thresholds for non-diseased tissue samples, and a user selects or enters a maximum transcript expression value for non-diseased tissues.

In some embodiments, a defined feature is a defined median transcript expression value for non-diseased tissues. A median transcript expression value generally refers to the median (or middle value separating the higher half from the lower half of a data set) expression value for all (or a subset of) non-diseased tissues used in the database. Generally, a median is the middle value when all values are put in sequential order. If there are an even number of values, it is the average of the two values closest to the middle. In some embodiments, the median expression value is a median of the mean values in a non-diseased tissues expression database (e.g., GTEx). In certain configurations, an application with certain user settings compares median tumor value for a selected tumor type to the highest median value of the normal tissues.

In some embodiments, a defined median transcript expression value threshold for non-diseased tissues is selected from a plurality of defined median transcript expression value thresholds for non-diseased tissues. In some embodiments, a database comprises a plurality of precomputed median transcript expression value thresholds for non-diseased tissue samples, and a user selects one of the precomputed median transcript expression value thresholds. Precomputed median transcript expression value thresholds may be referred to as precomputed binned thresholds for non-diseased tissues. In some embodiments, a database does not comprise precomputed median transcript expression value thresholds for non-diseased tissue samples, and a user selects or enters a median transcript expression value for non-diseased tissues.

In some embodiments, a defined feature is a defined transcript expression ratio threshold. A defined transcript expression ratio threshold may also be referred to as a defined transcript expression ratio value threshold. A defined transcript expression ratio threshold generally is generated according to transcript expression values for diseased tissue samples and transcript expression values for non-diseased tissues. In certain configurations, a defined transcript expression ratio threshold is a ratio of a transcript expression value for diseased tissue samples to a transcript expression value for non-diseased tissues. In some embodiments, a defined transcript expression ratio threshold is selected from a plurality of defined transcript expression ratio thresholds. In some embodiments, a database comprises a plurality of precomputed defined transcript expression ratio thresholds generated according to expression data for diseased and non-diseased tissue samples, and a user selects one of the precomputed transcript expression ratio thresholds. Precomputed transcript expression ratio thresholds may be referred to as precomputed binned ratio thresholds. In certain configurations, a ratio is of mean or median diseased tissue expression to non-diseased tissue expression, where the non-diseased tissue expression is the expression value that shows the highest mean among the tissues of choice. In certain configurations, one or more non-diseased tissues may be excluded from this calculation, for example the testis, and/or other tissues considered non-vital or irrelevant. In certain configurations, a ratio is a TPM tumor value divided by a TPM non-disease value. For example, when a user selects ‘maximum’ for non-diseased tissue, the ratio is of the median of the selected tumor type divided by the maximum TPM value observed in any of the non-diseased tissues. When a user selects ‘median’ for non-diseased tissue, the ratio is of the median of the selected tumor type divided by the highest median TPM value observed in any non-diseased tissue. The same logic outlined above is true if a subpopulation is specified except that the numerator is the value from the subset of tumor samples that meet the subpopulation criterion as described herein.

In some embodiments, average transcript expression levels for diseased tissue samples can be calculated after a defined minimum transcript expression value threshold is received. In some embodiments, average transcript expression levels for diseased tissue samples can be calculated before a defined minimum transcript expression value threshold is received. In some embodiments, a database is pre-populated with average transcript expression levels calculated for each of the defined minimum expression thresholds. Without being limited by theory, rapid identification of sub-populations of transcripts across large numbers of transcripts is due, in part, to pre-population of calculated average transcript expression for an input defined disease for each minimum expression threshold. In some embodiments, average transcript expression levels for diseased tissue samples can be calculated after a defined transcript expression ratio threshold is received. In some embodiments, average transcript expression levels for diseased tissue samples can be calculated before a defined transcript expression ratio threshold is received. In some embodiments, a database is pre-populated with average transcript expression levels calculated for each of the defined transcript expression ratio thresholds. Without being limited by theory, rapid identification of sub-populations of transcripts across large numbers of transcripts is due, in part, to pre-population of calculated average transcript expression for an input defined disease for each transcript expression ratio threshold. In certain configurations, for diseased samples, a precompute is done for each of the possible threshold values (1 to 10, in increments of 1). At each threshold level, the percentage of samples that exceed the threshold is tallied. This supports fast target searches with a reasonable size database including subpopulation searches. In certain configurations, high value, low value, 25th percentile, mean, 75th percentile and outliers for each transcript for each disease type is computed and/or precomputed. In certain configurations, the percentage of samples that exceed a threshold for each possible disease TPM threshold for every transcript is computed and/or precomputed.

In some embodiments, average transcript expression levels for non-diseased tissue samples can be calculated after a defined maximum transcript expression value threshold is received. In some embodiments, average transcript expression levels for non-diseased tissue samples can be calculated before a defined maximum transcript expression value threshold is received. In some embodiments, a database is pre-populated with average transcript expression levels calculated for each of the defined maximum expression thresholds. In some embodiments, a database is not pre-populated with average transcript expression levels calculated for maximum expression values.

In some embodiments, average transcript expression levels for non-diseased tissue samples can be calculated after a defined median transcript expression value threshold is received. In some embodiments, average transcript expression levels for non-diseased tissue samples can be calculated before a defined median transcript expression value threshold is received. In some embodiments, a database is pre-populated with average transcript expression levels calculated for each of the defined median expression thresholds. In some embodiments, a database is not pre-populated with average transcript expression levels calculated for median expression values.

In some embodiments, one or more tables in a database relate average transcript expression values to corresponding transcript identifiers for each defined disease. Accordingly, for a defined disease, a user can obtain average transcript expression values for one or more transcripts. In some embodiments, each of the average transcript expression values is calculated based on transcript expression values for diseased tissue samples for which the transcript expression values exceed a defined minimum transcript expression value threshold. In some embodiments, each of the average transcript expression values is calculated for each defined minimum transcript expression value threshold of a plurality of defined minimum transcript expression value thresholds. In some embodiments, each of the average transcript expression values is precomputed for each defined minimum transcript expression value threshold of a plurality of defined minimum transcript expression value thresholds. In some embodiments, each of the average transcript expression values is calculated based on transcript expression values for diseased tissue samples for which the transcript expression values exceed a defined transcript expression ratio threshold. In some embodiments, each of the average transcript expression values is calculated for each defined transcript expression ratio threshold of a plurality of defined transcript expression ratio thresholds.

In some embodiments, each of the average transcript expression values is precomputed for each defined transcript expression ratio threshold of a plurality of defined transcript expression ratio thresholds. In some embodiments, a list of outputted transcripts for a defined disease comprises an average transcript expression value for each of the transcript identifiers listed. An average transcript expression value may be a mean transcript expression value. An average transcript expression value may be a median transcript expression value.

In some embodiments, a defined feature is a defined minimum sample sub-population percentage. In some embodiments, a defined minimum sample sub-population percentage is selected from a plurality of defined minimum sample sub-population percentages. In some embodiments, a database comprises a plurality of precomputed minimum sample sub-population percentages, and a user selects one of the precomputed minimum sample sub-population percentages. A defined minimum sample sub-population percentage can range from about 1% to about 100%. For example, a defined minimum sample sub-population percentage may be about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%. In some embodiments, a defined minimum sample sub-population percentage can range from about 10% to about 40%. For example, a defined minimum sample sub-population percentage may be about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, one or more tables in a database relate percentages of diseased tissue samples to corresponding transcript identifiers for each defined disease. Accordingly, for a defined disease, and for a defined minimum expression value in diseased tissue samples, a user can select a percentage of diseased tissue samples and obtain a list of transcripts that have at least the selected minimum expression value in the selected percentage of diseased samples. In some embodiments, for a defined disease, for a defined minimum expression value in diseased tissue samples, and for a defined maximum or defined median expression value in non-diseased tissue, a user can select a percentage of diseased tissue samples and obtain a list of transcripts that have at least the selected minimum expression value (and the selected maximum or selected median expression value in non-diseased tissue) in the selected percentage of diseased samples. In certain configurations, for a defined disease, and for a defined transcript expression ratio threshold, a user can select a percentage of diseased tissue samples and obtain a list of transcripts that have at least the selected transcript expression ratio threshold in the selected percentage of diseased samples. A percentage of diseased samples can range from about 1% to about 100%. For example, about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of diseased samples may be selected. In some embodiments, percentage of diseased samples can range from about 10% to about 40%. For example, about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of diseased samples may be selected.

In some embodiments, each of the percentages is calculated based on an amount of diseased tissue samples for which the transcript expression values of a corresponding transcript identifier exceed the defined minimum transcript expression value threshold. In some embodiments, each of the percentages is calculated for each defined minimum transcript expression value threshold of the plurality of defined minimum transcript expression value thresholds. In some embodiments, each of the percentages equals (i) the number of diseased samples for a defined disease for which transcript expression values for a corresponding transcript identifier exceeds the defined minimum transcript expression value threshold, divided by (ii) total number of diseased samples for the defined disease. In some embodiments, a list of outputted transcript identifiers comprises a percentage of samples exceeding defined minimum transcript expression value threshold for each of the transcript identifiers listed. In some embodiments, a database is pre-populated with diseased sample percentages calculated for each of the defined minimum expression thresholds. Without being limited by theory, rapid identification of sub-populations of transcripts across large numbers of transcripts is due, in part, to pre-population of calculated percentages for each minimum expression threshold.

In some embodiments, each of the percentages is calculated based on an amount of diseased tissue samples for which the transcript expression ratios of a corresponding transcript identifier exceed the defined transcript expression ratio threshold. In some embodiments, each of the percentages is calculated for each defined transcript expression ratio threshold of the plurality of defined transcript expression ratio thresholds. In some embodiments, each of the percentages equals (i) the number of diseased samples for a defined disease for which transcript expression ratio values for a corresponding transcript identifier exceeds the defined transcript expression ratio threshold, divided by (ii) total number of diseased samples for the defined disease. In some embodiments, a list of outputted transcript identifiers comprises a percentage of samples exceeding a defined transcript expression ratio threshold for each of the transcript identifiers listed. In some embodiments, a database is pre-populated with diseased sample percentages calculated for each of the defined transcript expression ratio thresholds. Without being limited by theory, rapid identification of sub-populations of transcripts across large numbers of transcripts is due, in part, to pre-population of calculated percentages for each transcript expression ratio threshold.

In some embodiments, a method herein comprises identifying one or more transcript identifiers meeting certain criteria. In some embodiments, a method herein comprises identifying one or more transcript identifiers meeting certain criteria for a percentage of diseased samples greater than a defined minimum sub-population percentage. In some embodiments, a method herein comprises identifying one or more transcript identifiers meeting certain criteria for a percentage of diseased samples corresponding to a defined disease greater than a defined minimum sub-population percentage. A percentage of diseased samples can be calculated after the defined minimum sample sub-population percentage is received, or before it is received (e.g., the percentage is precomputed). Without being limited by theory, a system structure described herein allows for rapid identification of sub-populations of transcripts according to a percentage threshold. One or more transcript identifiers may be identified in a database (e.g., a database described herein). Criteria for identifying one or more transcript identifiers may include a corresponding transcript expression value in diseased tissue samples (corresponding to an input defined disease) that is greater than an input defined minimum transcript expression value threshold, and/or a corresponding transcript expression value in non-diseased tissue that is less than an input defined maximum or defined median transcript expression value threshold. In certain configurations, criteria for identifying one or more transcript identifiers may include a corresponding transcript expression ratio value (corresponding to an input defined disease) that is greater than an input defined transcript expression ratio threshold.

In some embodiments, a method herein comprises receiving user input where one or more filters is selected. For example, a user may select a cell surface filter. Selection of a cell surface filter allows for rapid identification of transcripts that can be targeted (e.g., by a binding molecule not requiring cellular uptake, for development of a therapy or diagnostic). In some embodiments, one or more tables in a database described herein comprise a transcript table. A transcript table can identify a subset of gene identifiers each corresponding to a gene encoding a cell surface protein. User input may comprise selecting a filter for outputting transcript identifiers corresponding to genes encoding cell surface proteins. Accordingly, a method herein may comprise identifying one or more transcript identifiers corresponding to genes encoding cell surface proteins.

In some embodiments, a user may select a unique polypeptide filter. Selection of a unique polypeptide filter allows for rapid identification of transcripts that can be targeted with minimized off-target effects (e.g., by a binding molecule with enhanced specificity for a particular target, for development of a therapy or diagnostic). In some embodiments, one or more tables in a database described herein comprise a transcript table. A transcript table can identify a subset of transcript identifiers each corresponding to a transcript encoding a unique polypeptide comprising an insertion, deletion or substitution of one or more amino acids relative to polypeptides encoded by other transcript isoforms of the same gene (e.g., encoded by a canonical isoform). Accordingly, a method herein may comprise identifying one or more transcript identifiers corresponding to transcripts encoding unique polypeptides.

In some embodiments, a user may select a unique or partially unique polypeptide filter. Such filter would identify transcripts that encode a unique polypeptide as described above and transcripts that encode a partially unique polypeptide. A partially unique polypeptide refers to a polypeptide encoded by more than one, but not all, transcript isoforms for a select gene. Selection of a unique/partially unique polypeptide filter allows for rapid identification of transcripts that can be targeted with some but not all possible off-target effects. In some embodiments, a transcript table can identify a subset of transcript identifiers each corresponding to transcripts encoding a partially unique polypeptide comprising an insertion, deletion or substitution of one or more amino acids relative to polypeptides encoded by some of the other transcript isoforms of the same gene (e.g., encoded by a canonical isoform). Accordingly, a method herein may comprise identifying one or more transcript identifiers corresponding to transcripts encoding partially unique polypeptides. In some embodiments, user input comprises selection of a function for merging the expression values for transcripts encoding partially unique polypeptides, thereby generating merged transcript expression values. In certain configurations, transcript expression values are merged among non-diseased tissues, but not diseased tissues. In certain configurations, transcript expression values are merged among non-diseased tissues, and are not merged for diseased tissues. In combining expression values for partially unique transcript isoforms, an example approach is to take a high percentile (e.g., the highest 70^{th}, 75^{th} or 80^{th} percentile) and whisker high value, average the medians, and take a low percentile (e.g., the lowest 20^{th}, 25^{th} or 30^{th} percentile) and whisker low value.

In some embodiments, user input comprises selecting all non-diseased tissues. For example, a user may select all non-diseased tissues (i.e., all non-diseased tissues for which sample data is included in a database described herein) when selecting a defined maximum or defined median transcript expression value threshold. In some embodiments, user input comprises selecting a subset of non-diseased tissues. For example, a user may select a subset of non-diseased tissues (i.e., a subset of non-diseased tissues for which sample data is included in a database described herein) when selecting a defined maximum or defined median transcript expression value threshold. In certain configurations, all non-diseased tissues are selected as a default and a user de-selects one or more non-diseased tissues. For example, a user interested in diseases of the female reproductive tract may de-select male-specific tissues such as testis or prostate. In some embodiments, a defined maximum transcript expression value threshold or a defined median transcript expression value threshold for non-diseased tissues is determined according to a subset of non-diseased tissues. In some embodiments, a defined transcript expression ratio threshold for diseased tissues vs. non-diseased tissues is determined, in part, according to a subset of non-diseased tissues. In some embodiments, a method herein comprises identifying in a database one or more transcript identifiers meeting the following criteria (1) and (2): (1) a corresponding transcript expression value in diseased tissue samples corresponding to an input defined disease is greater than an input defined minimum transcript expression value threshold, and (2) a corresponding transcript expression value in a subset of non-diseased tissues is less than an input defined maximum or defined median transcript expression value threshold.

Output

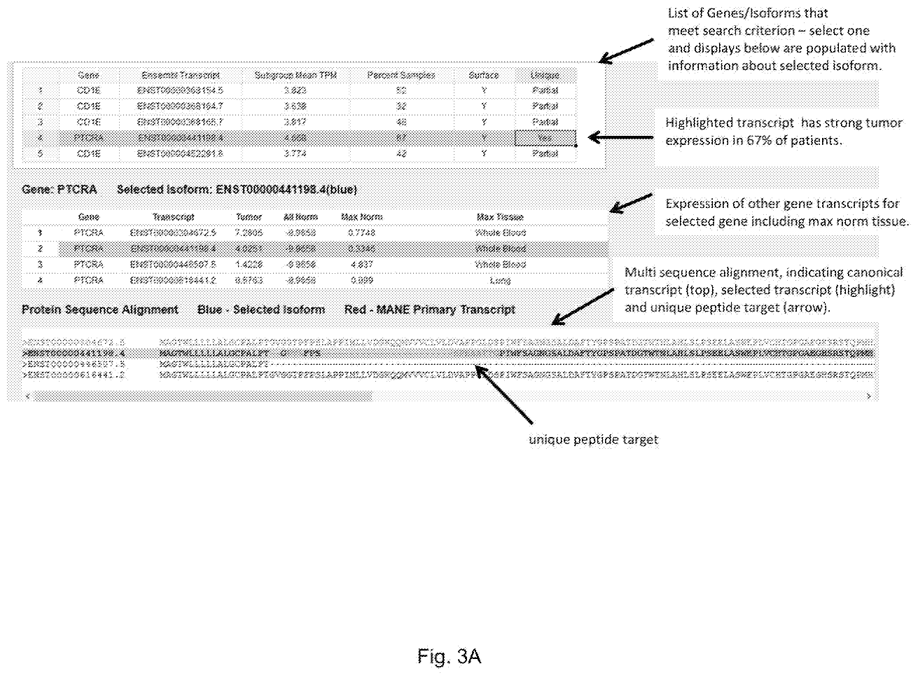

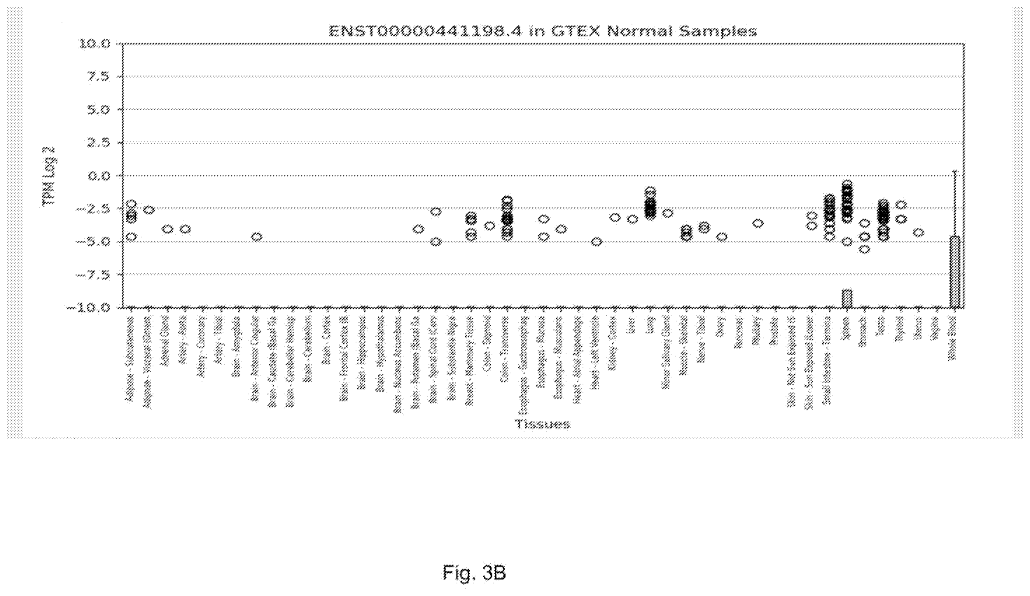

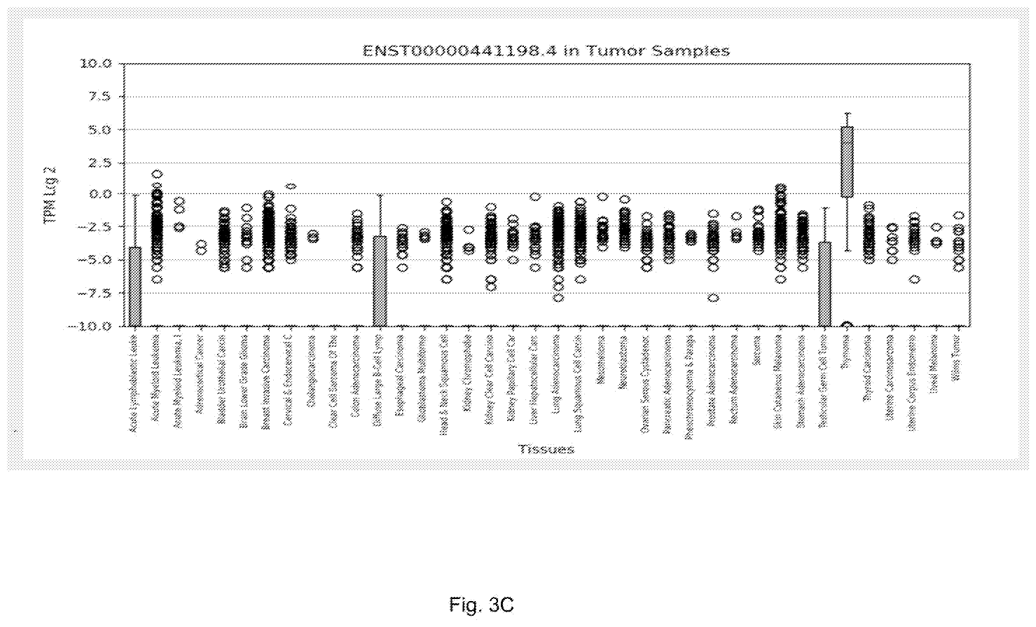

In some embodiments, a method or system herein comprises outputted information. Outputted information may be provided on a suitable visual medium (e.g., a peripheral or component of a machine, e.g., a printer, monitor, tablet, smart phone, display, and the like). Generally, outputted information can be displayed in a suitable format that facilitates analysis of one or more transcripts and/or expression values. Non-limiting examples of formats suitable for use for displaying outputted information include digital data, a graph, a 2D graph, a 3D graph, and 4D graph, a picture (e.g., a jpg, bitmap (e.g., bmp), pdf, tiff, gif, raw, png, the like or suitable format), a pictograph, a list, a sequence alignment, a chart, a table, a box plot, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, and combination of the foregoing. Outputted information may include one or more features described herein. An example output display is provided in FIGS. 3A-3C.

In some embodiments, a method herein comprises outputting a list of one or more transcript identifiers. In some embodiments, a list comprises a transcript identifier for each of one or more transcripts identified according to a method described herein (e.g., criteria described herein). In some embodiments, a transcript of one or more genes is identified that is expressed at a level in a defined subpopulation of diseased tissue samples higher than a level the transcript is expressed in non-diseased tissue samples.

An example process for identifying one or more transcript identifiers meeting certain criteria is as follows: an interface (e.g., HTML interface) runs a script (e.g., Javascript) to send user specified parameters for disease type (e.g., tumor type), percentage of samples, and expression thresholds to a code (e.g., Python code) run by a local webserver. The code (e.g., Python code) performs a relational query (e.g., SQL query) of a database (e.g., a database described herein) using the supplied parameters. The database build process previously precomputed for each known protein coding transcript: box plot upper whisker TPM value in non-diseased tissues (max normal expression), % of samples exceeding predefined expression levels in diseased tissue (e.g., tumor tissue), and average TPM expression in diseased tissue (e.g., tumor tissue) for each predefined TPM expression levels. Because of this precompute, it is a simple query (e.g., SQL query) to retrieve all transcripts which DO NOT have expression in non-diseased tissue above a user threshold but which DO have expression in disease tissue (tumor tissue) above a threshold for at least the specified number (percentage) of samples. The code (e.g., Python code) returns a list of transcripts meeting user criteria along with the percentage of samples and average expression of subpopulation above threshold for each.

In some embodiments, a method herein comprises receiving a user selection of a transcript identifier. For example, a user may select a transcript identifier outputted as described above. In some embodiments, a user selects a transcript identifier from a list of transcript identifiers outputted as described above (see e.g., FIG. 3A, top panel). After receiving a user selection of a transcript identifier, a method herein may comprise outputting a transcript identifier for each of one or more transcript isoforms encoded by the gene that encodes the transcript corresponding to the selected transcript identifier (see e.g., FIG. 3A, middle panel). The one or more transcript isoforms corresponding to the outputted transcript identifiers may be referred to as a transcript isoform set. In some embodiments, a method herein comprises, for each transcript identifier outputted, outputting an average transcript expression value in diseased tissue samples corresponding to an input defined disease. In some embodiments, a method herein comprises, for each transcript identifier outputted, outputting an average transcript expression value for all non-diseased tissue samples (e.g., all non-diseased tissue samples in a dataset or database) or a subset of non-diseased tissue samples (e.g., a subset of non-diseased tissue samples in a dataset or database).

In some embodiments, a method herein comprises, for each transcript identifier outputted, outputting a maximum transcript expression value of non-diseased samples (e.g., for a non-diseased tissue having the highest transcript expression value). In some embodiments, a method herein comprises, for each transcript identifier outputted, outputting a median transcript expression value of non-diseased samples (e.g., for a non-diseased tissue having the median transcript expression value). In some embodiments, a method herein comprises, for each transcript identifier outputted, outputting a non-diseased tissue corresponding to the maximum transcript expression value. In some embodiments, a method herein comprises, for each transcript identifier outputted, outputting a non-diseased tissue corresponding to the median transcript expression value. In some embodiments, one or more tables in a database described herein relate average transcript expression values in diseased tissue samples to corresponding transcript identifiers. In some embodiments, one or more tables in a database described herein relate average transcript expression values for all non-diseased samples or a subset of non-diseased samples to corresponding transcript identifiers. An average transcript expression value may be a median transcript expression value. An average transcript expression value may be a mean transcript expression value. In some embodiments, one or more tables in a database described herein relate maximum or median transcript expression values of non-diseased samples to corresponding transcript identifiers. In some embodiments, one or more tables in a database described herein relate non-diseased tissues corresponding to the maximum or median transcript expression values to corresponding transcript identifiers.

In some embodiments, a method herein comprises outputting an alignment of polypeptide linear sequences corresponding to the outputted transcript identifiers described above (see e.g., FIG. 3A, bottom panel). In some embodiments, one or more tables in a database described herein relate polypeptide linear sequences to corresponding transcript identifiers. In some embodiments, a database described herein comprises one or more tables comprising a transcript table. In some embodiments, a transcript table identifies a subset of transcript identifiers each as a primary transcript having an expression value (e.g., general expression value according to one or more data sources (e.g., MANE)) higher than the expression values corresponding to other transcript isoforms encoded by the same gene. A primary transcript may be referred to as a canonical transcript. In some embodiments, a method herein comprises outputting a primary/canonical transcript (e.g., within an isoform set). In some embodiments, when outputted, transcript identifiers identified as a primary transcript are ranked higher (e.g., listed first) relative to transcript identifiers corresponding to other transcript isoforms encoded by the same gene (see e.g., FIG. 3A, middle and bottom panels).

In some embodiments, a method herein comprises outputting one or more statistical representations of transcript expression values corresponding to a selected transcript identifier. In some embodiments, a method herein comprises outputting one or more box plots for a transcript corresponding to a selected transcript identifier (see e.g., FIG. 3B and FIG. 3C). In some embodiments, a box plot displays transcript expression values corresponding to a selected transcript identifier in diseased samples for each of a plurality of defined diseases (see e.g., FIG. 3C). In some embodiments, a box plot displays transcript expression values corresponding to a selected transcript identifier in non-diseased samples for each of a plurality of tissues (see e.g., FIG. 3B). Box plot statistics may comprise, for example, one or more of: average transcript expression value, first quantile of average transcript expression value, third quantile of average transcript expression value, maximum transcript expression value whisker, minimum transcript expression value whisker, and outlier designations for transcripts of samples in each tissue. An average transcript expression value may be a mean transcript expression value. An average transcript expression value may be a median transcript expression value. In some embodiments, one or more tables in a database described herein relate transcript expression values and box plot statistics with corresponding transcript identifiers.

In some embodiments, a method herein comprises outputting a box plot for transcripts corresponding to unique polypeptides, where the box plot displays transcript expression values in diseased samples for each of a plurality of defined diseases. In some embodiments, a method herein comprises outputting a box plot for transcripts corresponding to unique polypeptides, where the box plot displays transcript expression values in non-diseased samples for each of a plurality of tissues. In some embodiments, a method herein comprises outputting a box plot for transcripts corresponding to partially unique polypeptides, where the box plot displays merged transcript expression values in diseased samples for each of a plurality of defined diseases. In some embodiments, a method herein comprises outputting a box plot for transcripts corresponding to partially unique polypeptides, where the box plot displays merged transcript expression values in non-diseased samples for each of a plurality of tissues.

Certain system structures described herein allow for rapid identification of sub-populations of disease-associated transcripts in a large database. For example, certain precomputed components of a database described herein allow for rapid identification of sub-populations of disease-associated transcripts in a large database. In some embodiments, an output (e.g., a list of transcript identifiers and/or other output features described herein) is generated within 60 seconds. In some embodiments, an output (e.g., a list of transcript identifiers and/or other output features described herein) is generated within 50 seconds. In some embodiments, an output (e.g., a list of transcript identifiers and/or other output features described herein) is generated within 40 seconds. In some embodiments, an output (e.g., a list of transcript identifiers and/or other output features described herein) is generated within 30 seconds. In some embodiments, an output (e.g., a list of transcript identifiers and/or other output features described herein) is generated within 20 seconds. In some embodiments, an output (e.g., a list of transcript identifiers and/or other output features described herein) is generated within 10 seconds. In some embodiments, an output (e.g., a list of transcript identifiers and/or other output features described herein) is generated within 5 seconds. In some embodiments, an output (e.g., a list of transcript identifiers and/or other output features described herein) is generated within 2 seconds.

### Protein Analysis Tool

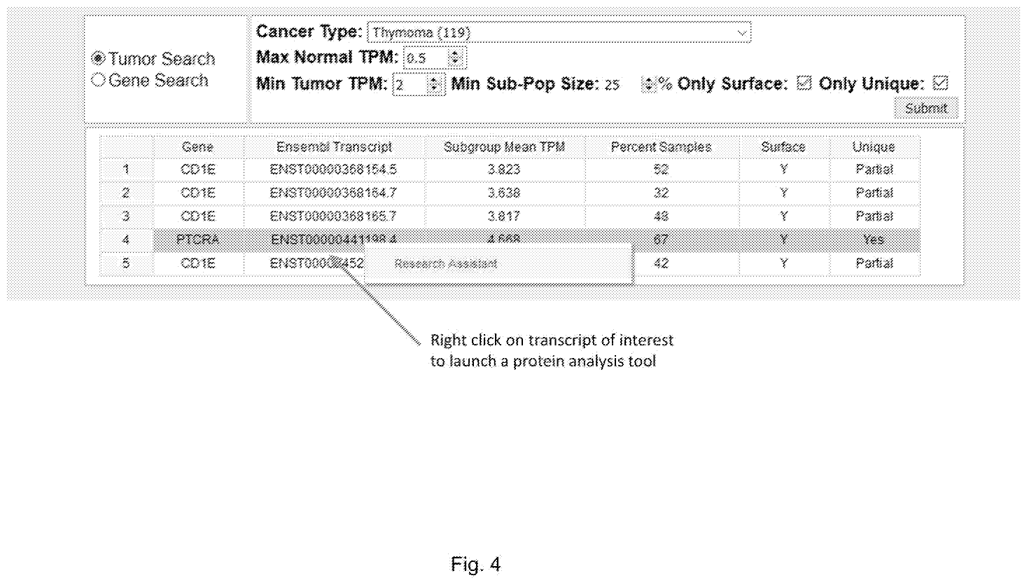

In some embodiments, a method herein includes use of a protein analysis tool. In some embodiments, a system herein includes a protein analysis tool. A protein analysis tool may comprise one or more components useful for analyzing a protein or peptide sequence encoded by a transcript identified by a method or system described herein (see e.g., FIGS. 4-8). For example, a protein analysis tool may allow for rapid identification of structural elements for each transcript identified that can be targeted (e.g., by a binding molecule, for development of a therapy or diagnostic).

In some embodiments, a method herein comprises analyzing a polypeptide. A polypeptide analysis may comprise identifying one or more transcript identifiers in a database (e.g., according to a method described herein), and selecting a transcript identifier (e.g., from a list of outputted transcript identifiers as described herein). For a selected transcript identifier, a polypeptide analysis tool may output one or more features of a polypeptide or peptide encoded by the transcript corresponding to the selected transcript identifier. For example, a polypeptide analysis tool may output a three-dimensional structure corresponding to a polypeptide linear sequence corresponding to the selected transcript identifier. A polypeptide analysis tool may output functional polypeptide domain information for a polypeptide linear sequence corresponding to the selected transcript identifier. In some embodiments, one or more tables of a database described herein relate three-dimensional structure coordinates and functional polypeptide domain information to a polypeptide linear sequence.

In some embodiments, for a selected transcript identifier, a transcript identifier is output for each of one or more transcript isoforms encoded by the gene that encodes the transcript corresponding to the selected transcript identifier. In some embodiments, for a selected transcript identifier, an alignment of polypeptide linear sequences is outputted. In some embodiments, one or more tables of a database described herein relate polypeptide linear sequences to corresponding transcript identifiers. In some embodiments, an outputted three-dimensional structure corresponds to one or more polypeptide linear sequences. In some embodiments, outputted functional polypeptide domain information is for one or more polypeptide linear sequences. In some embodiments, a three-dimensional structure comprises one or more of the following features: the three-dimensional structure is a user-moveable structure, the three dimensional structure is annotated with functional polypeptide domain information, and linear polypeptide sequence(s) is/are mapped to the three-dimensional structure.

In some embodiments, a method herein comprises receiving a defined portion of a linear polypeptide sequence, and displaying a portion of a corresponding three-dimensional structure corresponding to the defined portion of the linear polypeptide sequence. A corresponding three-dimensional structure is a structure to which a linear polypeptide sequence has been mapped. In some embodiments, a method herein comprises receiving a defined portion of a three-dimensional structure, and displaying a portion of a corresponding linear polypeptide sequence corresponding to the defined portion of the three-dimensional structure. In some embodiments, a method herein comprises receiving a defined portion of a three-dimensional structure, and displaying annotated functional polypeptide domain information corresponding to the defined portion of the three-dimensional structure.

In some embodiments, a method herein comprises prior to identifying one or more transcript identifiers, receiving user input comprising one or more of: (i) a defined disease selected from a plurality of defined diseases, where each of the defined diseases corresponds to diseased tissue samples, (ii) a defined minimum transcript expression value threshold for diseased tissue samples, (iii) a defined maximum or a defined median transcript expression value threshold for non-diseased tissues, where each of the non-diseased tissues corresponds to non-diseased tissue samples, and (iv) a defined minimum sample sub-population percentage. In some embodiments, one or more identified transcript identifiers meet one or more of the following criteria (1) and (2): (1) a corresponding transcript expression value in diseased tissue samples corresponding to the input defined disease of (i) is greater than the input defined minimum transcript expression value threshold of (ii), and (2) a corresponding transcript expression value in non-diseased tissue is less than the input defined maximum or defined median transcript expression value threshold of (iii). In some embodiments, one or more identified transcript identifiers meet criteria (1) and/or (2) for a percentage of diseased samples corresponding to an input defined disease greater than a defined minimum sub-population percentage. In some embodiments, a method comprises prior to selecting a transcript identifier, outputting a list comprising a transcript identifier for each of one or more transcripts identified by a method herein, thereby identifying a transcript of one or more genes that is expressed at a level in a defined subpopulation of diseased tissue samples higher than a level the transcript is expressed in non-diseased tissue samples.

In some embodiments, a method herein comprises prior to identifying one or more transcript identifiers, receiving user input comprising one or more of: (i) a defined disease selected from a plurality of defined diseases, where each of the defined diseases corresponds to diseased tissue samples, (ii) a defined transcript expression ratio threshold, where the transcript expression ratio is a ratio of a transcript expression value for diseased tissue samples to a transcript expression value for non-diseased tissues, where each of the non-diseased tissues corresponds to non-diseased tissue samples, and (iii) a defined minimum sample sub-population percentage. In some embodiments, one or more identified transcript identifiers having a corresponding transcript expression ratio for an input defined disease that is greater than an input defined transcript expression ratio threshold. In some embodiments, one or more identified transcript identifiers are identified for a percentage of diseased samples corresponding to an input defined disease greater than a defined minimum sub-population percentage. In some embodiments, a method comprises prior to selecting a transcript identifier, outputting a list comprising a transcript identifier for each of one or more transcripts identified by a method herein, thereby identifying a transcript of one or more genes that is expressed at a level in a defined subpopulation of diseased tissue samples higher than a level the transcript is expressed in non-diseased tissue samples.

### Systems, Machines and Apparatuses

Provided herein are systems, machines and apparatuses for performing a method described herein. Systems, machines and apparatuses may comprise one or more microprocessors.

Systems, machines and apparatuses may comprise memory. Memory may comprise a database described herein. Memory may comprise a database comprising one more tables described herein.

Memory may comprise a database comprising one more tables that relate one or more features described herein. For example, memory may comprise a database comprising one more tables that relate (i) transcript identifiers to genes, where at least a portion of the transcript identifiers correspond to transcript isoform sets, and each of the transcript isoform sets is encoded by a gene; (ii) transcript identifiers to corresponding transcript expression values in diseased tissue samples, and (iii) transcript identifiers to corresponding transcript expression values in non-diseased tissue samples.

Systems, machines and apparatuses may comprise instructions executable by the one or more microprocessors configured to perform a method described herein. For example, systems, machines and apparatuses may comprise instructions executable by the one or more microprocessors configured to perform the following method: (a) receiving user input comprising: (i) a defined disease selected from a plurality of defined diseases, where each of the defined diseases corresponds to diseased tissue samples, (ii) a defined minimum transcript expression value threshold for diseased tissue samples, (iii) a defined maximum or a defined median transcript expression value threshold for non-diseased tissues, where each of the non-diseased tissues corresponds to non-diseased tissue samples, and (iv) a defined minimum sample sub-population percentage; (b) identifying in the database one or more transcript identifiers meeting the following criteria (1) and (2): (1) a corresponding transcript expression value in diseased tissue samples corresponding to the input defined disease of (a)(i) is greater than the input defined minimum transcript expression value threshold of (a)(ii), and (2) a corresponding transcript expression value in non-diseased tissue is less than the input defined maximum or defined median transcript expression value threshold of (a)(iii), for a percentage of diseased samples corresponding to the input defined disease of (a)(i) greater than the defined minimum sub-population percentage of (a)(iv); and (c) outputting a list comprising a transcript identifier for each of one or more transcripts identified in (b).

In certain configurations, systems, machines and apparatuses may comprise instructions executable by the one or more microprocessors configured to perform the following method: (a) receiving user input comprising: (i) a defined disease selected from a plurality of defined diseases, where each of the defined diseases corresponds to diseased tissue samples, (ii) a defined transcript expression ratio threshold, where the transcript expression ratio is a ratio of a transcript expression value for diseased tissue samples to a transcript expression value for non-diseased tissues, where each of the non-diseased tissues corresponds to non-diseased tissue samples, and (iii) a defined minimum sample sub-population percentage; (b) identifying in the database one or more transcript identifiers having a corresponding transcript expression ratio for the input defined disease of (a)(i) that is greater than the input defined transcript expression ratio threshold of (a)(ii), for a percentage of diseased samples corresponding to the input defined disease of (a)(i) greater than the defined minimum sub-population percentage of (a)(iii); and (c) outputting a list comprising a transcript identifier for each of one or more transcripts identified in (b).

### Disease-Associated Isoform Identification System Implementation

Methods described herein for identifying disease-associated transcript isoforms generally are computer-implemented methods. A disease-associated isoform identification system or one or more portions thereof may be implemented with any type of hardware and/or software, and may be a pre-programmed general purpose computing device. For example, the system may be implemented using a server, a personal computer, a portable computer, a thin client, or any suitable device or devices. The system and/or components thereof may be a single device at a single location, or multiple devices at a single, or multiple, locations that are connected together using any appropriate communication protocols over any communication medium such as electric cable, fiber optic cable, or in a wireless manner.

A disease-associated isoform identification system or one or more portions thereof can include clients and servers. A client and server generally are remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A disease-associated isoform identification system or one or more portions thereof can include a back-end component (e.g., a data server), and/or a middleware component (e.g., an application server), and/or a front-end component. A front-end component can be a client computer having a graphical user interface or a Web browser through which a user can interact with an implementation described herein. Components of a disease-associated isoform identification system can be interconnected by any form or medium of digital data communication, e.g., a communication network. Examples of communication networks include a local area network (“LAN”) and a wide area network (“WAN”), an inter network (e.g., the Internet), and peer-to-peer networks (e.g., ad hoc peer-to-peer networks).

A disease-associated isoform identification system or one or more portions thereof can be implemented in digital electronic circuitry, or in computer software, firmware, or hardware, including the structures disclosed in this specification and their structural equivalents, or in combinations of one or more of them. A disease-associated isoform identification system or one or more portions thereof can be implemented as one or more computer programs, i.e., one or more modules of computer program instructions, encoded on computer storage medium for execution by, or to control the operation of, data processing apparatus. Alternatively or in addition, program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. A computer storage medium can also be, or be included in, one or more separate physical components or media (e.g., multiple CDs, disks, or other storage devices).

Operations performed by a disease-associated isoform identification system or one or more portions thereof can be implemented as operations performed by a “data processing apparatus” on data stored on one or more computer-readable storage devices or received from other sources. A “data processing apparatus” encompasses different types of apparatus, devices, and machines for processing data, non-limiting examples of which include a programmable processor, a computer, a system on a chip, or multiples of, or combinations, of the foregoing. An apparatus can include special purpose logic circuitry, e.g., an FPGA (field programmable gate array) or an ASIC (application specific integrated circuit). An apparatus can also include, in addition to hardware, code that creates an execution environment for a computer program, e.g., code that constitutes processor firmware, a protocol stack, a database management system, an operating system, a cross-platform runtime environment, a virtual machine, or a combination of one or more of them. An apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

Processes and logic flows described for a disease-associated isoform identification system or one or more portions thereof can be performed by one or more programmable processors executing one or more computer programs to perform actions by operating on input data and generating output. Processes and logic flows can be performed by, and apparatus can also be implemented as, special purpose logic circuitry, e.g., an FPGA (field programmable gate array) or an ASIC (application-specific integrated circuit).

Processors suitable for the execution of a computer program include general and special purpose microprocessors, for example, and any one or more processors of any kind of digital computer. Generally, a processor receives instructions and data from a read-only memory or a random access memory or both. Essential elements of a computer are a processor for performing actions in accordance with instructions and one or more memory devices for storing instructions and data. A computer can also include, or be operatively coupled to receive data from or transfer data to (or both), one or more mass storage devices for storing data, e.g., magnetic, magneto-optical disks, or optical disks. A computer need not include devices. A computer can be embedded in a device including but not limited to a mobile telephone, a personal digital assistant (PDA), a mobile audio or video player, a Global Positioning System (GPS) receiver, or a portable storage device (e.g., a universal serial bus (USB) flash drive). Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. A processor and memory can be supplemented by, or incorporated in, special purpose logic circuitry.

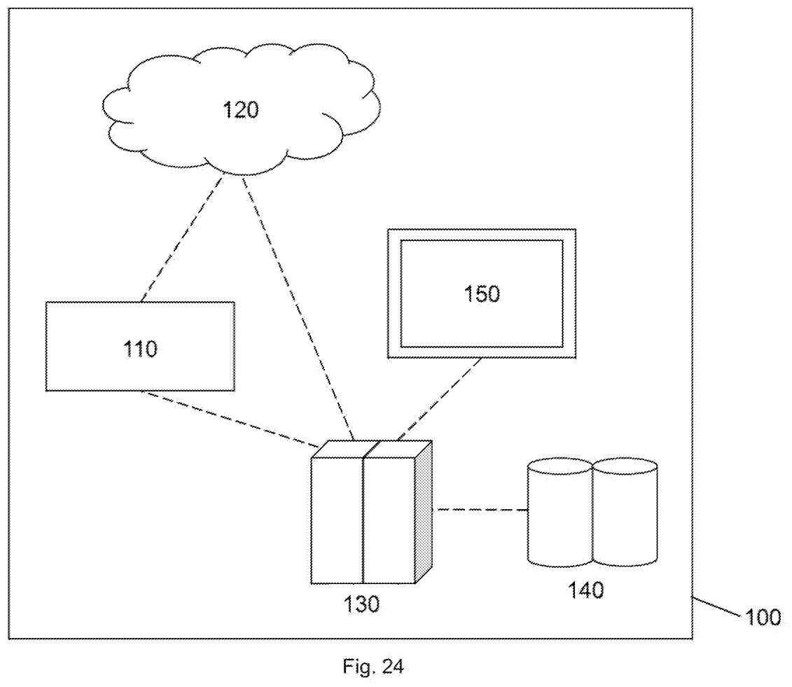

A non-limiting disease-associated isoform identification system implementation is illustrated in FIG. 24. In FIG. 24, illustrated is a disease-associated isoform identification system implementation (100) that includes an interface (110), a network (120), one or more servers (130), memory (140) and a display (150). In certain instances, network (120) can be within one or more servers (130). Certain applications can be co-localized on a single server, and certain applications can be located on two or more separate servers. In certain implementations, memory (140) includes one or more databases. For example, amino acid sequences and associated expression levels can be stored in a database in memory (140). Output for one or more applications described herein can depicted on display (150).

### Use of Amino Acid Subsequences and Amino Acid Sequences Identified by a Method or System Herein

Certain outputs of the methods and systems described herein (e.g., amino acid subsequences, amino acid sequences) may be utilized in a variety of applications. For example, certain applications may include an immunotherapeutic strategy targeting tumor-associated isoforms. In one embodiment, the immunotherapeutic strategy targeting tumor-associated isoforms is a peptide vaccine. In another embodiment, the peptide is encoded by a DNA vector. In another embodiment, the immunotherapeutic strategy targeting tumor-associated isoforms is an adoptive T-cells therapy. In another embodiment, the immunotherapeutic strategy targeting tumor-associated isoforms is monoclonal antibody therapy. In one embodiment, the immunotherapeutic strategy targeting tumor-specific mutations is a peptide vaccine. In another embodiment, the peptide is encoded by a DNA vector. In another embodiment, the immunotherapeutic strategy targeting tumor-specific mutations is an adoptive T-cells therapy. In another embodiment, the immunotherapeutic strategy targeting tumor-specific mutations is monoclonal antibody therapy.

Amino acid subsequences identified by a method or system described herein can be considered immunogenic peptide candidates, which can be utilized in a variety of applications. In certain instances, an immunogenic peptide identified by a method or system described herein can be synthesized. A peptide can be synthesized using any suitable method, including by chemical synthesis, by in vitro translation, or by recombinant translation in host cells. Thus, provided herein is a composition comprising a peptide identified by a method or system described herein and a method for synthesizing a peptide identified by a method or system described herein.

A synthesized peptide can be combined with one or more suitable pharmaceutically acceptable adjuvants and/or one or more suitable pharmaceutically acceptable carriers suitable for a vaccine. Non-limiting examples of pharmaceutically acceptable vaccine adjuvants include aluminum (e.g., amorphous aluminum hydroxyphosphate sulfate (AAHS), aluminum hydroxide, aluminum phosphate, potassium aluminum sulfate); monophosphoryl lipid A (MPL) and aluminum salt (ASO4); oil in water emulsion composed of squalene (MF59); monophosphoryl lipid A (MPL) and QS-21, a natural compound extracted from the Chilean soapbark tree, combined in a liposomal formulation (ASO1B); and cytosine phosphoguanine (CpG 1018). A pharmaceutically acceptable carrier can be a diluent, excipient, or vehicle included in a composition containing the peptide that is administered. A pharmaceutically acceptable carrier can be a sterile liquid. A pharmaceutically acceptable aqueous carrier, such as a saline solution, aqueous dextrose solution and/or glycerol solution can be included when a vaccine is administered intravenously. A vaccine composition that includes an immunogenic peptide identified by a method or system described herein and a pharmaceutically acceptable adjuvant, and optionally includes a pharmaceutically acceptable carrier, can be administered to a subject in need thereof (e.g., human subject) in an amount sufficient to induce an immune response to the peptide in the subject. Such a composition can be administered to a subject as part of a method for treating a condition (e.g., a disease, a cancer) in which inducing an immune response against the peptide can treat the condition (e.g., ameliorate a symptom associated with the condition).

A polynucleotide encoding an immunogenic peptide identified by a method or system described herein can be prepared. A polynucleotide can include one or more elements from a different type of organism from which the polynucleotide portion encoding the immunogenic peptide originated. In certain instances, a polynucleotide can include a polynucleotide portion form a human gene that encodes a peptide identified by a method or system described herein, and can include one or more polynucleotide portions from a different organism (e.g., from a virus; from a bacterium). A polynucleotide sometimes is an expression vector or expression plasmid. A polynucleotide sometimes is a vector or plasmid suitable for administration to a subject, and can be formulated as a vaccine. A polynucleotide vector sometimes is a DNA vector (e.g., a DNA virus or based on a DNA virus (e.g., double-stranded DNA virus), including a herpesvirus, an adenovirus, and a poxvirus) or a RNA vector (e.g., RNA virus or based on a RNA virus, including a retrovirus and a ssRNA virus). Non-limiting examples of polynucleotide vectors are described in Deng et al., Vaccine 33(48): 6938-6946 (2015). A vaccine composition that includes a polynucleotide encoding an immunogenic peptide identified by a method or system described herein, and optionally includes a pharmaceutically acceptable carrier and/or adjuvant, can be administered to a subject in need thereof (e.g., human subject) in an amount sufficient to induce an immune response to the peptide in the subject. Such a composition can be administered to a subject as part of a method for treating a condition (e.g., a disease, a cancer) in which inducing an immune response against the peptide can treat the condition (e.g., ameliorate a symptom associated with the condition).

A composition that includes an antigen presenting cell (APC) and a peptide identified by a method or system described herein can be prepared. In certain implementations, a composition that includes an APC and a polynucleotide encoding a peptide identified by a method or system described herein can be prepared. A composition that includes an APC transduced with a polynucleotide encoding a peptide identified by a method or system described herein can be prepared. A polynucleotide encoding a peptide identified by a method or system described herein sometimes is an expression plasmid or expression vector, and an APC can be transduced by the polynucleotide. An APC can be transduced by a polynucleotide in any suitable manner, non-limiting examples of which include transduction by naked polynucleotide and transduction by electroporation. A non-limiting example of an APC is a dendritic cell. In certain implementations, a composition comprising an APC and a peptide identified by a method or system described herein (e.g., a vaccine composition), or a composition comprising an APC transduced with a polynucleotide encoding a peptide identified by a method or system described herein (e.g., a vaccine composition), where the composition optionally includes a pharmaceutically acceptable carrier and/or adjuvant, can be administered to a subject in need thereof (e.g., human subject) in an amount sufficient to induce an immune response to the peptide in the subject. Such a composition can be administered to a subject as part of a method for treating a condition (e.g., a disease, a cancer) in which inducing an immune response against the peptide can treat the condition (e.g., ameliorate a symptom associated with the condition).

A peptide identified by a method or system described herein can be administered to a subject for production of antibodies that immunospecifically bind to the peptide. Antibodies produced can be polyclonal antibodies or monoclonal antibodies, for example. A peptide identified by a method or system described herein can be included in a composition administered to an animal subject (e.g., rabbit subject, camelid subject), antiserum can be obtained, and polyclonal antibodies optionally may be enriched and/or isolated from the antiserum. A peptide identified by a method or system described herein can be included in a composition administered to an animal subject (e.g., murine subject, guinea pig subject, rabbit subject) and spleen cells from the subject can be combined with myeloma cells under conditions that produce monoclonal antibody generating hybridomas. Hybridomas can be screened for those that produce monoclonal antibodies that immunospecifically bind to the peptide administered to the animal subject. Accordingly, compositions containing the peptide can be administered to a subject as part of a method for manufacturing antibodies (e.g., monoclonal antibodies, polyclonal antibodies) that immunospecifically bind to the peptide.

### Samples

Provided herein are methods for analyzing nucleic acid and/or polypeptides from a sample. Nucleic acid and/or polypeptides may be isolated from a sample obtained from a subject (e.g., a test subject). A subject can be any living or non-living organism, including but not limited to a human, a non-human animal, a plant, a bacterium, a fungus, a protest, or a pathogen. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female. A subject may be any age (e.g., an embryo, a fetus, an infant, a child, an adult). A subject may be a patient having a disease or condition, a patient suspected of having a disease or condition, a patient in remission for a disease or condition, a patient with a family history of a disease or condition, and/or a subject obtaining a screen for a disease or condition. A subject may be a cancer patient, a patient suspected of having cancer, a patient in remission, a patient with a family history of cancer, and/or a subject obtaining a cancer screen. A subject may be a patient having an infection or infectious disease or infected with a pathogen (e.g., bacteria, virus, fungus, protozoa, and the like), a patient suspected of having an infection or infectious disease or being infected with a pathogen, a patient recovering from an infection, infectious disease, or pathogenic infection, a patient with a history of infections, infectious disease, pathogenic infections, and/or a subject obtaining an infectious disease or pathogen screen.

A sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism (e.g., bacteria, yeast, or the like).

A sample or test sample may be any specimen that is isolated or obtained from a subject or part thereof (e.g., a human subject, a subject having a disease or condition, a cancer patient, a patient having an infection or infectious disease, a tumor, an infected organ or tissue, a diseased organ or tissue). Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., cancer biopsy), celocentesis sample, cells (blood cells, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof.

In some embodiments, a sample is isolated or obtained from a specimen (e.g., healthy or diseased specimen), cell(s) (e.g., healthy or diseased cell), tissue (e.g., healthy or diseased tissue), organ (e.g., healthy or diseased organ), and/or the like of an animal (e.g., an animal subject). A sample obtained from a healthy specimen, cell, tissue, and/or organ may be referred to as a non-disease or non-diseased sample. A non-disease or non-diseased sample may be obtained from a subject with no diagnosis of a particular disease (e.g., cancer), no history of a particular disease (e.g., cancer), and/or no suspicion of having a particular disease (e.g., cancer). A sample obtained from a diseased specimen, cell, tissue, and/or organ may be referred to as a disease sample or diseased sample. A disease or diseased sample may be obtained from a subject with a diagnosis of a particular disease or condition (e.g., cancer), a history of a particular disease or condition (e.g., cancer), and/or a subject suspected of having a particular disease or condition (e.g., cancer).

A sample can be a liquid sample. Examples of liquid samples include, but are not limited to, blood or a blood product (e.g., serum, plasma, or the like), urine, cerebral spinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy).

A sample may be a tumor sample (i.e., a sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation. Examples of cancer include, but are not limited to, acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, chronic myeloid leukemia, acute lymphocytic leukemia, multiple myeloma, non-Hodgkin lymphoma, Hodgkin lymphoma, marginal zone lymphoma, follicular lymphoma, small lymphocyte lymphoma, B-cell lymphoma, diffuse large B-cell lymphoma or mantle cell lymphoma, acute lymphoblastic leukemia, adrenocortical cancer, cervical and endocervical cancer, clear cell sarcoma of the kidney, kidney clear cell carcinoma, kidney papillary cell carcinoma, neuroblastoma, testicular germ cell tumor, uterine corpus endometrioid carcinoma, Wilms tumor, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like.

A sample may be a non-diseased sample (i.e., a sample isolated from a non-diseased tissue). Examples of non-diseased tissue sources include, but are not limited to, adipose—subcutaneous, adipose—visceral (omentum), adrenal gland, artery—aorta, artery—coronary, artery—tibial, brain—amygdala, brain—anterior cingulate cortex (BA24), brain—caudate (basal ganglia), brain —cerebellar hemisphere, brain—cerebellum, brain—cortex, brain—frontal cortex (BA9), brain —hippocampus, brain—hypothalamus, brain—nucleus accumbens (basal ganglia), brain—putamen (basal ganglia), brain—spinal cord (cervical c-1), brain—substantia nigra, breast—mammary tissue, colon—sigmoid, colon—transverse, esophagus—gastroesophageal junction, esophagus —mucosa, esophagus—muscularis, heart—atrial appendage, heart—left ventricle, kidney—cortex, kidney—medulla, liver, lung, minor salivary gland, muscle—skeletal, nerve—tibial, ovary, pancreas, pituitary, prostate, skin—not sun exposed (suprapubic), skin—sun exposed (lower leg), small intestine—terminal ileum, spleen, stomach, testis, thyroid, uterus, vagina, whole blood, bladder, cervix—endocervix, cervix—ectocervix, fallopian tube, cells—EBV-transformed lymphocytes, and cells—cultured fibroblasts.

### Nucleic Acid

Nucleic acid may be analyzed using the methods and systems described herein. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), polynucleotide sequence(s), and the like may be used interchangeably throughout the disclosure. The terms refer to nucleic acids of any composition, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), short inhibitory RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed in a tumor, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), alternative splice variants, and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

Nucleic acid analyzed by the methods and systems described herein may be from a category or collection of nucleic acids. For example, nucleic acid may from a genome, a transcriptome, a library (e.g., a DNA library (a genomic DNA library, a cDNA library), an RNA library (an mRNA library)), a nucleic acid pool, and the like or combinations thereof. A genome generally refers to a complete list of nucleotides (A, C, G, and T) that make up the chromosomes of an individual or a species, and includes both the genes (coding regions) and noncoding DNA, and may include mitochondrial DNA. A transcriptome generally refers to a set of RNA transcripts, including coding and non-coding, in an individual or a population of cells, and sometimes refers to all RNAs, or just mRNA, depending on the context. Data obtained from a transcriptome may be used to analyze processes such as cellular differentiation, carcinogenesis, transcription regulation, and biomarker discovery, for example. The transcriptome is related to other “omes” such as, for example, the proteome, metabolome, translatome, exome, meiome, and thanatotranscriptome, which describe specific types of RNA transcripts. A nucleic acid library generally refers to a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

### Polypeptides

Polypeptides may be analyzed using the methods and systems described herein. A polypeptide generally refers to a polymer, linked by peptide bonds, that has a sequence of amino acids encoded by a polynucleotide. A polypeptide may refer to a linear sequence of amino acids and/or may refer to a three dimensional protein. Proteins or portions thereof (e.g., a subunit of a protein) are generally made up of polypeptides. A peptide generally refers to a portion or fragment of a larger polypeptide. In some instances, a peptide refers to a polymer containing between about 2 amino acids to about 10 amino acids, 2 amino acids to about 20 amino acids, or about 2 amino acids to about 30 amino acids. Peptides, may include, for example, dipeptides, tripeptides, tetrapeptides, and oligopeptides. Amino acids that have been incorporated into peptides and/or polypeptides may be referred to as residues. Peptides and polypeptides typically have an N-terminal (amine group) residue at one end and C-terminal (carboxyl group) residue at the opposite end, and amino acid sequences are typically read in the N-terminal to C-terminal direction.

### Certain Implementations

Following are non-limiting examples of certain implementations of the technology.
A1. A computer-implemented method for identifying a transcript of a gene that is expressed at a level in a defined subpopulation of diseased tissue samples higher than a level the transcript is expressed in non-diseased tissue samples, comprising:
(a) receiving user input comprising:
   (i) a defined disease selected from a plurality of defined diseases, wherein each of the defined diseases corresponds to diseased tissue samples,
   (ii) a defined minimum transcript expression value threshold for diseased tissue samples,
   (iii) a defined maximum or defined median transcript expression value threshold for non-diseased tissues, wherein each of the non-diseased tissues corresponds to non-diseased tissue samples, and
   (iv) a defined minimum sample sub-population percentage;
(b) identifying in a database comprising one or more tables that relate:
   (i) transcript identifiers to genes, wherein:
   at least a portion of the transcript identifiers correspond to transcript isoform sets, and each of the transcript isoform sets is encoded by a gene;
   (ii) transcript identifiers to corresponding transcript expression values in diseased tissue samples, and
   (iii) transcript identifiers to corresponding transcript expression values in non-diseased tissue samples;
   one or more transcript identifiers meeting the following criteria (1) and (2):
      (1) a corresponding transcript expression value in diseased tissue samples corresponding to the input defined disease of (a)(i) is greater than the input defined minimum transcript expression value threshold of (a)(ii), and
      (2) a corresponding transcript expression value in non-diseased tissue is less than the input defined maximum or defined median transcript expression value threshold of (a)(iii),
   for a percentage of diseased samples corresponding to the input defined disease of (a)(i) greater than the defined minimum sub-population percentage of (a)(iv); and
(c) outputting a list comprising a transcript identifier for each of one or more transcripts identified in (b), thereby identifying a transcript of one or more genes that is expressed at a level in a defined subpopulation of diseased tissue samples higher than a level the transcript is expressed in non-diseased tissue samples.
A2. The method of embodiment A1, wherein the defined minimum sample sub-population percentage of (a)(iv) is selected from a plurality of defined minimum sample sub-population percentages.
A2.1. The method of embodiment A1 or A2, wherein the defined minimum transcript expression value threshold for diseased tissue samples is selected from a plurality of defined minimum transcript expression value thresholds for diseased tissue samples.
A2.2. The method of any one of embodiments A1-A2.1, wherein the defined maximum transcript expression value threshold for non-diseased tissues is selected from a plurality of defined maximum transcript expression value thresholds for non-diseased tissues.
A2.3. The method of any one of embodiments A1-A2.1, wherein the defined median transcript expression value threshold for non-diseased tissues is selected from a plurality of defined median transcript expression value thresholds for non-diseased tissues.
A3. The method of any one of embodiments A1-A2.3, wherein:
   in (b), the one or more tables in the database relate percentages of diseased tissue samples to corresponding transcript identifiers for each defined disease, and
   each of the percentages is calculated based on an amount of diseased tissue samples for which the transcript expression values of a corresponding transcript identifier exceed the defined minimum transcript expression value threshold of (a)(ii).
A3.1. The method of embodiment A3, wherein each of the percentages is calculated for each defined minimum transcript expression value threshold of the plurality of defined minimum transcript expression value thresholds of (a)(ii).
A4. The method of embodiment A3 or A3.1, wherein each of the percentages equals (i) the number of diseased samples for a defined disease for which transcript expression values for a corresponding transcript identifier exceeds the defined minimum transcript expression value threshold, divided by (ii) total number of diseased samples for the defined disease.
A5. The method of any one of embodiments A3-A4, wherein the list outputted in (c) comprises the percentage of samples exceeding defined minimum transcript expression value threshold of (a)(ii) for each of the transcript identifiers listed.
A6. The method of any one of embodiments A1-A5, wherein:
   in (b), the one or more tables in the database relate average transcript expression values to corresponding transcript identifiers for each defined disease, and
   each of the average transcript expression values is calculated based on transcript expression values for diseased tissue samples for which the transcript expression values exceed the defined minimum transcript expression value threshold of (a)(ii).
A6.1. The method of embodiment A6, wherein each of the average transcript expression values is calculated for each defined minimum transcript expression value threshold of the plurality of defined minimum transcript expression value thresholds of (a)(ii).
A7. The method of embodiment A6 or A6.1, wherein the list outputted in (c) comprises the average transcript expression value for each of the transcript identifiers listed.
A8. The method of embodiment A6 or A7, wherein the average transcript expression value is a median transcript expression value.
A9. The method of any one of embodiments A1-A8, comprising:
(d) receiving, after (c), user selection of a transcript identifier outputted in (c).
A10. The method of embodiment A9, comprising:
(e) outputting the transcript identifier selected in (d) with a transcript identifier for each of one or more transcript isoforms encoded by the gene that encodes the transcript corresponding to the transcript identifier selected in (d).
A11. The method of embodiment A10, comprising, for each transcript identifier outputted, outputting an average transcript expression value in diseased tissue samples corresponding to the input defined disease of (a)(i), and one or more of:
   (i) an average transcript expression value for all non-diseased samples or a subset of non-diseased samples,
   (ii) a maximum transcript expression value of non-diseased samples, and
   (iii) a non-diseased tissue corresponding to a maximum transcript expression value;
wherein the one or more tables of the database relate the average transcript expression values in diseased tissue samples, the average transcript expression values for all non-diseased samples or a subset of non-diseased samples, the maximum transcript expression values of non-diseased samples, and the non-diseased tissues corresponding to the maximum transcript expression values, to corresponding transcript identifiers.
A11.1. The method of embodiment A10, comprising, for each transcript identifier outputted, outputting an average transcript expression value in diseased tissue samples corresponding to the input defined disease of (a)(i), and one or more of:
   (i) an average transcript expression value for all non-diseased samples or a subset of non-diseased samples,
   (ii) a median transcript expression value of non-diseased samples, and
   (iii) a non-diseased tissue corresponding to a median transcript expression value;
wherein the one or more tables of the database relate the average transcript expression values in diseased tissue samples, the average transcript expression values for all non-diseased samples or a subset of non-diseased samples, the median transcript expression values of non-diseased samples, and the non-diseased tissues corresponding to the median transcript expression values, to corresponding transcript identifiers.
A12. The method of embodiment A11 or A11.1, wherein the average transcript expression value is a mean transcript expression value.
A12.1 The method of embodiment A11 or A11.1, wherein the average transcript expression value is a median transcript expression value.
A13. The method of any one of embodiments A10-A12.1, comprising outputting an alignment of polypeptide linear sequences corresponding to the transcript identifiers outputted in (e), wherein the one or more tables of the database relate polypeptide linear sequences to corresponding transcript identifiers.
A14. The method of any one of embodiments A9-A13, comprising:
outputting a box plot for the transcript corresponding to the transcript identifier selected in (d), wherein:
   the box plot displays transcript expression values corresponding to the transcript identifier selected in (d) in diseased samples for each of the plurality of defined diseases of (a)(i), and the one or more tables of the database relate transcript expression values and box plot statistics with corresponding transcript identifiers.
A15. The method of any one of embodiments A9-A14, comprising:
outputting a box plot for the transcript corresponding to the transcript identifier selected in (d), wherein:
   the box plot displays transcript expression values corresponding to the transcript identifier selected in (d) in non-diseased samples for each of a plurality of tissues, and
   the one or more tables of the database relate transcript expression values and box plot statistics with corresponding transcript identifiers.
A16. The method of embodiment A14 or A15, wherein the box plot statistics comprise one or more of: average transcript expression value, first quantile of average transcript expression value, third quantile of average transcript expression value, maximum transcript expression value whisker, minimum transcript expression value whisker, and outlier designations for transcripts of samples in each tissue.
A17. The method of embodiment A16, wherein the average transcript expression value is a mean transcript expression value.
A17.1 The method of embodiment A16, wherein the average transcript expression value is a median transcript expression value.
A18. The method of any one of embodiments A13-A17.1, comprising outputting one or more of:
   a three-dimensional structure corresponding to one or more of the polypeptide linear sequences, and
   functional polypeptide domain information, wherein the one or more tables of the database relate three-dimensional structure coordinates and functional polypeptide domain information to the polypeptide linear sequences.
A19. The method of embodiment A18, wherein the three-dimensional structure comprises one or more of the following features:
   the three-dimensional structure is a user-moveable structure,
   the three dimensional structure is annotated with the functional polypeptide domain information, and
   the linear polypeptide sequences are mapped to the three-dimensional structure.
A20. The method of embodiment A19, comprising:
   receiving a defined portion of a linear polypeptide sequence, and
   displaying a portion of a corresponding three-dimensional structure corresponding to the defined portion of the linear polypeptide sequence.
A21. The method of embodiment A19, comprising:
   receiving a defined portion of a three-dimensional structure, and
   displaying a portion of a corresponding linear polypeptide sequence corresponding to the defined portion of the three-dimensional structure.
A22. The method of any one of embodiments A19-A21, comprising:
   receiving a defined portion of a three-dimensional structure, and
   displaying annotated functional polypeptide domain information corresponding to the defined portion of the three-dimensional structure.
A23. The method of any one of embodiments A1-A22, wherein the one or more tables of the database comprises one or more of the following tables:
   a samples table comprising one record for each sample and including phenotype information for each sample;
   a tissue table comprising one row for each tissue type and comprising tissues corresponding to the diseased tissue samples and tissues corresponding to the non-diseased tissue samples;
   a transcript table relating gene identifiers to corresponding transcript identifiers;
   a non-diseased sample statistics table relating transcript expression values to corresponding transcript identifiers for non-diseased samples, and comprising one or more of: average transcript expression value, first quantile of average transcript expression value, third quantile of average transcript expression value, maximum transcript expression value whisker, minimum transcript expression value whisker, and outlier designations for transcripts of non-diseased samples, for corresponding transcript identifiers;
   a non-diseased sample statistics by tissue table relating transcript expression values to corresponding transcript identifiers categorized by tissue, and comprising one or more of: average transcript expression value, first quantile of average transcript expression value, third quantile of average transcript expression value, maximum transcript expression value whisker, minimum transcript expression value whisker, outlier designations for transcripts of samples in each tissue, and the tissue having the highest expression value for each transcript identifier, for corresponding transcript identifiers;
   a diseased sample statistics by tissue table relating transcript expression values to transcript identifiers categorized by tissue, and comprising one or more of: average transcript expression value, first quantile of average transcript expression value, third quantile of average transcript expression value, maximum transcript expression value whisker, minimum transcript expression value whisker, outlier designations for transcripts of samples in each tissue;
   a diseased sample percentage table relating percentages of diseased tissue samples for each defined disease to corresponding transcript identifiers, and
   an aligned linear sequences table relating transcript identifiers to corresponding linear polypeptide sequences.
A23.1 The method of embodiment A23, wherein, in the diseased sample percentage table, each of the percentages is calculated (1) based on transcript expression values for diseased tissue samples for which the transcript expression values exceed the defined minimum transcript expression value threshold of (a)(ii), and (2) for each defined minimum sample sub-population percentage.
A23.2 The method of embodiment A23 or A23.1, wherein:
   the diseased sample statistics by tissue table relates average transcript expression values for each defined disease to corresponding transcript identifiers, and
   each of the average transcript expression values is calculated (1) based on transcript expression values for diseased tissue samples for which the transcript expression values exceed the defined minimum transcript expression value threshold of (a)(ii), and (2) for each defined minimum transcript expression value threshold of the plurality of defined minimum transcript expression value thresholds of (a)(ii).
A23.3 The method of embodiment A23.2, wherein the average transcript expression values are mean transcript expression values.
A23.4 The method of embodiment A23.2, wherein the average transcript expression values are median transcript expression values.
A24. The method of any one of embodiments A23-A23.4, wherein:
   the one or more tables comprise the transcript table,
   the transcript table identifies a subset of gene identifiers each corresponding to a gene encoding a cell surface protein,
   the user input received in (a) comprises selection of a filter for outputting transcript identifiers corresponding to genes encoding cell surface proteins, and
   the one or more transcript identifiers identified in (b) correspond to genes encoding cell surface proteins.
A25. The method of any one of embodiments A23-A24, wherein:
   the one or more tables comprise the transcript table,
   the transcript table identifies a subset of transcript identifiers each corresponding to a transcript encoding a unique polypeptide comprising an insertion, deletion or substitution of one or more amino acids relative to polypeptides encoded by other transcript isoforms of the same gene;
   the user input received in (a) comprises selection of a filter for outputting transcript identifiers corresponding to transcripts encoding unique polypeptides, and
   the one or more transcript identifiers identified in (b) correspond to transcripts encoding unique polypeptides.
A25.1 The method of any one of embodiments A23-A24, wherein:
   the one or more tables comprise the transcript table,
   the transcript table identifies a subset of transcript identifiers each corresponding to a transcript encoding a unique and/or partially unique polypeptide comprising an insertion, deletion or substitution of one or more amino acids relative to polypeptides encoded by other transcript isoforms of the same gene;
   the user input received in (a) comprises selection of a filter for outputting transcript identifiers corresponding to transcripts encoding unique and/or partially unique polypeptides, and
   the one or more transcript identifiers identified in (b) correspond to transcripts encoding unique and/or partially unique polypeptides.
A25.2 The method of embodiment A25.1, wherein the user input further comprises selection of a function for merging the expression values for the transcripts encoding the partially unique polypeptides, thereby generating merged transcript expression values.
A25.3. The method of embodiment A25.2, comprising:
outputting a box plot for the transcripts corresponding to the partially unique polypeptides, wherein:
   the box plot displays merged transcript expression values in diseased samples for each of the plurality of defined diseases of (a)(i).
A25.4. The method of embodiment A25.2 or A25.3, comprising:
outputting a box plot for the transcripts corresponding to the partially unique polypeptides, wherein:
   the box plot displays merged transcript expression values in non-diseased samples for each of a plurality of tissues.
A26. The method of any one of embodiments A1-A25.4, wherein:
   the one or more tables comprise the transcript table,
   the transcript table identifies a subset of transcript identifiers each as a primary transcript having an expression value higher than the expression values corresponding to other transcript isoforms encoded by the same gene, and
   when outputted, transcript identifiers identified as a primary transcript are ranked higher relative to transcript identifiers corresponding to other transcript isoforms encoded by the same gene.
A27. The method of any one of embodiments A1-A26, wherein the transcript expression values in the one or more tables have been processed according to a common gene model.
A28. The method of embodiment A27, wherein the common gene model is a single Gencode version.
A29. The method of embodiment A27 or A28, comprising entering additional transcript expression level values for diseased tissue samples, wherein the expression level values have been processed according to the same gene model, without entering an expression level value for a comparative non-diseased sample.
A30. The method of any one of embodiments A1-A29, wherein the transcript expression values are transcripts per million (TPM) values.
A31. The method of embodiment A30, wherein the TPM values have been generated by a RNA-Seq by Expectation-Maximization (RSEM) algorithm.
A32. The method of any one of embodiments A1-A31, wherein in (a) all of the defined diseases of the plurality of defined diseases of (a)(i) are received as input.
A32.1. The method of any one of embodiments A1-A31, wherein in (a) a subset of the defined diseases of the plurality of defined diseases of (a)(i) is received as input.
A33. The method of any one of embodiments A1-A32.1, wherein the one or more tables comprise transcript expression values for non-diseased tissue samples that were from multiple subjects different than the subjects from which diseased tissue samples were obtained.
A34. The method of embodiment A33, wherein the one or more tables comprise:
   transcript expression values for diseased tissue samples from a TCGA database, and
   transcript expression values for non-diseased tissue samples from a GTEx database.
A35. The method of embodiment A33 or A34, wherein the one or more tables comprise transcript expression values for non-diseased tissue adjacent to diseased tissue in the same subjects from the TCGA database.
A35.1 The method of embodiment A33 or A34, wherein the one or more tables exclude transcript expression values for non-diseased tissue adjacent to diseased tissue in the same subjects from the TCGA database.
A36. The method of any one of embodiments A1-A35, wherein the defined diseases comprise defined cancers.
A37. The method of embodiment A36, wherein the one or more tables comprise transcript expression values from diseased tissue from child subjects.
A38. The method of embodiment A36 or A37, comprising defined cancers chosen from acute lymphoblastic leukemia, acute myeloid leukemia, adrenocortical cancer, bladder urothelial carcinoma, brain lower grade glioma, breast invasive carcinoma, cervical & endocervical cancer, cholangiocarcinoma, clear cell sarcoma of the kidney, colon adenocarcinoma, diffuse large B-cell lymphoma, esophageal carcinoma, glioblastoma multiforme, head & neck squamous cell carcinoma, kidney chromophobe, kidney clear cell carcinoma, kidney papillary cell carcinoma, liver hepatocellular carcinoma, lung adenocarcinoma, lung squamous cell carcinoma, mesothelioma, neuroblastoma, ovarian serous cystadenocarcinoma, pancreatic adenocarcinoma, pheochromocytoma & paraganglioma, prostate adenocarcinoma, rectum adenocarcinoma, sarcoma, skin cutaneous melanoma, stomach adenocarcinoma, testicular germ cell tumor, thymoma, thyroid carcinoma, uterine carcinosarcoma, uterine corpus endometrioid carcinoma, uveal melanoma, and Wilms tumor.
A39. The method of any one of embodiments A1-A38, wherein the non-diseased tissues comprise tissues chosen from adipose—subcutaneous, adipose—visceral (omentum), adrenal gland, artery—aorta, artery—coronary, artery—tibial, brain—amygdala, brain—anterior cingulate cortex (BA24), brain—caudate (basal ganglia), brain—cerebellar hemisphere, brain—cerebellum, brain—cortex, brain—frontal cortex (BA9), brain—hippocampus, brain—hypothalamus, brain—nucleus accumbens (basal ganglia), brain—putamen (basal ganglia), brain—spinal cord (cervical c-1), brain—substantia nigra, breast—mammary tissue, colon—sigmoid, colon—transverse, esophagus —gastroesophageal junction, esophagus—mucosa, esophagus—muscularis, heart—atrial appendage, heart—left ventricle, kidney—cortex, kidney—medulla, liver, lung, minor salivary gland, muscle—skeletal, nerve—tibial, ovary, pancreas, pituitary, prostate, skin—not sun exposed (suprapubic), skin—sun exposed (lower leg), small intestine—terminal ileum, spleen, stomach, testis, thyroid, uterus, vagina, whole blood, bladder, cervix—endocervix, cervix—ectocervix, fallopian tube, cells—EBV-transformed lymphocytes, and cells—cultured fibroblasts.
A40. The method of any one of embodiments A1-A39, wherein the one or more tables comprise transcript expression values for at least 50,000 transcripts for at least 19,000 genes, at least 10,000 diseased samples, at least 7,000 non-diseased samples, at least 35 defined diseases and 45 non-diseased tissues.
A41. The method of any one of embodiments A1-A40, wherein the output of (c) is generated within 60 seconds.
A42. The method of any one of embodiments A1-A41, wherein the user input in (a) further comprises selecting a subset of the non-diseased tissues.
A43. The method of embodiment A42, wherein the defined maximum or defined median transcript expression value threshold for non-diseased tissues in (a)(iii) is determined according to the subset of the non-diseased tissues.
A44. The method of embodiment A42 or A43, wherein (b) comprises identifying in the database one or more transcript identifiers meeting the following criteria (1) and (2):
   (1) a corresponding transcript expression value in diseased tissue samples corresponding to the input defined disease of (a)(i) is greater than the input defined minimum transcript expression value threshold of (a)(ii), and
   (2) a corresponding transcript expression value in the subset of non-diseased tissues is less than the input defined maximum or defined median transcript expression value threshold of (a)(iii).
B1. A computer-implemented method for identifying a transcript of a gene that is expressed at a level in a defined subpopulation of diseased tissue samples higher than a level the transcript is expressed in non-diseased tissue samples, comprising:
(a) receiving user input comprising:
   (i) a defined disease selected from a plurality of defined diseases, wherein each of the defined diseases corresponds to diseased tissue samples,
   (ii) a defined transcript expression ratio threshold, wherein the transcript expression ratio is a ratio of a transcript expression value for diseased tissue samples to a transcript expression value for non-diseased tissues, wherein each of the non-diseased tissues corresponds to non-diseased tissue samples, and
   (iii) a defined minimum sample sub-population percentage;
(b) identifying in a database comprising one or more tables that relate:
   (i) transcript identifiers to genes, wherein:
   at least a portion of the transcript identifiers correspond to transcript isoform sets, and each of the transcript isoform sets is encoded by a gene; and
   (ii) transcript identifiers to corresponding transcript expression ratios;
   one or more transcript identifiers having a corresponding transcript expression ratio for the input defined disease of (a)(i) that is greater than the input defined transcript expression ratio threshold of (a)(ii),
   for a percentage of diseased samples corresponding to the input defined disease of (a)(i) greater than the defined minimum sub-population percentage of (a)(iii); and
(c) outputting a list comprising a transcript identifier for each of one or more transcripts identified in (b), thereby identifying a transcript of one or more genes that is expressed at a level in a defined subpopulation of diseased tissue samples higher than a level the transcript is expressed in non-diseased tissue samples.
B1.1 The method of embodiment B1, wherein the transcript expression value for non-diseased tissues in (a)(ii) corresponds to the highest transcript expression value of all non-diseased tissues.
B1.2 The method of embodiment B1, wherein the transcript expression value for non-diseased tissues in (a)(ii) corresponds to the highest transcript expression value of a selected subset of non-diseased tissues.
B2. The method of any one of embodiments B1-B1.2, wherein the defined minimum sample sub-population percentage of (a)(iii) is selected from a plurality of defined minimum sample sub-population percentages.
B2.1. The method of any one of embodiments B1-B2, wherein the defined transcript expression ratio threshold is selected from a plurality of defined transcript expression ratio thresholds.
B3. The method of any one of embodiments B1-E2.1, wherein:
   in (b), the one or more tables in the database relate percentages of diseased tissue samples to corresponding transcript identifiers for each defined disease, and
   each of the percentages is calculated based on an amount of diseased tissue samples for which the transcript expression ratios of a corresponding transcript identifier exceed the defined transcript expression ratio threshold of (a)(ii).
B3.1. The method of embodiment B3, wherein each of the percentages is calculated for each defined transcript expression ratio threshold of the plurality of defined transcript expression ratio thresholds.
B4. The method of embodiment B3 or B3.1, wherein each of the percentages equals (i) the number of diseased samples for a defined disease for which transcript expression ratios for a corresponding transcript identifier exceeds the defined transcript expression ratio threshold, divided by (ii) total number of diseased samples for the defined disease.
B5. The method of any one of embodiments B3-B4, wherein the list outputted in (c) comprises the percentage of samples exceeding defined transcript expression ratio threshold of (a)(ii) for each of the transcript identifiers listed.
B6. The method of any one of embodiments B1-B5, comprising:
(d) receiving, after (c), user selection of a transcript identifier outputted in (c).
B7. The method of embodiment B6, comprising:
(e) outputting the transcript identifier selected in (d) with a transcript identifier for each of one or more transcript isoforms encoded by the gene that encodes the transcript corresponding to the transcript identifier selected in (d).
B8. The method of embodiment B7, comprising, for each transcript identifier outputted, outputting an average transcript expression value in diseased tissue samples corresponding to the input defined disease of (a)(i), and one or more of:
   (i) an average transcript expression value for all non-diseased samples or a subset of non-diseased samples,
   (ii) a maximum transcript expression value of non-diseased samples, and
   (iii) a non-diseased tissue corresponding to a maximum transcript expression value;
wherein the one or more tables of the database relate the average transcript expression values in diseased tissue samples, the average transcript expression values for all non-diseased samples or a subset of non-diseased samples, the maximum transcript expression values of non-diseased samples, and the non-diseased tissues corresponding to the maximum transcript expression values, to corresponding transcript identifiers.
B9. The method of embodiment B7, comprising, for each transcript identifier outputted, outputting an average transcript expression value in diseased tissue samples corresponding to the input defined disease of (a)(i), and one or more of:
   (i) an average transcript expression value for all non-diseased samples or a subset of non-diseased samples,
   (ii) a median transcript expression value of non-diseased samples, and
   (iii) a non-diseased tissue corresponding to a median transcript expression value;
wherein the one or more tables of the database relate the average transcript expression values in diseased tissue samples, the average transcript expression values for all non-diseased samples or a subset of non-diseased samples, the median transcript expression values of non-diseased samples, and the non-diseased tissues corresponding to the median transcript expression values, to corresponding transcript identifiers.
B10. The method of embodiment B8 or B9, wherein the average transcript expression value is a mean transcript expression value.
B11. The method of embodiment B8 or B9, wherein the average transcript expression value is a median transcript expression value.
B12. The method of any one of embodiments B7-B11, comprising outputting an alignment of polypeptide linear sequences corresponding to the transcript identifiers outputted in (e), wherein the one or more tables of the database relate polypeptide linear sequences to corresponding transcript identifiers.
B13. The method of any one of embodiments B6-B12, comprising:
outputting a box plot for the transcript corresponding to the transcript identifier selected in (d), wherein:
   the box plot displays transcript expression values corresponding to the transcript identifier selected in (d) in diseased samples for each of the plurality of defined diseases of (a)(i), and
   the one or more tables of the database relate transcript expression values and box plot statistics with corresponding transcript identifiers.
B14. The method of any one of embodiments B6-B13, comprising:
outputting a box plot for the transcript corresponding to the transcript identifier selected in (d), wherein:
   the box plot displays transcript expression values corresponding to the transcript identifier selected in (d) in non-diseased samples for each of a plurality of tissues, and
   the one or more tables of the database relate transcript expression values and box plot statistics with corresponding transcript identifiers.
B15. The method of embodiment B13 or B14, wherein the box plot statistics comprise one or more of: average transcript expression value, first quantile of average transcript expression value, third quantile of average transcript expression value, maximum transcript expression value whisker, minimum transcript expression value whisker, and outlier designations for transcripts of samples in each tissue.
B16. The method of embodiment B15, wherein the average transcript expression value is a mean transcript expression value.
B17. The method of embodiment B15, wherein the average transcript expression value is a median transcript expression value.
B18. The method of any one of embodiments B12-B17, comprising outputting one or more of:
   a three-dimensional structure corresponding to one or more of the polypeptide linear sequences, and
   functional polypeptide domain information, wherein the one or more tables of the database relate three-dimensional structure coordinates and functional polypeptide domain information to the polypeptide linear sequences.
B19. The method of embodiment B18, wherein the three-dimensional structure comprises one or more of the following features:
   the three-dimensional structure is a user-moveable structure,
   the three dimensional structure is annotated with the functional polypeptide domain information, and
   the linear polypeptide sequences are mapped to the three-dimensional structure.
B20. The method of embodiment B19, comprising:
   receiving a defined portion of a linear polypeptide sequence, and
   displaying a portion of a corresponding three-dimensional structure corresponding to the defined portion of the linear polypeptide sequence.
B21. The method of embodiment B19, comprising:
   receiving a defined portion of a three-dimensional structure, and
   displaying a portion of a corresponding linear polypeptide sequence corresponding to the defined portion of the three-dimensional structure.
B22. The method of any one of embodiments B19-B21, comprising:
   receiving a defined portion of a three-dimensional structure, and
   displaying annotated functional polypeptide domain information corresponding to the defined portion of the three-dimensional structure.
B23. The method of any one of embodiments B1-B22, wherein the one or more tables of the database comprises one or more of the following tables:
   a samples table comprising one record for each sample and including phenotype information for each sample;
   a tissue table comprising one row for each tissue type and comprising tissues corresponding to the diseased tissue samples and tissues corresponding to the non-diseased tissue samples;
   a transcript table relating gene identifiers to corresponding transcript identifiers;
   a non-diseased sample statistics table relating transcript expression values to corresponding transcript identifiers for non-diseased samples, and comprising one or more of: average transcript expression value, first quantile of average transcript expression value, third quantile of average transcript expression value, maximum transcript expression value whisker, minimum transcript expression value whisker, and outlier designations for transcripts of non-diseased samples, for corresponding transcript identifiers;
   a non-diseased sample statistics by tissue table relating transcript expression values to corresponding transcript identifiers categorized by tissue, and comprising one or more of: average transcript expression value, first quantile of average transcript expression value, third quantile of average transcript expression value, maximum transcript expression value whisker, minimum transcript expression value whisker, outlier designations for transcripts of samples in each tissue, and the tissue having the highest expression value for each transcript identifier, for corresponding transcript identifiers;
   a diseased sample statistics by tissue table relating transcript expression values to transcript identifiers categorized by tissue, and comprising one or more of: average transcript expression value, first quantile of average transcript expression value, third quantile of average transcript expression value, maximum transcript expression value whisker, minimum transcript expression value whisker, outlier designations for transcripts of samples in each tissue;
   a diseased sample percentage table relating percentages of diseased tissue samples for each defined disease to corresponding transcript identifiers, and
   an aligned linear sequences table relating transcript identifiers to corresponding linear polypeptide sequences.
B24. The method of embodiment B23, wherein:
   the one or more tables comprise the transcript table,
   the transcript table identifies a subset of gene identifiers each corresponding to a gene encoding a cell surface protein,
   the user input received in (a) comprises selection of a filter for outputting transcript identifiers corresponding to genes encoding cell surface proteins, and
   the one or more transcript identifiers identified in (b) correspond to genes encoding cell surface proteins.
B25. The method of embodiment B23 or B24, wherein:
   the one or more tables comprise the transcript table,
   the transcript table identifies a subset of transcript identifiers each corresponding to a transcript encoding a unique polypeptide comprising an insertion, deletion or substitution of one or more amino acids relative to polypeptides encoded by other transcript isoforms of the same gene;
   the user input received in (a) comprises selection of a filter for outputting transcript identifiers corresponding to transcripts encoding unique polypeptides, and
   the one or more transcript identifiers identified in (b) correspond to transcripts encoding unique polypeptides.
B25.1 The method of embodiment B23 or B24, wherein:
   the one or more tables comprise the transcript table,
   the transcript table identifies a subset of transcript identifiers each corresponding to a transcript encoding a unique and/or partially unique polypeptide comprising an insertion, deletion or substitution of one or more amino acids relative to polypeptides encoded by other transcript isoforms of the same gene;
   the user input received in (a) comprises selection of a filter for outputting transcript identifiers corresponding to transcripts encoding unique and/or partially unique polypeptides, and
   the one or more transcript identifiers identified in (b) correspond to transcripts encoding unique and/or partially unique polypeptides.
B25.2 The method of embodiment B25.1, wherein the user input further comprises selection of a function for merging the expression values for the transcripts encoding the partially unique polypeptides, thereby generating merged transcript expression values.
B25.3. The method of embodiment B25.2, comprising:
outputting a box plot for the transcripts corresponding to the partially unique polypeptides, wherein:
   the box plot displays merged transcript expression values in diseased samples for each of
   the plurality of defined diseases of (a)(i).
B25.4. The method of embodiment B25.2 or B25.3, comprising:
outputting a box plot for the transcripts corresponding to the partially unique polypeptides, wherein:
   the box plot displays merged transcript expression values in non-diseased samples for each of a plurality of tissues.
B26. The method of any one of embodiments B1-B25.4, wherein:
   the one or more tables comprise the transcript table,
   the transcript table identifies a subset of transcript identifiers each as a primary transcript having an expression value higher than the expression values corresponding to other transcript isoforms encoded by the same gene, and
   when outputted, transcript identifiers identified as a primary transcript are ranked higher relative to transcript identifiers corresponding to other transcript isoforms encoded by the same gene.
B27. The method of any one of embodiments B1-B26, wherein the transcript expression values in the one or more tables have been processed according to a common gene model.
B28. The method of embodiment B27, wherein the common gene model is a single Gencode version.
B29. The method of embodiment B27 or B28, comprising entering additional transcript expression level values for diseased tissue samples, wherein the expression level values have been processed according to the same gene model, without entering an expression level value for a comparative non-diseased sample.
B30. The method of any one of embodiments B1-B29, wherein the transcript expression values are transcripts per million (TPM) values.
B31. The method of embodiment B30, wherein the TPM values have been generated by a RNA-Seq by Expectation-Maximization (RSEM) algorithm.
B32. The method of any one of embodiments B1-B31, wherein in (a) all of the defined diseases of the plurality of defined diseases of (a)(i) are received as input.
B32.1. The method of any one of embodiments B1-B31, wherein in (a) a subset of the defined diseases of the plurality of defined diseases of (a)(i) is received as input.
B33. The method of any one of embodiments B1-B32.1, wherein the one or more tables comprise transcript expression values for non-diseased tissue samples that were from multiple subjects different than the subjects from which diseased tissue samples were obtained.
B34. The method of embodiment B33, wherein the one or more tables comprise:
   transcript expression values for diseased tissue samples from a TCGA database, and
   transcript expression values for non-diseased tissue samples from a GTEx database.
B35. The method of embodiment B33 or B34, wherein the one or more tables comprise transcript expression values for non-diseased tissue adjacent to diseased tissue in the same subjects from the TCGA database.
B35.1 The method of embodiment B33 or B34, wherein the one or more tables exclude transcript expression values for non-diseased tissue adjacent to diseased tissue in the same subjects from the TCGA database.
B36. The method of any one of embodiments B1-B35, wherein the defined diseases comprise defined cancers.
B37. The method of embodiment B36, wherein the one or more tables comprise transcript expression values from diseased tissue from child subjects.
B38. The method of embodiment B36 or B37, comprising defined cancers chosen from acute lymphoblastic leukemia, acute myeloid leukemia, adrenocortical cancer, bladder urothelial carcinoma, brain lower grade glioma, breast invasive carcinoma, cervical & endocervical cancer, cholangiocarcinoma, clear cell sarcoma of the kidney, colon adenocarcinoma, diffuse large B-cell lymphoma, esophageal carcinoma, glioblastoma multiforme, head & neck squamous cell carcinoma, kidney chromophobe, kidney clear cell carcinoma, kidney papillary cell carcinoma, liver hepatocellular carcinoma, lung adenocarcinoma, lung squamous cell carcinoma, mesothelioma, neuroblastoma, ovarian serous cystadenocarcinoma, pancreatic adenocarcinoma, pheochromocytoma & paraganglioma, prostate adenocarcinoma, rectum adenocarcinoma, sarcoma, skin cutaneous melanoma, stomach adenocarcinoma, testicular germ cell tumor, thymoma, thyroid carcinoma, uterine carcinosarcoma, uterine corpus endometrioid carcinoma, uveal melanoma, and Wilms tumor.
B39. The method of any one of embodiments B1-B38, wherein the non-diseased tissues comprise tissues chosen from adipose—subcutaneous, adipose—visceral (omentum), adrenal gland, artery—aorta, artery—coronary, artery—tibial, brain—amygdala, brain—anterior cingulate cortex (BA24), brain—caudate (basal ganglia), brain—cerebellar hemisphere, brain—cerebellum, brain—cortex, brain—frontal cortex (BA9), brain—hippocampus, brain—hypothalamus, brain—nucleus accumbens (basal ganglia), brain—putamen (basal ganglia), brain—spinal cord (cervical c-1), brain—substantia nigra, breast—mammary tissue, colon—sigmoid, colon—transverse, esophagus—gastroesophageal junction, esophagus—mucosa, esophagus—muscularis, heart—atrial appendage, heart—left ventricle, kidney—cortex, kidney—medulla, liver, lung, minor salivary gland, muscle—skeletal, nerve—tibial, ovary, pancreas, pituitary, prostate, skin—not sun exposed (suprapubic), skin—sun exposed (lower leg), small intestine—terminal ileum, spleen, stomach, testis, thyroid, uterus, vagina, whole blood, bladder, cervix—endocervix, cervix—ectocervix, fallopian tube, cells—EBV-transformed lymphocytes, and cells—cultured fibroblasts.
B40. The method of any one of embodiments B1-B39, wherein the one or more tables comprise transcript expression values for at least 50,000 transcripts for at least 19,000 genes, at least 10,000 diseased samples, at least 7,000 non-diseased samples, at least 35 defined diseases and 45 non-diseased tissues.
B41. The method of any one of embodiments B1-B40, wherein the output of (c) is generated within 60 seconds.
B42. The method of any one of embodiments B1-B41, wherein the user input in (a) further comprises selecting a subset of the non-diseased tissues.
C1. A system comprising one or more microprocessors and memory, the memory comprising:
   a database comprising one or more tables that relate:
   (i) transcript identifiers to genes, wherein:
   at least a portion of the transcript identifiers correspond to transcript isoform sets, and each of the transcript isoform sets is encoded by a gene;
   (ii) transcript identifiers to corresponding transcript expression values in diseased tissue samples, and
   (iii) transcript identifiers to corresponding transcript expression values in non-diseased tissue samples; and
   instructions executable by the one or more microprocessors configured to perform the following method:
(a) receiving user input comprising:
   (i) a defined disease selected from a plurality of defined diseases, wherein each of the defined diseases corresponds to diseased tissue samples,
   (ii) a defined minimum transcript expression value threshold for diseased tissue samples,
   (iii) a defined maximum or a defined median transcript expression value threshold for non-diseased tissues, wherein each of the non-diseased tissues corresponds to non-diseased tissue samples, and
   (iv) a defined minimum sample sub-population percentage;
(b) identifying in the database one or more transcript identifiers meeting the following criteria (1) and (2):
   (1) a corresponding transcript expression value in diseased tissue samples corresponding to the input defined disease of (a)(i) is greater than the input defined minimum transcript expression value threshold of (a)(ii), and
   (2) a corresponding transcript expression value in non-diseased tissue is less than the input defined maximum or defined median transcript expression value threshold of (a)(iii),
   for a percentage of diseased samples corresponding to the input defined disease of (a)(i) greater than the defined minimum sub-population percentage of (a)(iv); and
(c) outputting a list comprising a transcript identifier for each of one or more transcripts identified in (b).
C2. The system of embodiment C1, wherein the instructions are configured to perform the method according to any one of embodiments A2-A44.
C3. A system comprising one or more microprocessors and memory, the memory comprising:
   a database comprising one or more tables that relate:
   (i) transcript identifiers to genes, wherein:
   at least a portion of the transcript identifiers correspond to transcript isoform sets, and
   each of the transcript isoform sets is encoded by a gene;
   (ii) transcript identifiers to corresponding transcript expression values in diseased tissue samples, and
   (iii) transcript identifiers to corresponding transcript expression values in non-diseased tissue samples; and
   instructions executable by the one or more microprocessors configured to perform the following method:
(a) receiving user input comprising:
   (i) a defined disease selected from a plurality of defined diseases, wherein each of the defined diseases corresponds to diseased tissue samples,
   (ii) a defined transcript expression ratio threshold, wherein the transcript expression ratio is a ratio of a transcript expression value for diseased tissue samples to a transcript expression value for non-diseased tissues, wherein each of the non-diseased tissues corresponds to non-diseased tissue samples, and
   (iii) a defined minimum sample sub-population percentage;
(b) identifying in the database one or more transcript identifiers having a corresponding transcript expression ratio for the input defined disease of (a)(i) that is greater than the input defined transcript expression ratio threshold of (a)(ii), for a percentage of diseased samples corresponding to the input defined disease of (a)(i) greater than the defined minimum sub-population percentage of (a)(iii); and
(c) outputting a list comprising a transcript identifier for each of one or more transcripts identified in (b).
C4. The system of embodiment C3, wherein the instructions are configured to perform the method according to any one of embodiments B2 to B42.
D1. A computer-implemented method for analyzing a polypeptide comprising:
(a) identifying one or more transcript identifiers in a database comprising one or more tables that relate:
   (i) transcript identifiers to genes, wherein:
   at least a portion of the transcript identifiers correspond to transcript isoform sets, and
   each of the transcript isoform sets is encoded by a gene;
   (ii) transcript identifiers to corresponding transcript expression values in diseased tissue samples, and
   (iii) transcript identifiers to corresponding transcript expression values in non-diseased tissue samples;
(b) receiving user selection of a transcript identifier; and
(c) outputting one or more of:
   (i) a three-dimensional structure corresponding to a polypeptide linear sequence corresponding to the selected transcript identifier, and
   (ii) functional polypeptide domain information for a polypeptide linear sequence corresponding to the selected transcript identifier, wherein the one or more tables of the database relate three-dimensional structure coordinates and functional polypeptide domain information to the polypeptide linear sequence.
D2. The method of embodiment D1, further comprising after (b), outputting the transcript identifier selected in (b) with a transcript identifier for each of one or more transcript isoforms encoded by the gene that encodes the transcript corresponding to the transcript identifier selected in (b).
D3. The method of embodiment D2, further comprising outputting an alignment of polypeptide linear sequences corresponding to the transcript identifiers outputted in (c), wherein the one or more tables of the database relate polypeptide linear sequences to corresponding transcript identifiers.
D4. The method of embodiment D3, wherein the three-dimensional structure outputted in (c)(i) corresponds to one or more of the polypeptide linear sequences.
D5. The method of embodiment D3 or D4, wherein the functional polypeptide domain information outputted in (c)(ii) is for one or more of the polypeptide linear sequences.
D6. The method of any one of embodiments D1-D5, wherein the three-dimensional structure comprises one or more of the following features:
   the three-dimensional structure is a user-moveable structure,
   the three dimensional structure is annotated with the functional polypeptide domain information, and
   the linear polypeptide sequence(s) is/are mapped to the three-dimensional structure.
D7. The method of embodiment D6, comprising:
   receiving a defined portion of a linear polypeptide sequence, and
   displaying a portion of a corresponding three-dimensional structure corresponding to the defined portion of the linear polypeptide sequence.
D8. The method of embodiment D6, comprising:
   receiving a defined portion of a three-dimensional structure, and
   displaying a portion of a corresponding linear polypeptide sequence corresponding to the defined portion of the three-dimensional structure.
D9. The method of any one of embodiments D6-D8, comprising:
   receiving a defined portion of a three-dimensional structure, and
   displaying annotated functional polypeptide domain information corresponding to the defined portion of the three-dimensional structure.
D10. The method of any one of embodiments D1-D9, further comprising prior to (a) receiving user input comprising:
   (i) a defined disease selected from a plurality of defined diseases, wherein each of the defined diseases corresponds to diseased tissue samples,
   (ii) a defined minimum transcript expression value threshold for diseased tissue samples,
   (iii) a defined maximum or a defined median transcript expression value threshold for non-diseased tissues, wherein each of the non-diseased tissues corresponds to non-diseased tissue samples, and
   (iv) a defined minimum sample sub-population percentage.
D11. The method of embodiment D10, wherein the one or more transcript identifiers identified in (a) meet the following criteria (1) and (2):
   (1) a corresponding transcript expression value in diseased tissue samples corresponding to the input defined disease of (i) is greater than the input defined minimum transcript expression value threshold of (ii), and
   (2) a corresponding transcript expression value in non-diseased tissue is less than the input defined maximum or defined median transcript expression value threshold of (iii).
D12. The method of embodiment D10 or D11, wherein one or more transcript identifiers identified in (a) meet criteria (1) and (2) for a percentage of diseased samples corresponding to the input defined disease of (i) greater than the defined minimum sub-population percentage of (iv).
D13. The method of any one of embodiments D1-D9, further comprising prior to (a) receiving user input comprising:
   (i) a defined disease selected from a plurality of defined diseases, wherein each of the defined diseases corresponds to diseased tissue samples,
   (ii) a defined transcript expression ratio threshold, wherein the transcript expression ratio is a ratio of a transcript expression value for diseased tissue samples to a transcript expression value for non-diseased tissues, wherein each of the non-diseased tissues corresponds to non-diseased tissue samples, and
   (iii) a defined minimum sample sub-population percentage.
D14. The method of embodiment D13, wherein the one or more transcript identifiers identified in (a) have a corresponding transcript expression ratio for the input defined disease of (i) that is greater than the input defined transcript expression ratio threshold of (ii).
D15. The method of embodiment D13 or D14, wherein the one or more transcript identifiers identified in (a) have a corresponding transcript expression ratio for the input defined disease of (i) that is greater than the input defined transcript expression ratio threshold of (ii) for a percentage of diseased samples corresponding to the input defined disease of (i) greater than the defined minimum sub-population percentage of (iii).
D16. The method of any one of embodiments D1-D15, further comprising prior (b) to outputting a list comprising a transcript identifier for each of one or more transcripts identified in (b), thereby identifying a transcript of one or more genes that is expressed at a level in a defined subpopulation of diseased tissue samples higher than a level the transcript is expressed in non-diseased tissue samples.
D17. The method of any one of embodiments D1-D16, further comprising any one of the features of any one of embodiments A2-A44 and B2 to B42.
E1. A method for generating a database comprising:
   (i) relating transcript identifiers to genes, wherein:
   at least a portion of the transcript identifiers correspond to transcript isoform sets, and each of the transcript isoform sets is encoded by a gene;
   (ii) relating transcript identifiers to corresponding transcript expression values in diseased tissue samples for a plurality of defined diseases; and
   (iii) relating percentages of diseased tissue samples for each defined disease to corresponding transcript identifiers, wherein the percentages are based on an amount of diseased tissue samples corresponding to a defined disease for which transcript expression values of a corresponding transcript identifier exceed a defined minimum transcript expression value threshold, or
   (iii′) relating percentages of diseased tissue samples for each defined disease to corresponding transcript identifiers, wherein the percentages are based on an amount of diseased tissue samples corresponding to a defined disease for which transcript expression ratios of a corresponding transcript identifier exceed a defined transcript expression ratio threshold.
E1.1. The method of embodiment E1, wherein each of the percentages is calculated for each defined minimum transcript expression value threshold of a plurality of defined minimum transcript expression value thresholds.
E2. The method of embodiment E1 or E1.1, comprising relating transcript identifiers to corresponding transcript expression values in non-diseased tissue samples.
E3. The method of any one of embodiments E1-E2, comprising relating average transcript expression values to corresponding transcript identifiers for each defined disease.
E3.1. The method of embodiment E3, wherein the average transcript expression values are for a subpopulation of diseased tissue samples for a corresponding defined disease for which transcript expression values of a corresponding transcript identifier exceed a defined minimum transcript expression value threshold.
E3.2. The method of embodiment E3 or E3.1, wherein each of the average transcript expression values is calculated for each defined minimum transcript expression value threshold of a plurality of defined minimum transcript expression value thresholds.
E4. The method of any one of embodiments E1-E3.2, comprising relating average transcript expression values in diseased tissue samples, average transcript expression values for all non-diseased samples or a subset of non-diseased samples, maximum transcript expression values of non-diseased samples, and non-diseased tissues corresponding to the maximum transcript expression values, to corresponding transcript identifiers.
E4.1. The method of any one of embodiments E1-E3.2, comprising relating average transcript expression values in diseased tissue samples, average transcript expression values for all non-diseased samples or a subset of non-diseased samples, median transcript expression values of non-diseased samples, and non-diseased tissues corresponding to the median transcript expression values, to corresponding transcript identifiers.
E5. The method of any one of embodiments E1-E4.1, comprising relating polypeptide linear sequences to corresponding transcript identifiers.
E6. The method of embodiment E5, comprising relating three-dimensional structure coordinates and functional polypeptide domain information to the polypeptide linear sequences.
E7. The method of any one of embodiments E1-E6, comprising relating transcript expression value statistics with corresponding transcript identifiers.
E8. The method of any one of embodiments E1-E7, comprising relating transcript expression value statistics to corresponding transcript identifiers for non-diseased samples, wherein the statistics comprise one or more of: average transcript expression value, first quantile of average transcript expression value, third quantile of average transcript expression value, maximum transcript expression value whisker, minimum transcript expression value whisker, and outlier designations for transcripts of non-diseased samples, for corresponding transcript identifiers.
E9. The method of any one of embodiments E1-E8, comprising relating transcript expression value statistics to corresponding transcript identifiers for non-diseased samples categorized by tissue, wherein the statistics comprise one or more of: average transcript expression value, first quantile of average transcript expression value, third quantile of average transcript expression value, maximum transcript expression value whisker, minimum transcript expression value whisker, outlier designations for transcripts of samples in each tissue, and the tissue having the highest expression value for each transcript identifier, for corresponding transcript identifiers.
E10. The method of any one of embodiments E1-E9, comprising relating transcript expression value statistics to transcript identifiers for diseased samples categorized by tissue, wherein the statistics comprise one or more of: average transcript expression value, first quantile of average transcript expression value, third quantile of average transcript expression value, maximum transcript expression value whisker, minimum transcript expression value whisker, outlier designations for transcripts of samples in each tissue.
E11. The method of any one of embodiments E1-E10, comprising any one of the features of any one of embodiments A1-A41.
E12. A database produced by the method of any one of embodiments E1 to E11.
E13. The database of embodiment E12, comprising any one of the features of any one of embodiments A1-A44 and B1 to 42.

### EXAMPLES

The examples set forth below illustrate certain implementations and do not limit the technology.

### Example 1: Disease-Associated Isoform Database Construction

The disease-associated isoform identification system described herein can perform genome-wide searches of thousands of genes in thousands of tumor and normal samples to find transcript isoforms with distinct peptide targets that are specific to cancer. The distinct peptides that are identified may be candidate targets for immunotherapy. One aspect that makes this system possible is the construction of a database. The construction of an example database useful for a disease-associated isoform identification system is described below.

### External Data Sources

Data from data sources described below was used to construct the database described herein.
1. The Cancer Genome Atlas (TCGA) project was a government funded study of a wide range of genomic information from thousands of cancer samples covering 33 different tumor types (see e.g., World Wide Web Uniform Resource Locator cancer.gov/about-nci/organization/ccg/research/structural-genomics/tcga). The Therapeutically Applicable Research to Generate Effective Treatments (TARGET) project produced similar genomic data on childhood tumors (see e.g., World Wide Web Uniform Resource Locator ocg.cancer.gov/programs/target). The Genotype-Tissue Expression (GTEx) project provided a large collection of genomic data on normal tissues from ˜1000 donors (see e.g., World Wide Web Uniform Resource Locator gtexportal.org/home/).

The data for construction of the database described herein is RNASeq data and, in particular, transcript isoform expression data, which is finer grained than gene expression data. When a gene has multiple transcripts (isoforms) produced by variation in the transcript splicing patterns, isoform expression data provides expression values for each isoform of each gene. Expression levels were measured in a normalized vale to support cross transcript and cross sample comparison (i.e., Transcripts Per Million (TPM) using the RSEM algorithm).

Differential analysis of isoform expression from all three sources above (TCGA, TARGET, GTEx) can accurately be performed if the isoform expression data was generated in a consistent manner using a common set of gene models (gene models can be thought of as the dictionary of transcript isoforms for all genes). The reprocessing of the RNASeq data from TCGA, TARGET, and GTEX by the Xena group at UCSC using the TOIL pipeline provided a single transcript expression dataset constructed with a common gene model, Gencode v23 (see e.g., World Wide Web Uniform Resource Locator xenabrowser.net/datapages/?hub=https://toil.xenahubs.net:443). The dataset used for the database described herein is located at World Wide Web Uniform Resource Locator xenabrowser.net/datapages/?cohort=TCGA %20TARGET %20GTEx&removeHub=https %3A %2F % 2Fxena.treehouse.gi.ucsc.edu %3A443. The following RSEM TPM and TCGA TARGET GTEX selected phenotypes files were used: TcgaTargetGtex_rsem_isoform_tpm and TcgaTargetGTEX_phenotype.
2. Gencode v23. The Gencode reference data was imported for information about genes, transcript isoforms, and the sequence of the protein product of each isoform (see e.g., World Wide Web Uniform Resource Locator encodegenes.org/human/release_23.html). Files were downloaded from World Wide Web Uniform Resource Locator ftp.ebi.ac.uk/pub/databases/gencode/Gencode_human/release_23: gencode.v23.basic.annotation.gtf.gz, gencode.v23.pc_translations.fa, and gencode.v23.metadata.SwissProt.
3. A supplemental table was used to identify genes which produce proteins that will localize to the cell surface (table_S3_surfaceome.txt from Bausch-Fluck et al. (2018) The in silico human surfaceome PNAS 115 (46) E10988-E10997).
4. The Matched Annotation from the NCBI and EBI (MANE) primary transcript file, release_0.9 MANE.GRCh38.v0.9.summary.txt, was used to identify the canonical or primary transcript for each gene. (see e.g., World Wide Web Uniform Resource Locator ensembl.info/2018/10/12/our-new-joint-transcript-initiative-the-matched-annotation-from-the-ncbi-and-ebi-mane-project/).

Database Build Process

The following steps were performed to construct a SQLite relational database. SQLite is a relational database management system contained in a C library and embedded into the end program. Accordingly, the database described herein exists as a file on computers where the disease-associated isoform program is installed. The database is queried by Python webserver code using SQL.
1. Download TcgaTargetGtex_rsem_isoform_tpm and TcgaTargetGTEX_phenotype from Xena.
2. Run the disease-associated isoform data pipeline program, ImportTOILSamples.py
   a. Parse the phenotype file to build a mapping of disease/normal, tissue type, and source study for each sample.
   b. Load the sample table with one record for each sample including phenotype information. Exclude cell line samples, samples without phenotype, and samples in cohorts of insufficient size.
   c. Load the tissue table with one row per tissue type (e.g. normal lung, lung adenocarcinoma, etc.)
3. Download gencode.v23.basic.annotation.gtf.gz, gencode.v23.pc_translations.fa, and gencode.v23.metadata.SwissProt from GenCode.
4. Download table_S3_surfaceome.txt from Bausch-Fluck et al. (supra).
5. Download release_0.9 MANE.GRCh38.v0.9.summary.txt from MANE.
6. Run the disease-associated isoform pipeline program, ImportGencode.py
   a. Parse GenCode SwissProt file to load the UniProt ID of each transcript.
   b. Parse the surfaceome file to load a list of genes that produce cell surface proteins.
   c. Load the primary Ensembl transcript id for each gene from the MANE file.
   d. Parse the GenCode GTF file and create a transaction record for each transcript indicating the gene symbol, transcript ID and type, and identification of primary transcript and cell surface proteins. Include only protein coding transcripts.
   e. Parse the GenCode translation file to create an entry in the transcript sequence table for each loaded transcript.
7. Run the disease-associated isoform pipeline program, NormalStatsByTissue, to summarize TPM data for normal samples.
   a. Query the Sample table to load an in-memory dictionary of the tissue type for each normal sample.
   b. Query the Transcript table to load an in-memory dictionary of all valid transcripts and their IDs.
   c. Process the Xena RSEM transcript expression file one transcript at a time. For each valid transcript, collect expression values for all normal (non-cancer) samples. Also collect all normal expression values categorized by tissue type (lung, brain, etc). After all samples for the transcript are loaded, summarize the normal samples and normal samples by tissue with the following statistics. Store the results in the Normal Stats table and Normal Stats By Tissue table:
      i. Median, First Quantile, Third Quantile, Whisker High, Whisker Low, and Outliers. These are used for box plot graphics and target search queries. A boxplot is a standardized way of displaying the distribution of data based on a five number summary (“minimum”, first quartile (Q1), median, third quartile (Q3), and “maximum”). A boxplot can identify outliers and provide their values, can indicate whether data is symmetrical, can show how tightly data is grouped, and can show if and how data is skewed.
      ii. Identify and store the normal tissue that had the highest expression for the transcript.
8. Run the disease-associated isoform pipeline program, TumorStatsByTissue
   a. Query the Sample table to load an in-memory dictionary of the tissue type for each tumor sample.
   b. Query the Transcript table to load an in-memory dictionary of all valid transcripts and their IDs.
   c. Process the Xena RSEM transcript expression file one transcript at a time. For each valid transcript, collect expression values for all tumor samples. Also collect all tumor expression values categorized by disease type (lung, adeno, etc.). After all samples for the transcript are loaded, summarize the tumor samples and tumor samples by tissue with the following statistics. Store the results in the Tumor Stats By Tissue table and the Tumor Pct By TPM table. The ‘All’ tumor type stats are used for cross tumor type searches later. Also, the precomputed stats for all possible TPM thresholds are useful for timely exploration of the data for targets by researchers.
      i. Median, First Quantile, Third Quantile, Whisker High, Whisker Low, and Outliers.
      ii. For TPM threshold expression values of 1 through 10, compute the % of samples exceeding the threshold for each tumor type and the median expression of samples exceeding the threshold.
9. Run the disease-associated isoform pipeline program, differences.py to identify unique peptide targets on transcript variants. This is generally difficult to do and is useful for efficient target searching as time is not wasted on isoforms that do not have unique peptides that could be therapy targets.
   a. Retrieve a list of genes, related transcript isoforms, and protein sequence of each isoform from the database.
   b. For each gene with multiple transcript isoforms, perform a multi-sequence alignment of the protein sequences of all the gene's transcripts. Alignment performed with MUSCLE (World Wide Web Uniform Resource Locator ebi.ac.uk/Tools/msa/muscle/).
   c. For each non-primary isoform, walk the alignment looking for sections of protein sequence that differ from all other isoforms. Also look for deleted protein sections which create a unique sequence junction that could be targeted by immunotherapy. Finally, if unique sections are not found, look for sections that differ from the reference transcript. Mark transcripts which have a unique section in the transcript table.
   d. Write the aligned sequence to the Align_Seq table. Mark unique protein sections with special characters.

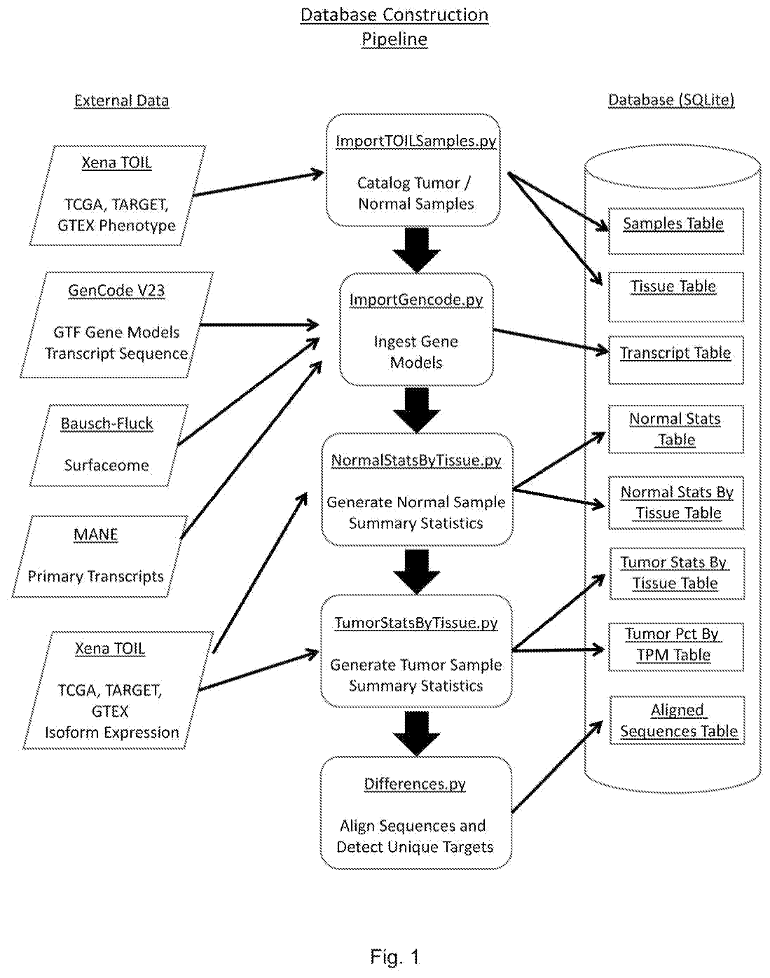

See FIG. 1—Chart of database build process.

### Database Content

The disease-associated isoform database described herein includes expression data for 54,000+ transcripts from 19,500+ genes. There are 10,500+ tumor samples and 7,400+ normal samples. These samples are from 38 different cancer types and include 47 normal tissue types. This is an extremely large dataset with 567+ million transcript expression values.

Cancer types in the database include: acute lymphoblastic leukemia, breast cancer, acute myeloid leukemia, adrenocortical cancer, bile duct cancer, bladder cancer, cervical cancer, colon and rectal cancer, endometrioid cancer, esophageal cancer, glioblastoma, head and neck cancer, kidney chromophobe, kidney clear cell carcinoma, kidney papillary cell carcinoma, large B-cell lymphoma, liver cancer, lower grade glioma, lower grade glioma and glioblastoma, lung adenocarcinoma, lung cancer, lung squamous cell carcinoma, melanoma, mesothelioma, ocular melanomas, ovarian cancer, pancreatic cancer, prostate cancer, sarcoma, stomach cancer, testicular cancer, thymoma, thyroid cancer, and uterine carcinosarcoma. Specific cancer types in the database include: acute lymphoblastic leukemia, acute myeloid leukemia, adrenocortical cancer, bladder urothelial carcinoma, brain lower grade glioma, breast invasive carcinoma, cervical & endocervical cancer, cholangiocarcinoma, clear cell sarcoma of the kidney, colon adenocarcinoma, diffuse large B-cell lymphoma, esophageal carcinoma, glioblastoma multiforme, head & neck squamous cell carcinoma, kidney chromophobe, kidney clear cell carcinoma, kidney papillary cell carcinoma, liver hepatocellular carcinoma, lung adenocarcinoma, lung squamous cell carcinoma, mesothelioma, neuroblastoma, ovarian serous cystadenocarcinoma, pancreatic adenocarcinoma, pheochromocytoma & paraganglioma, prostate adenocarcinoma, rectum adenocarcinoma, sarcoma, skin cutaneous melanoma, stomach adenocarcinoma, testicular germ cell tumor, thymoma, thyroid carcinoma, uterine carcinosarcoma, uterine corpus endometrioid carcinoma, uveal melanoma, and Wilms tumor.

Normal tissue types in the database include: adipose—subcutaneous, adipose—visceral (omentum), adrenal gland, artery—aorta, artery—coronary, artery—tibial, brain—amygdala, brain—anterior cingulate cortex (BA24), brain—caudate (basal ganglia), brain—cerebellar hemisphere, brain—cerebellum, brain—cortex, brain—frontal cortex (BA9), brain—hippocampus, brain—hypothalamus, brain—nucleus accumbens (basal ganglia), brain—putamen (basal ganglia), brain —spinal cord (cervical c-1), brain—substantia nigra, breast—mammary tissue, colon—sigmoid, colon—transverse, esophagus—gastroesophageal junction, esophagus—mucosa, esophagus —muscularis, heart—atrial appendage, heart—left ventricle, kidney—cortex, kidney—medulla, liver, lung, minor salivary gland, muscle—skeletal, nerve—tibial, ovary, pancreas, pituitary, prostate, skin—not sun exposed (suprapubic), skin—sun exposed (lower leg), small intestine—terminal ileum, spleen, stomach, testis, thyroid, uterus, vagina, whole blood, bladder, cervix—endocervix, cervix—ectocervix, fallopian tube, cells—EBV-transformed lymphocytes, and cells—cultured fibroblasts.

The disease-associated isoform program allows researchers to identify and investigate transcript isoforms that are strongly expressed in a specific tumor type or subset of a given tumor type and not expressed at high levels in any normal tissue. Further, the program can specifically identify isoforms in surface proteins with unique, targetable protein segments. This functionality would not be possible without some or all of the database build processes described above.

### Example 2: Disease-Associated Isoform Application

The disease-associated isoform database build process described in Example 1 is generally run once and the completed database can be distributed with the application when it is installed on a computer. The disease-associated isoform application is a dynamic, exploratory piece of software that allows researchers to explore the large volume of TCGA and Target cancer data to find cancer specific peptides (e.g., that are candidates for immunotherapy targets). The disease-associated isoform application is implemented in Python, installed locally on a researcher's workstation, and functions as a local webserver that queries the SQLite database. Researchers use their local web browser to interact with the application. Rank-ordered numerical results are combined with visualizations to present high volume, complex data in an intuitive, interactive fashion that supports rapid analysis and discovery.

### Usage Example

The disease-associated isoform application searches thousands of transcript isoforms in thousands of individuals (samples) to find isoforms that are strongly expressed in tumor cells but not expressed in normal tissue. Rather than comparing tumor to matched normal (e.g. lung adenocarcinoma to normal lung) the disease-associated isoform application described herein compares the tumor samples in the tissue of choice to ALL normal samples in the large GTEX collection. This allows identification of tumor specific isoforms that are not expressed in any of the profiled normal tissues (e.g., to identify targets that would not have toxic off-target effects).

Further, the disease-associated isoform application described herein can identify sub-populations of patients that have strong expression of an isoform that is not expressed in normal tissues. If the selected tumor type occurs in large numbers, then a therapy for a subpopulation could be viable. The disease-associated isoform application also can filter out isoforms that do not produce surface proteins or those that do not have unique, targetable peptides. These filters can save a significant amount of investigator time.

FIG. 2A and FIG. 2B show an example of submitting a search request to the disease-associated isoform application. An HTML interface runs Javascript to send user specified parameters for tumor type, percentage of samples, and expression thresholds to Python code run by a local webserver. The Python code performs a relational SQL query of the database using the supplied parameters. The database build process has previously precomputed for each known protein coding transcript: the box plot upper whisker TPM value in normal tissues (max normal expression), the % of samples exceeding predefined expression levels in tumor tissue, and the average TPM expression in tumor for each of these predefined TPM expression levels. Because of this precompute, it is a simple SQL query to retrieve all transcripts which DO NOT have expression in normal tissue above the user threshold but which DO have expression in tumor above a threshold for at least the specified number of samples. The Python code returns the list of transcripts meeting the user criterion along with the % of samples and average expression of subpopulation above threshold for each.

### Results List

Query results are returned in just a few seconds on an ordinary laptop computer (e.g., Mac laptop with 515 GB SSD, 16 GB DDR4 RAM, 8-core CPU running @3.2 GHz, 8-core GPU, and queries may be run on a computer with lower specifications). The database described herein generally takes up about 1 Gig of hard drive space. An average laptop is typically capable run the software, and a high-end computer typically is not needed. Without the precomputed database design described above, the type of queries used in the disease-associated isoform application generally would not be possible. Database size, memory needed, and response time of a query would be untenable. For example, using just 10% of the data (e.g., about 50 Gig with no precomputed database structure), it took about 20 minutes to run using most of the resources available on a high end computer. Queries against the full data set (i.e., with no precomputed database structure) would take hours or not finish at all. With the precomputed, binned thresholds (e.g., TPM of 1, 2, 3, . . . 10), the queries ran in less than 2 seconds. Accordingly, the precomputed database structure provides compact data and faster performance, and allows a user to query the database using an ordinary computer.

FIGS. 3A-3C show an example of disease-associated isoform application query results. FIG. 3A (top panel) provides a list of all isoforms that meet the search criteria in the selected tumor type. The search criteria are stringent and the number of cancer specific isoforms is small so the resulting list typically is a handful of isoforms. The list provides summary information including the size of the subpopulation meeting the search criteria and average expression level in tumor cells. When investigators select a transcript of interest, the result screen is populated with information and graphics for that isoform (FIG. 3A, bottom two panels; FIG. 3B; FIG. 3C).

Gene Isoforms

The middle panel of FIG. 3A shows all known protein coding isoforms of a selected gene and provides expression levels for each isoform in tumor (“Tumor”) and normal tissue (“All Norm”) including an indication of the normal tissue type (“Max Tissue”) with the highest expression of each isoform (“Max Norm”). This panel provides context so the researcher can understand the range of isoforms of the selected target gene and the expression profile of the isoforms in the selected tumor tissue as well as in normal tissues.

Multi Sequence Alignment

The bottom panel of FIG. 3A shows the protein product of all isoforms of a selected gene, which may be useful for a therapy design process, for example. The top protein sequence (displayed to the user in red font) is the canonical isoform. The highlighted protein sequence is the selected tumor specific isoform. This panel allows investigators to scan up/down to see which sections of the protein product are common to all isoforms and which sections differ. The portion of the tumor specific isoform indicated by the arrow (displayed to the user in yellow font) identifies the unique section that may serve as the seed for a candidate immunotherapy target. Deleted sections in the selected isoform can also create unique protein sections that can be targeted. The way that the tool shows all the protein sequences and computationally identifies unique targets can increase the productivity of investigators designing immunotherapies.

Normal Expression Box Plot

FIG. 3B shows an example box plot for expression of a selected isoform for thousands of normal samples categorized by tissue type. The box plot shows mean expression, high value, low value, 25^{th} percentile, and 75^{th} percentile. It also shows outliers as black circles. A good immunotherapy target isoform generally should not have strong expression in normal tissue. Strong expression in normal tissue may indicate the possibility of toxic off-target therapy impacts. The box plot quickly and effectively shows the landscape of isoform expression in normal tissue. In the example shown in FIG. 3A, there is very little expression in normal tissues at all. There are some outliers and some expression in whole blood but all expression falls below 0.1 TPM.

Tumor Expression Box Plot

FIG. 3C shows an example box plot for expression of a selected isoform for thousands of tumor samples categorized by tissue/disease type. The box plot shows mean expression, high value, low value, 25^{th} percentile, and 75^{th} percentile. In this example, the box plot shows strong expression of the selected isoform in Thymoma—the selected cancer type of the query. This panel also shows non-query tumor types where the therapy may be effective. Other tumor types with strong expression or subpopulations with expression of the selected variant may also respond to a candidate immunotherapy.

### Example 3: Protein Analysis Tool

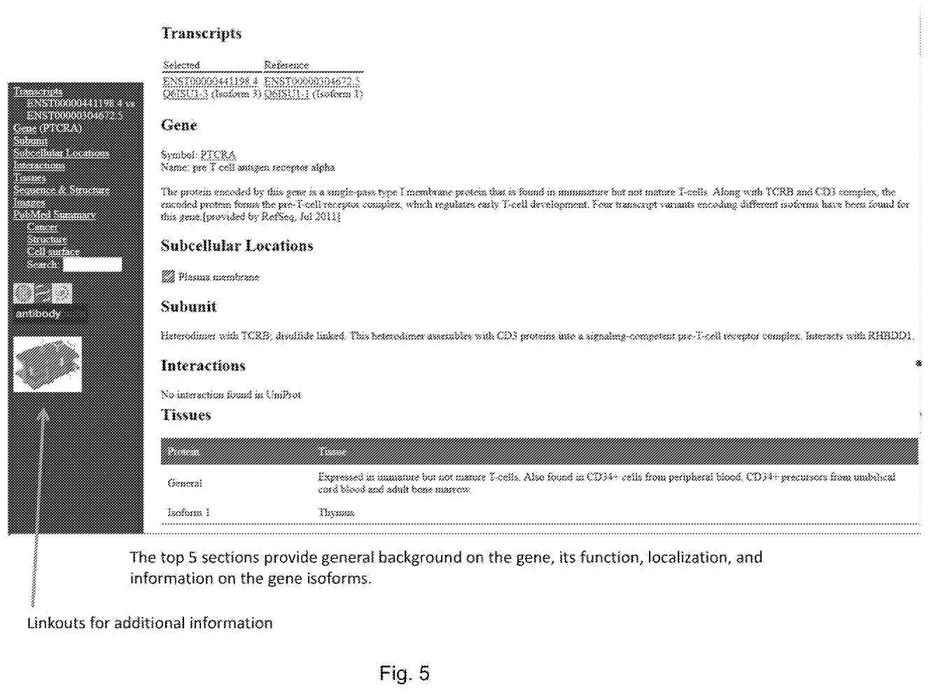

The disease-associated isoform identification system described herein may include a protein analysis tool as a companion application. A protein analysis tool can provide “deep dive” information on a gene and/or transcript isoform. When a potential immunotherapy target is identified using the disease-associated isoform application described herein, and more details are desired, a protein analysis tool may be launched. The protein analysis tool was designed to integrate and automate multiple manually intensive tasks and to establish certain background checks for each candidate target. In the disease-associated isoform application results screen, a user can right click on a selected transcript to launch a protein analysis tool (see e.g., FIG. 4). The protein analysis tool gathers information dynamically from several web-based sources and displays the results in an easy-to-navigate, integrated interface (see e.g., FIG. 5).

### Gene Information

The first several sections in the example protein analysis tool described herein provide background information on the gene and linkouts to sources for additional information. Information known about isoforms is presented (see e.g., FIG. 5).

### Sequence and Structure

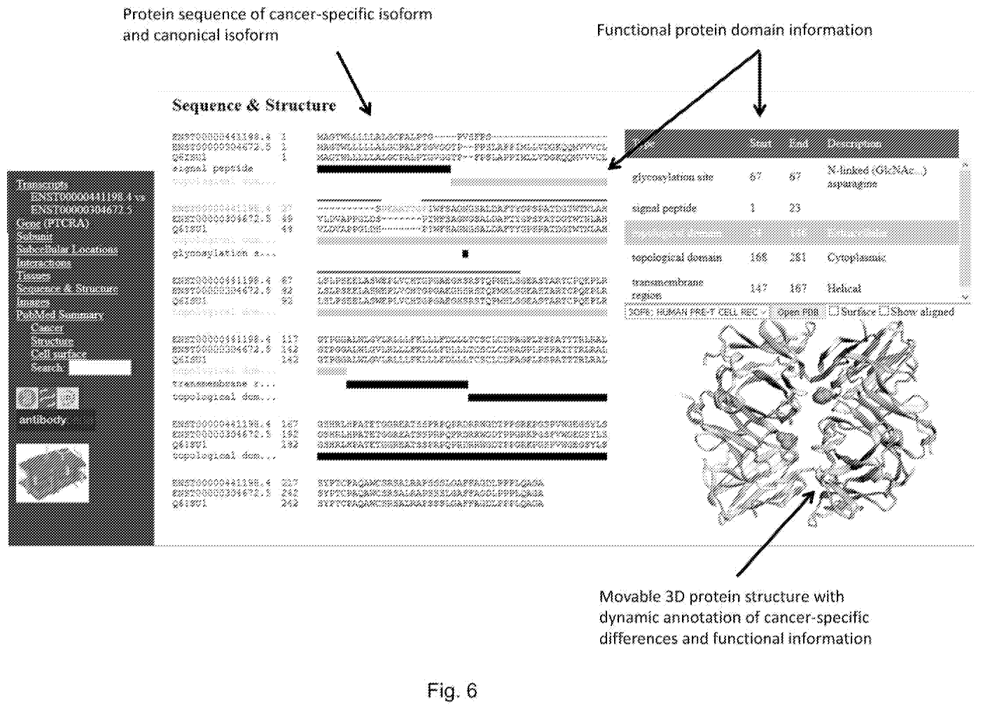

FIG. 6 shows a sequence and structure component of the protein analysis tool described herein. This is a dynamic panel that combines a protein sequence of the canonical form with the cancer specific form. This component of the protein analysis tool blends in information about known functional domains of the protein and, when possible, shows a 3D protein structure with annotation. All of the elements are integrated. A user can set the color of a functional region and that color is reflected in the sequence alignment, functional elements table, and 3D structure. The elements are also dynamic in that selecting an amino acid in the protein sequence shows its position in the protein structure. One advantage of this component is the ability to identify where cancer specific peptide sections reside on the protein, whether the peptide will be accessible to immunotherapies, and how the peptide relates to functional elements of the protein or ligands in the structures, thus reducing or eliminating the need for manual investigation and/or unproductive efforts on useless targets.

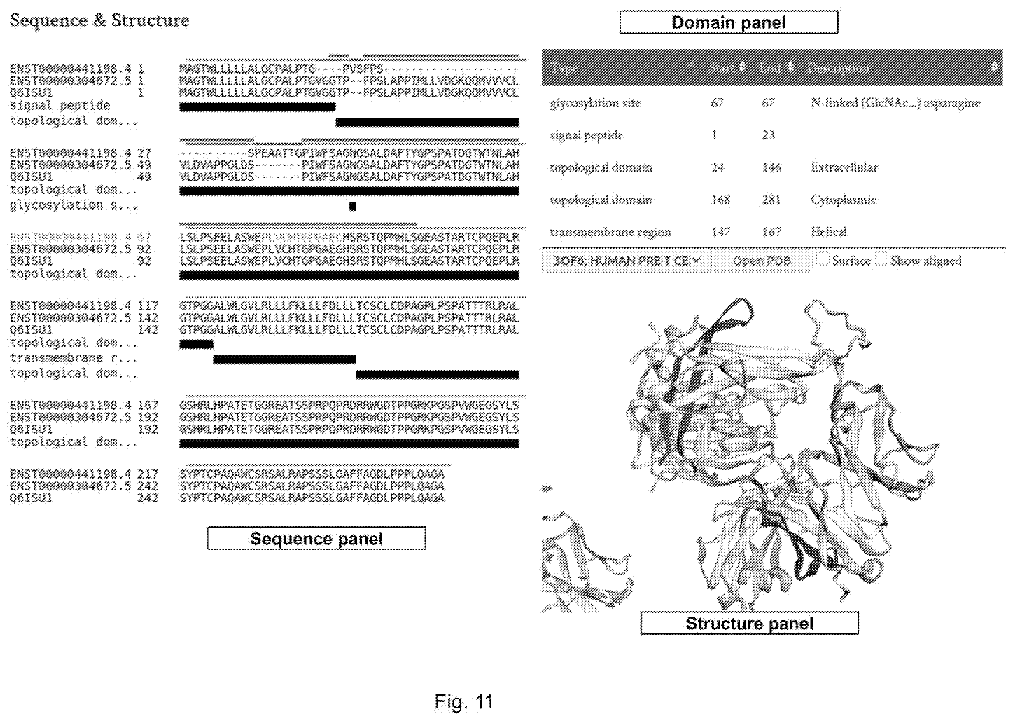

A sequence and structure component generally has three panels: 1) sequence panel, 2) domain panel, 3) and structure panel (see e.g., FIG. 11). The sequence panel shows the sequence alignment between the cancer-specific and the canonical isoforms as well as protein domains. The domain panel shows the protein domains relevant to the isoforms. The structure panel shows known or possible 3D structures of the isoforms.

For every gene in the UniProt database, there is one canonical isoform (referred to as UniProt-canonical isoform). This UniProt-canonical isoform is used to link the three panels:
The start and the end positions of a protein domain in the domain panel are based on a UniProt-canonical isoform. The same UniProt-canonical isoform is mapped to the cancer-specific and the canonical isoforms in the sequence panel, allowing the mapping between isoforms and protein domains.
Cancer-specific and canonical isoforms are mapped to a UniProt-canonical isoform in the sequence panel. The same UniProt-canonical isoform is aligned to the sequence of a 3D protein structure in the structure panel, allowing mapping between isoforms and structures.
The start and the end positions of a protein domain in the domain panel are based on a UniProt-canonical isoform. The same UniProt-canonical isoform is aligned to the sequence of a 3D protein structure in the structure panel, allowing mapping between domains and structures.

All alignment is done using a global alignment method implemented by pairwise2.align.globalms function of BioPython package, with a match score parameter of 5, the mismatch penalty parameter of −4, the gap opening penalty parameter of −5, and the gap extension penalty parameter of −0.1.

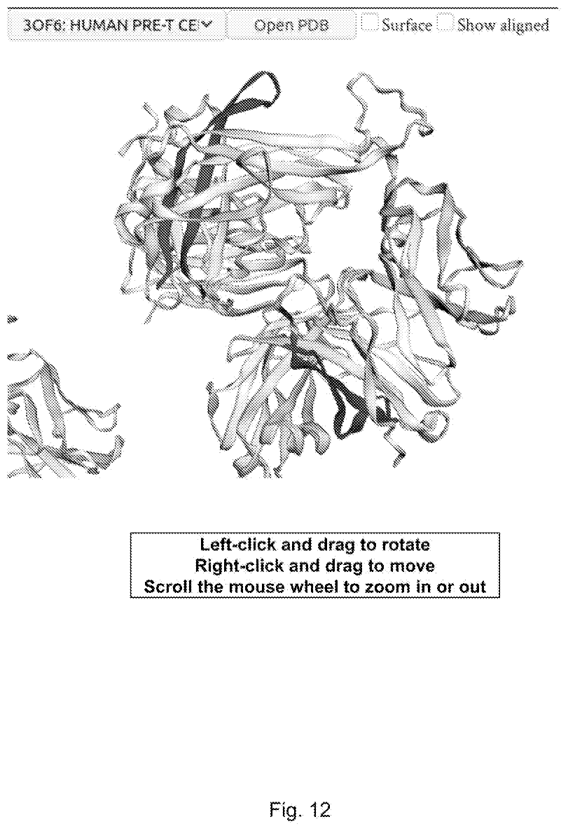

3D protein structures in the structure panel are visualized by 3Dmol.js (World Wide Web Uniform Resource Locator 3dmol.csb.pitt.edu/) library, which enables rotation, zooming in and out, and translation of 3D protein structures with mouse operations (see e.g., FIG. 12).

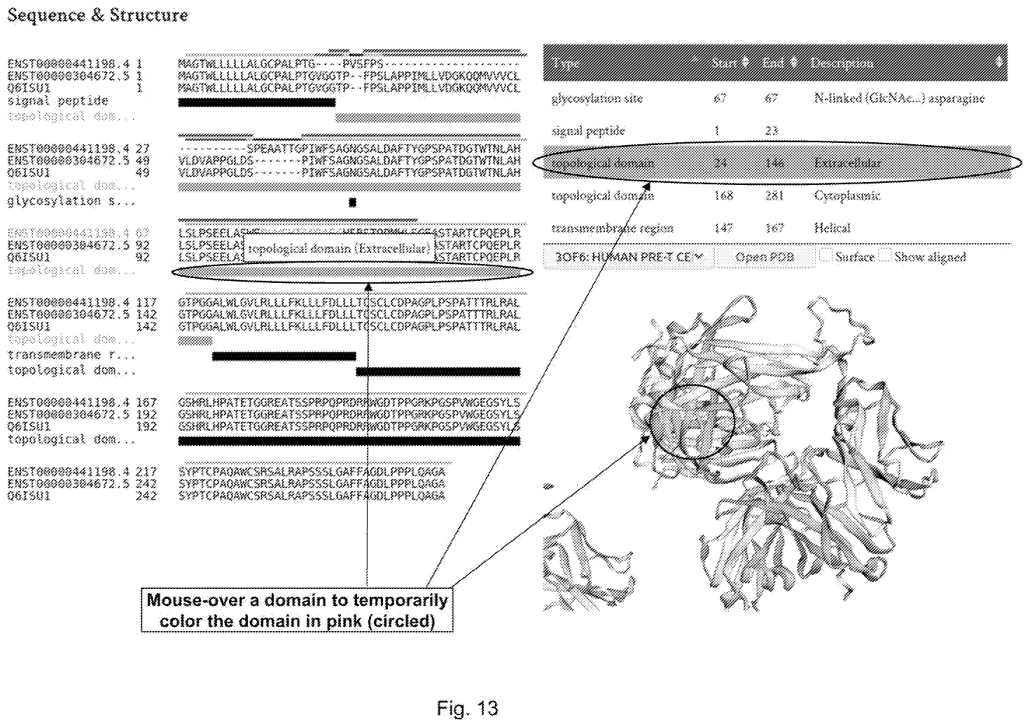

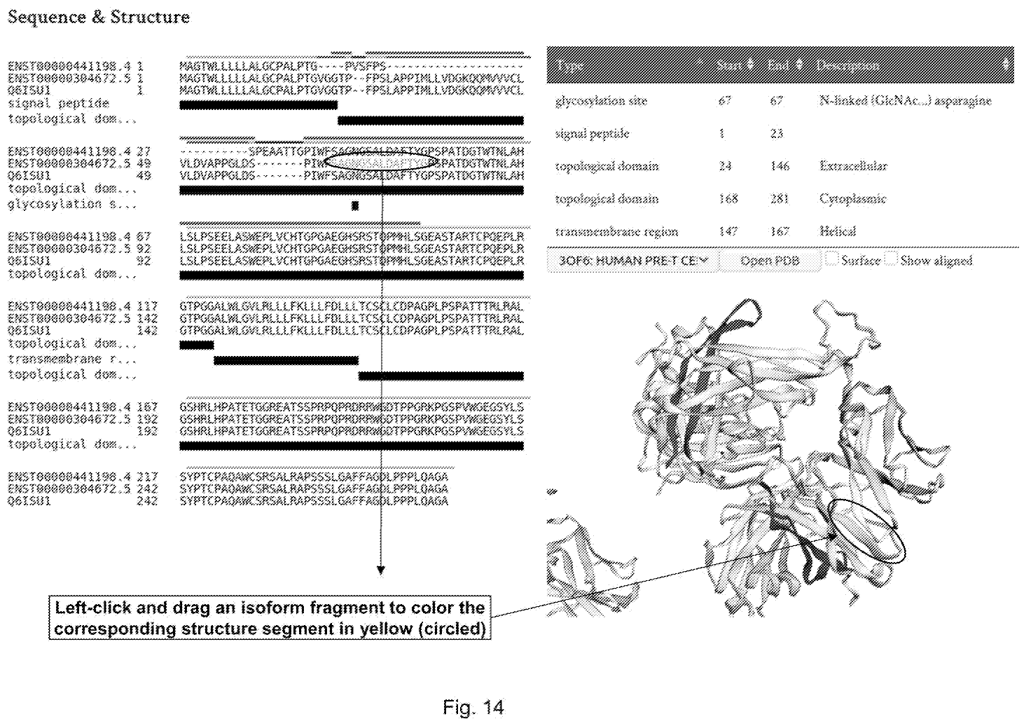

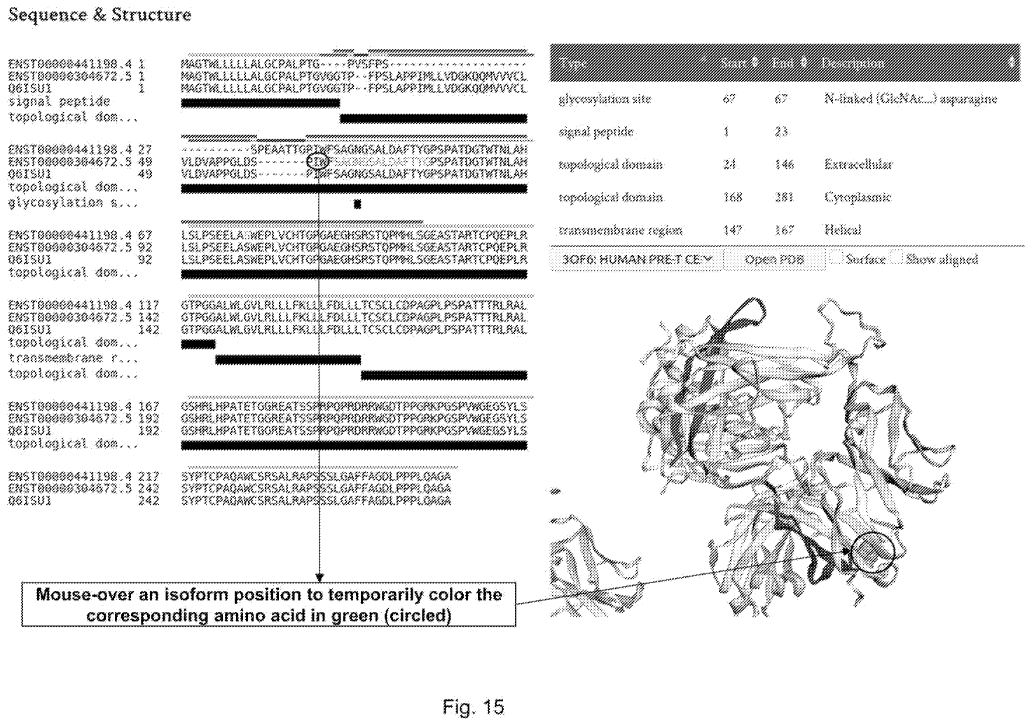

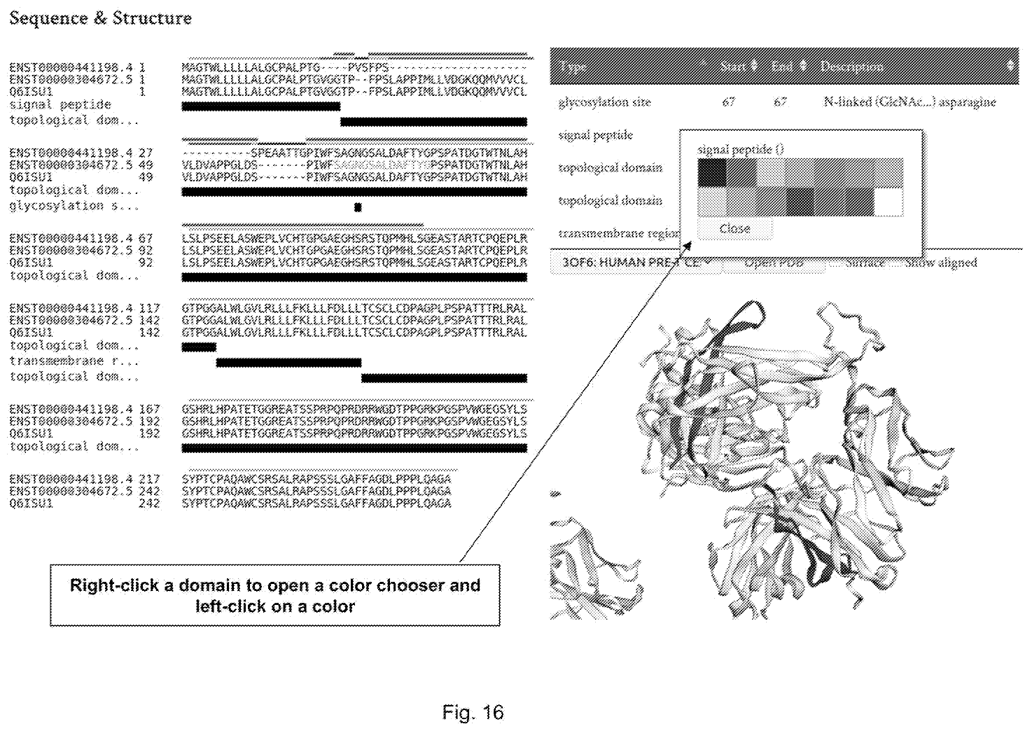

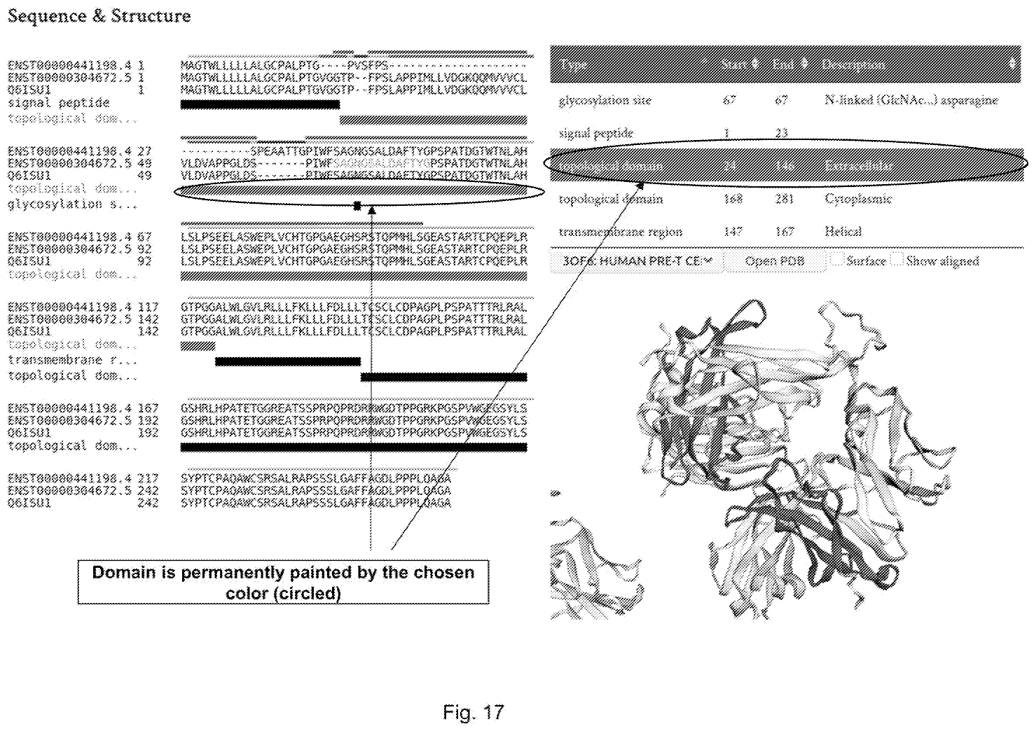

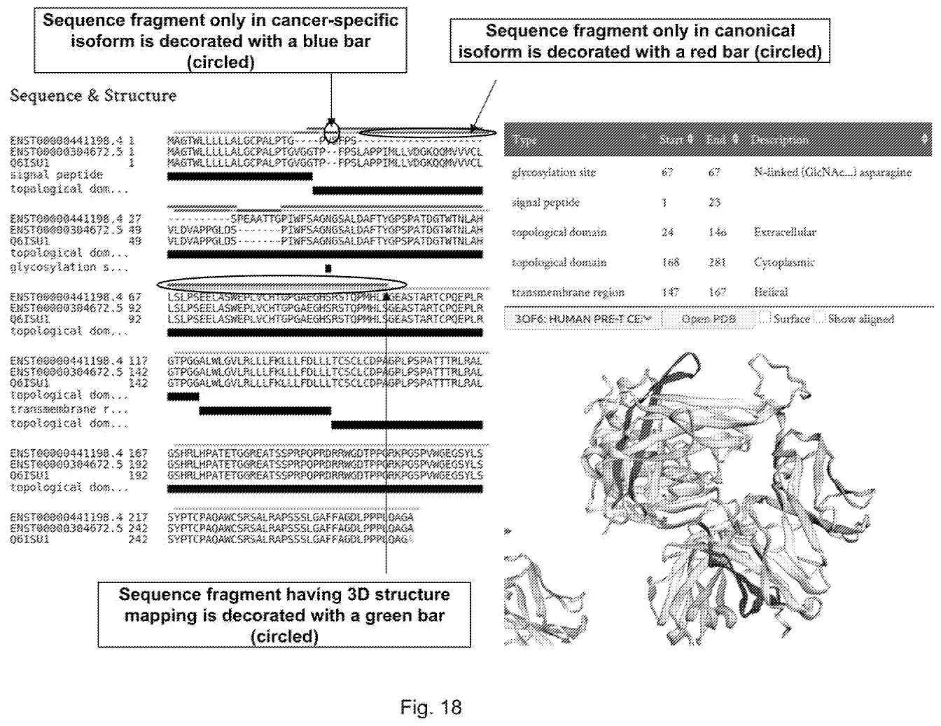

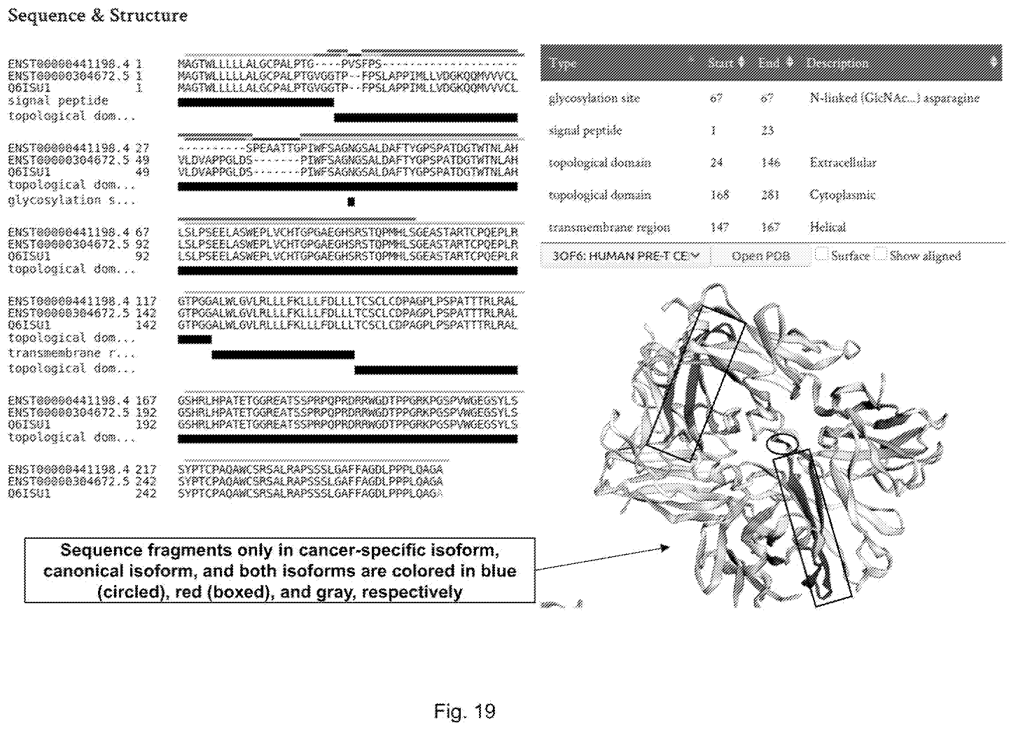

Using the mechanism for mapping among isoform sequences, protein domains, and 3D protein structures, the Sequence & Structure section enable users to do the following:
Mouse-over a protein domain in the sequence panel to temporarily highlight the same protein domain in the domain panel; or mouse-over a protein domain in the domain panel to temporarily highlight the same domain in the sequence panel (see e.g., FIG. 13).
Highlight a fragment of the cancer-specific or the canonical isoform in the sequence panel to highlight the corresponding positions on the 3D protein structure in the structure panel (see e.g., FIG. 14).
Mouse-over a position of the cancer-specific or the canonical isoform in the sequence panel to temporarily highlight the corresponding position on the 3D protein structure in the structure panel (see e.g., FIG. 15).
Right-click a protein domain in the sequence panel or in the domain panel to permanently highlight the same domain in both the sequence and the domain panels with a color of choice (see e.g., FIG. 16 and FIG. 17).
Sequence fragments which exist only in the cancer-specific isoform are decorated with a blue bar in the sequence panel. Sequence fragments which exist only in the canonical isoform are decorated with a red bar in the sequence panel. Sequence fragments which have a mapping 3D protein structure segment are decorated with a green bar in the sequence panel (see e.g., FIG. 18).
3D protein structure segments which have a mapping sequence fragment in the cancer-specific, the canonical, or both isoform are colored red, blue, or gray, respectively, in the structure panel (see e.g., FIG. 19).

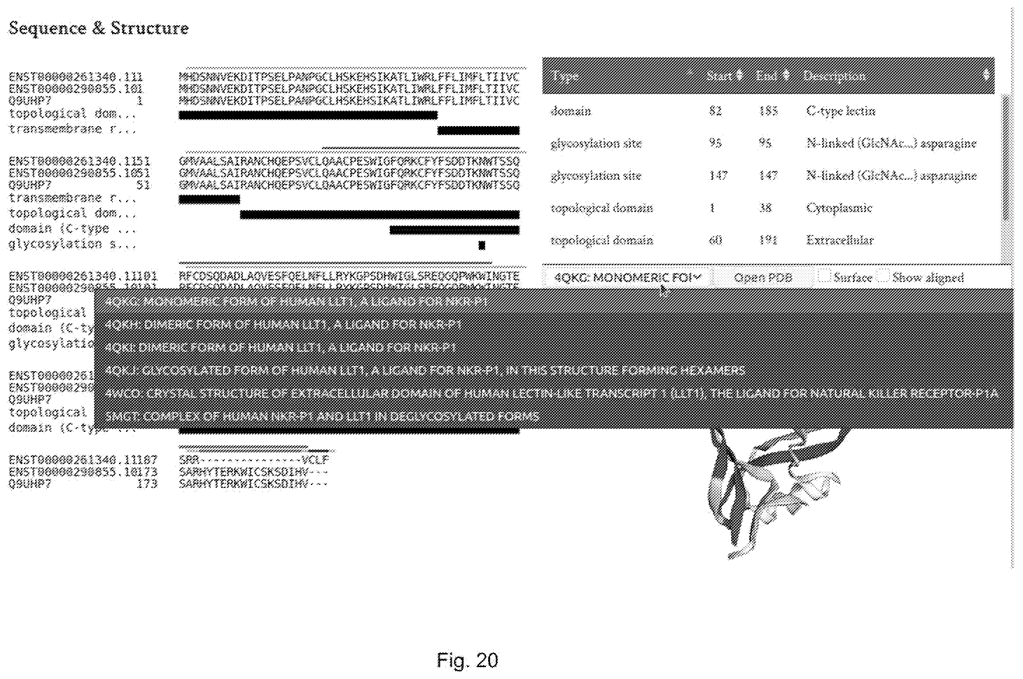

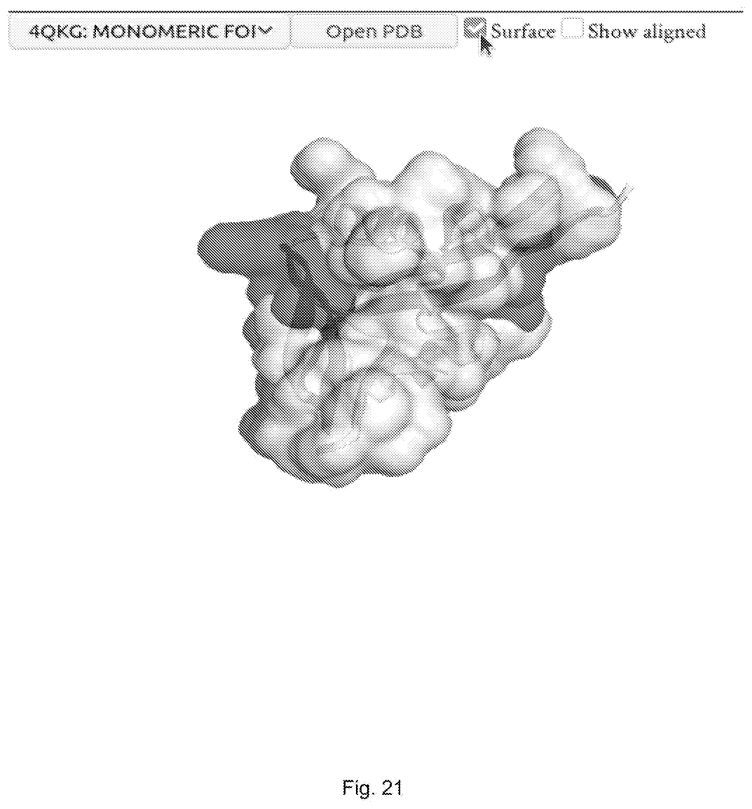

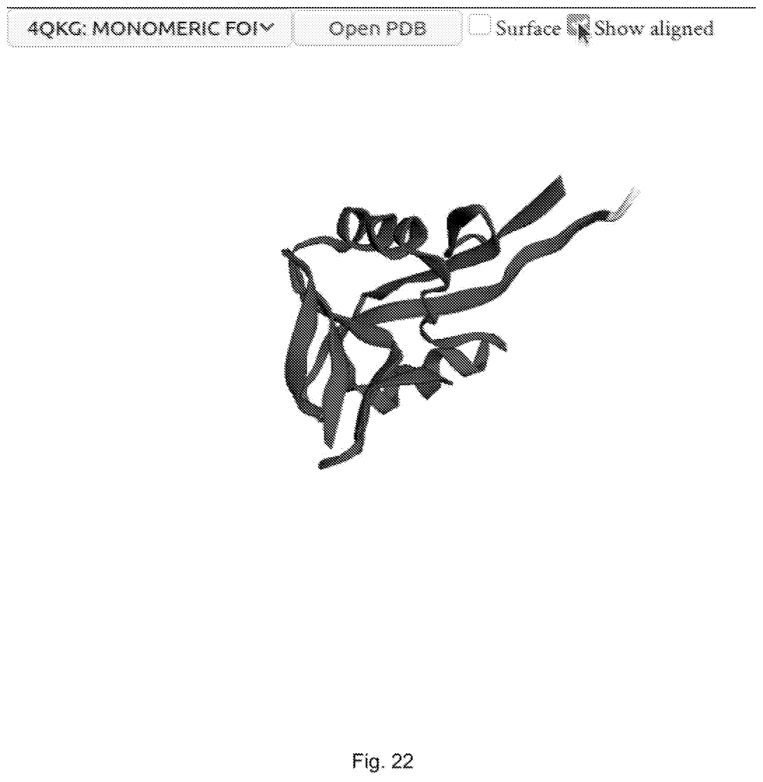

Additionally, the structure panel has the following capabilities:
If there are multiple 3D protein structures are available for the isoforms, a drop-down box will be available for changing the displayed structure (see e.g., FIG. 20).
Solvent-accessible surface area with the red, blue, and gray coloring described above can be turned on or off with a button (see e.g., FIG. 21).
Protein structure segments which are mapped to isoforms can be colored green with a button (see e.g., FIG. 22).

### Images

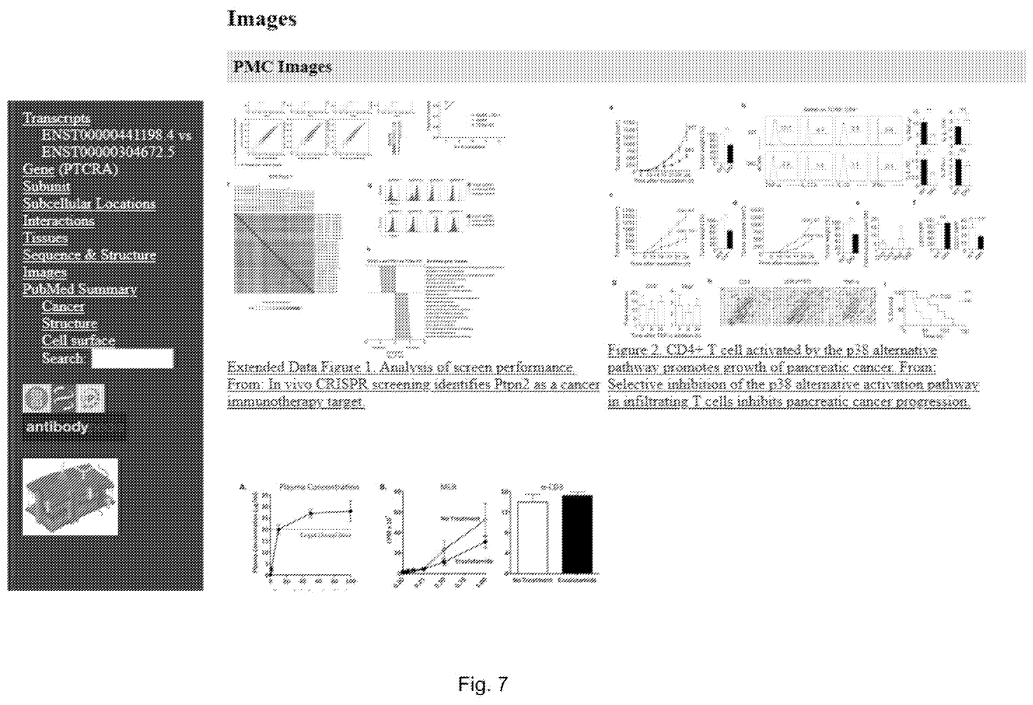

FIG. 7 shows an image search component of the protein analysis tool described herein. Automated searches of Pub Med Central and Bing (or Google) may be performed to find figures and images that include a gene of interest and specific search terms. These automated searches identify background information and include linkouts to the source material. The search terms may be tuned over time by feedback from users.

### Publications

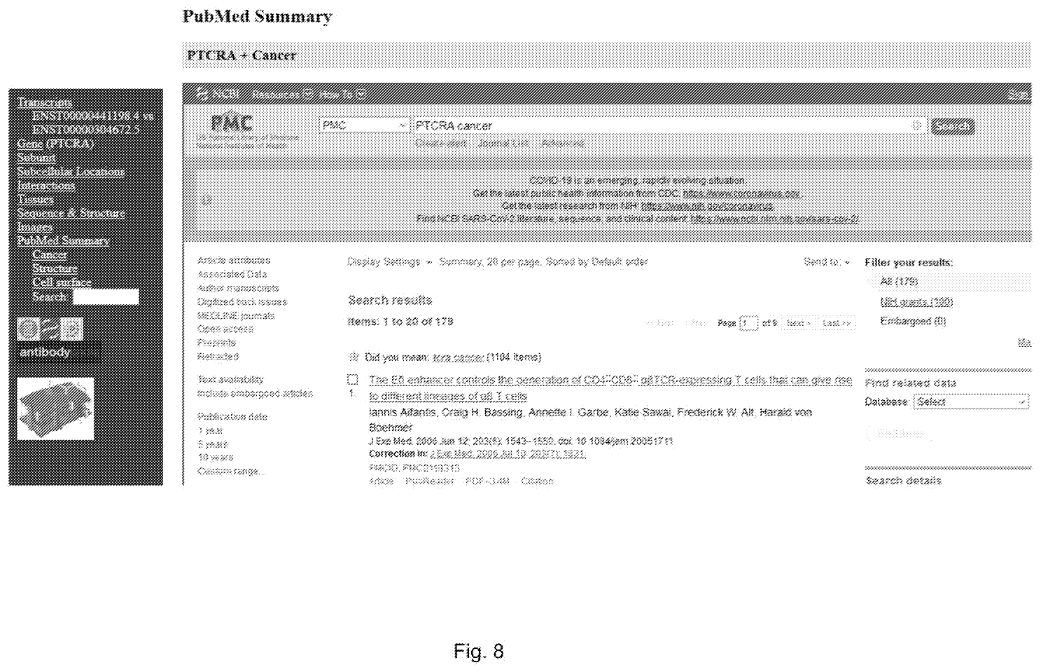

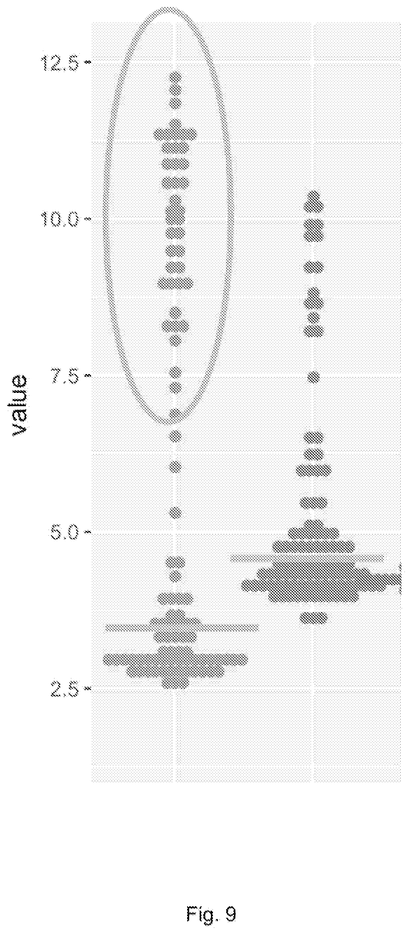

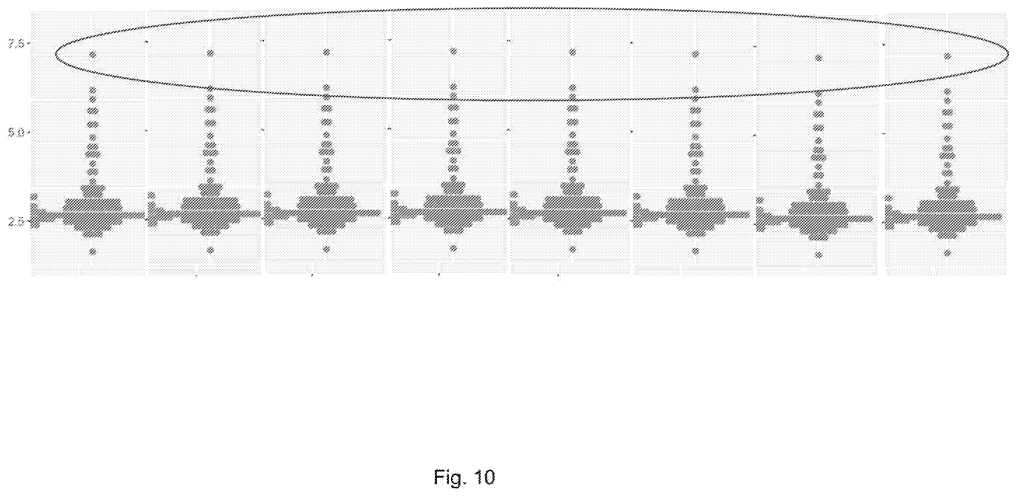

FIG. 8 shows an image search component of the protein analysis tool described herein. Automated searches may be performed for published articles in PubMed. A gene of interest is combined with several search terms to find relevant articles. The top hits including the title and summary are presented. Links to the full article are provided.

### Example 4: Disease-Associated Isoform System Modifications

The disease-associated isoform identification system described herein may include one or more modifications described below.

### Additional Search Option—Median Value

The disease-associated isoform identification system described herein can identify immunotherapy targets by comparing very large sets of tumor data to very large sets of normal data to identify gene expression isoforms that occur in cancer but not in normal tissues. In certain configurations, this comparison is done using highest observed in normal for any normal tissue (top of the whisker plot). In certain configurations, the median normal value is used rather than the maximum normal value (see FIG. 25A), which relaxes this criterion for a search. Median rather than maximum expression in normal tissue generally must be below a threshold level.

### Additional Search Option—Ratio

The ratio method is another modification to the search path. Instead of performing searches using absolute thresholds (normal expression must be below XXX and tumor must be above YYY), a ratio of tumor to normal isoform expression is used (see FIG. 25B). This allows researchers to find targets where there is some expression in normal tissue, but the expression in tumor is much higher. This can result in a target that may have some off target effects (normal tissue targeted), but the very high expression in tumor may result in the tumor being attacked much more often.

### Additional Search Option—Selected Normal Tissues

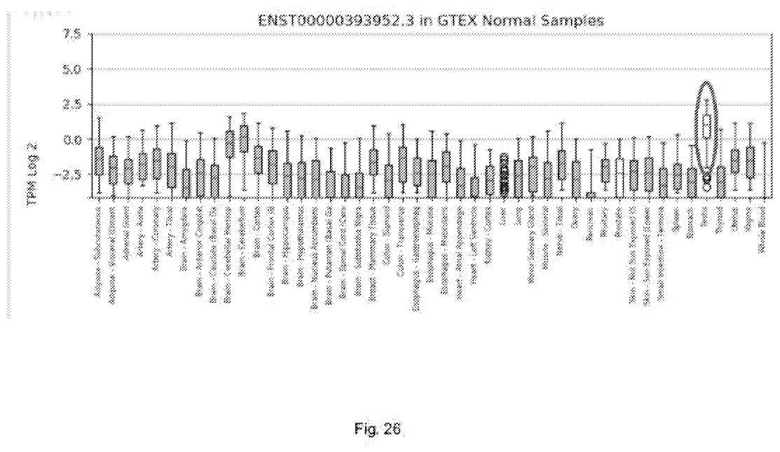

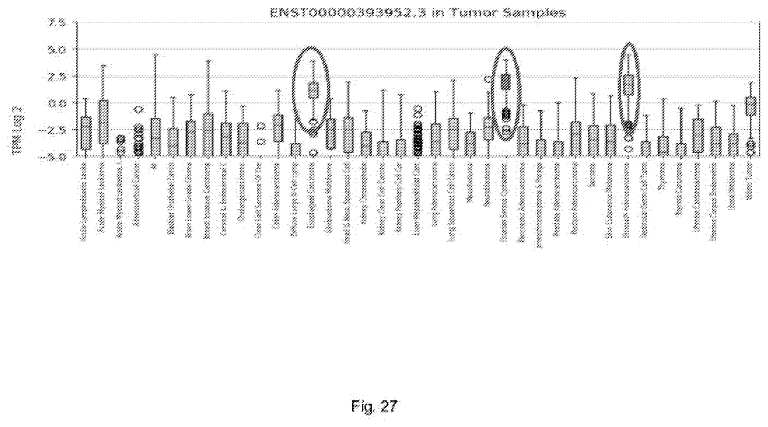

When searching for immunotherapy targets, certain configurations of the disease-associated isoform identification system described herein compare tumor expression to expression in all normal tissue. Targets are identified only if expression is very low in all normal tissues. There are cases, however, where some normal tissues can be ignored. For example, if the therapy is for females and the expression in normal tissue only occurs in males (e.g., in a male-specific tissue like testis), or if the normal tissue is in a relatively unimportant normal tissue. In certain configurations of the disease-associated isoform identification system described herein, researchers can select the normal tissues that are relevant for the therapy being designed. In the example shown in FIG. 25C, the therapy is for ovarian cancer. Accordingly, testis and prostate have been removed from the search. This feature allows researchers to identify new potential targets that may be filtered out using other configurations of the system herein. For example, the target shown in FIG. 26 and FIG. 27 would be excluded using other configurations of the system herein because of high expression in testis. Using a modified normal selection feature, the target is presented and in addition to ovarian cancer, it is also a potential therapy for stomach and esophagus cancer.

### Isoform Merging

Immunotherapy targets can be selected from transcript isoforms identified using the disease-associated isoform identification system described herein. As described herein, isoforms are different versions of mRNA sequences produced by the same gene. The differences are caused by alternative splicing of the mRNA prior to leaving the cell nucleus. To be a good immunotherapy target, the isoform typically needs a section of protein that is different from other isoforms. If the isoform is expressed predominantly in cancer and not normal tissue and has a section of its protein sequence that is unique to the isoform, then it could be a good target. In certain instances, isoforms have a completely unique section of protein. In other instances, an isoform differs from the main isoform of the gene but shares its protein sequence with one or more other isoforms. In this case it may be a viable target but a therapy developed for the isoform will also target the other isoforms with the same protein sequence.

A modified feature of the disease-associated isoform identification system described herein calculates an approximation of tumor and normal expression of all isoforms sharing a target sequence by combining the expression from all the other isoforms that share the target sequence.

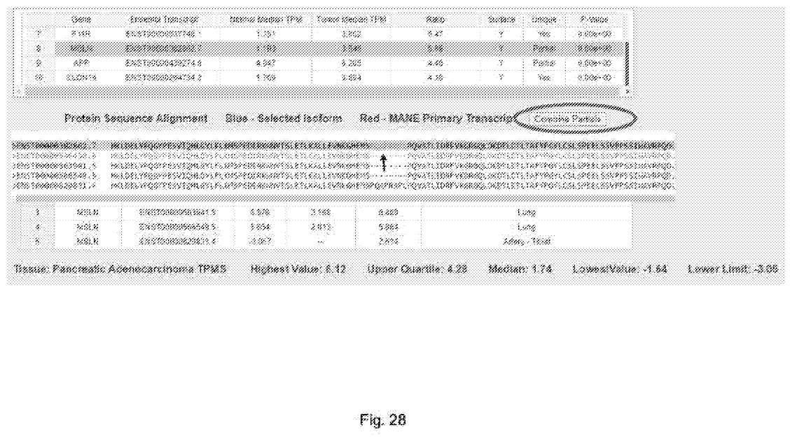

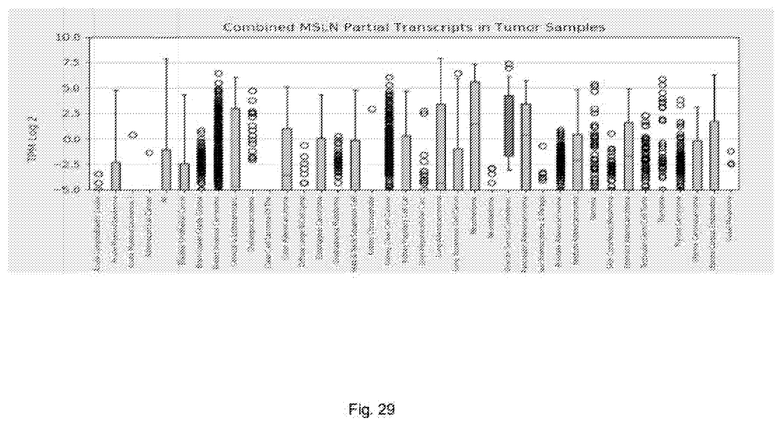

In the example shown in FIG. 28, the isoform ENST00000382862.7 of the MSLN gene is selected. In the Unique column ‘partial’ is indicated. In the protein sequence panel, all the isoforms of MSLN are shown and the arrow indicates the partially unique section of sequence that is the target. It can also be seen that the last isoform, ENST00000620831.4 shares the same sequence so it may also be targeted by the therapy. The ‘Combine Partials’ button merges expression of the two isoforms so researchers can see an approximation of tumor and normal expression of all isoforms sharing the target sequence (see e.g., FIG. 29).

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%).

Certain implementations of the technology are set forth in the claim(s) that follow(s).

1. A computer-implemented method for identifying a transcript of a gene that is expressed at a level in a defined subpopulation of diseased tissue samples higher than a level the transcript is expressed in non-diseased tissue samples, comprising:
(a) receiving user input comprising:
(i) a defined disease selected from a plurality of defined diseases, wherein each of the defined diseases corresponds to diseased tissue samples,
(ii) a defined minimum transcript expression value threshold for diseased tissue samples,
(iii) a defined maximum or defined median transcript expression value threshold for non-diseased tissues, wherein each of the non-diseased tissues corresponds to non-diseased tissue samples, and
(iv) a defined minimum sample sub-population percentage;
(b) identifying in a database comprising one or more tables that relate:
(i) transcript identifiers to genes, wherein:
at least a portion of the transcript identifiers correspond to transcript isoform sets, and each of the transcript isoform sets is encoded by a gene;
(ii) transcript identifiers to corresponding transcript expression values in diseased tissue samples, and
(iii) transcript identifiers to corresponding transcript expression values in non-diseased tissue samples;
one or more transcript identifiers meeting the following criteria (1) and (2):
(1) a corresponding transcript expression value in diseased tissue samples corresponding to the input defined disease of (a)(i) is greater than the input defined minimum transcript expression value threshold of (a)(ii), and
(2) a corresponding transcript expression value in non-diseased tissue is less than the input defined maximum or defined median transcript expression value threshold of (a)(iii),
for a percentage of diseased samples corresponding to the input defined disease of (a)(i) greater than the defined minimum sub-population percentage of (a)(iv); and
(c) outputting a list comprising a transcript identifier for each of one or more transcripts identified in
(b), thereby identifying a transcript of one or more genes that is expressed at a level in a defined subpopulation of diseased tissue samples higher than a level the transcript is expressed in non-diseased tissue samples.

2. The method of claim 1, wherein:
the defined minimum sample sub-population percentage of (a)(iv) is selected from a plurality of defined minimum sample sub-population percentages;
the defined minimum transcript expression value threshold for diseased tissue samples is selected from a plurality of defined minimum transcript expression value thresholds for diseased tissue samples; and/or
the defined maximum transcript expression value threshold for non-diseased tissues is selected from a plurality of defined maximum transcript expression value thresholds for non-diseased tissues, or
the defined median transcript expression value threshold for non-diseased tissues is selected from a plurality of defined median transcript expression value thresholds for non-diseased tissues.

3. The method of claim 2, wherein:
in (b), the one or more tables in the database relate percentages of diseased tissue samples to corresponding transcript identifiers for each defined disease, and
each of the percentages is calculated based on an amount of diseased tissue samples for which the transcript expression values of a corresponding transcript identifier exceed the defined minimum transcript expression value threshold of (a)(ii), wherein each of the percentages is calculated for each defined minimum transcript expression value threshold of the plurality of defined minimum transcript expression value thresholds of (a)(ii).

4. The method of claim 3, wherein each of the percentages equals (i) the number of diseased samples for a defined disease for which transcript expression values for a corresponding transcript identifier exceeds the defined minimum transcript expression value threshold, divided by (ii) total number of diseased samples for the defined disease.

5. The method of claim 4, wherein the list outputted in (c) comprises the percentage of samples exceeding defined minimum transcript expression value threshold of (a)(ii) for each of the transcript identifiers listed.

6. The method of claim 1, comprising:
(d) receiving, after (c), user selection of a transcript identifier outputted in (c); and
(e) outputting the transcript identifier selected in (d) with a transcript identifier for each of one or more transcript isoforms encoded by the gene that encodes the transcript corresponding to the transcript identifier selected in (d).

7. The method of claim 6, comprising, for each transcript identifier outputted, outputting an average transcript expression value in diseased tissue samples corresponding to the input defined disease of (a)(i), and
one or more of:
(i) an average transcript expression value for all non-diseased samples or a subset of non-diseased samples,
(ii) a maximum transcript expression value of non-diseased samples, and
(iii) a non-diseased tissue corresponding to a maximum transcript expression value;
wherein the one or more tables of the database relate the average transcript expression values in diseased tissue samples, the average transcript expression values for all non-diseased samples or a subset of non-diseased samples, the maximum transcript expression values of non-diseased samples, and the non-diseased tissues corresponding to the maximum transcript expression values, to corresponding transcript identifiers; or
one or more of:
(i′) an average transcript expression value for all non-diseased samples or a subset of non-diseased samples,
(ii′) a median transcript expression value of non-diseased samples, and
(iii′) a non-diseased tissue corresponding to a median transcript expression value;
wherein the one or more tables of the database relate the average transcript expression values in diseased tissue samples, the average transcript expression values for all non-diseased samples or a subset of non-diseased samples, the median transcript expression values of non-diseased samples, and the non-diseased tissues corresponding to the median transcript expression values, to corresponding transcript identifiers.

8. The method of claim 6, comprising outputting an alignment of polypeptide linear sequences corresponding to the transcript identifiers outputted in (e), wherein the one or more tables of the database relate polypeptide linear sequences to corresponding transcript identifiers.

9. The method of claim 8, comprising outputting one or more of:
a three-dimensional structure corresponding to one or more of the polypeptide linear sequences, wherein the three-dimensional structure comprises one or more of the following features:
the three-dimensional structure is a user-moveable structure,
the three dimensional structure is annotated with the functional polypeptide domain information, and
the linear polypeptide sequences are mapped to the three-dimensional structure; and
functional polypeptide domain information, wherein the one or more tables of the database relate three-dimensional structure coordinates and functional polypeptide domain information to the polypeptide linear sequences.

10. The method of claim 9, comprising:
receiving a defined portion of a linear polypeptide sequence, and
displaying a portion of a corresponding three-dimensional structure corresponding to the defined portion of the linear polypeptide sequence; or
receiving a defined portion of a three-dimensional structure, and
displaying a portion of a corresponding linear polypeptide sequence corresponding to the defined portion of the three-dimensional structure.

11. The method of claim 1, wherein the one or more tables of the database comprises one or more of the following tables:
a samples table comprising one record for each sample and including phenotype information for each sample;
a tissue table comprising one row for each tissue type and comprising tissues corresponding to the diseased tissue samples and tissues corresponding to the non-diseased tissue samples;
a transcript table relating gene identifiers to corresponding transcript identifiers;
a non-diseased sample statistics table relating transcript expression values to corresponding transcript identifiers for non-diseased samples, and comprising one or more of: average transcript expression value, first quantile of average transcript expression value, third quantile of average transcript expression value, maximum transcript expression value whisker, minimum transcript expression value whisker, and outlier designations for transcripts of non-diseased samples, for corresponding transcript identifiers;
a non-diseased sample statistics by tissue table relating transcript expression values to corresponding transcript identifiers categorized by tissue, and comprising one or more of: average transcript expression value, first quantile of average transcript expression value, third quantile of average transcript expression value, maximum transcript expression value whisker, minimum transcript expression value whisker, outlier designations for transcripts of samples in each tissue, and the tissue having the highest expression value for each transcript identifier, for corresponding transcript identifiers;
a diseased sample statistics by tissue table relating transcript expression values to transcript identifiers categorized by tissue, and comprising one or more of: average transcript expression value, first quantile of average transcript expression value, third quantile of average transcript expression value, maximum transcript expression value whisker, minimum transcript expression value whisker, outlier designations for transcripts of samples in each tissue;
a diseased sample percentage table relating percentages of diseased tissue samples for each defined disease to corresponding transcript identifiers, and
an aligned linear sequences table relating transcript identifiers to corresponding linear polypeptide sequences.

12. The method of claim 11, wherein:
the one or more tables comprise the transcript table,
the transcript table identifies a subset of gene identifiers each corresponding to a gene encoding a cell surface protein,
the user input received in (a) comprises selection of a filter for outputting transcript identifiers corresponding to genes encoding cell surface proteins, and
the one or more transcript identifiers identified in (b) correspond to genes encoding cell surface proteins.

13. The method of claim 11, wherein:
the one or more tables comprise the transcript table,
the transcript table identifies a subset of transcript identifiers each corresponding to a transcript encoding a unique polypeptide comprising an insertion, deletion or substitution of one or more amino acids relative to polypeptides encoded by other transcript isoforms of the same gene;
the user input received in (a) comprises selection of a filter for outputting transcript identifiers corresponding to transcripts encoding unique polypeptides, and
the one or more transcript identifiers identified in (b) correspond to transcripts encoding unique polypeptides.

14. The method of claim 11, wherein:
the one or more tables comprise the transcript table,
the transcript table identifies a subset of transcript identifiers each corresponding to a transcript encoding a unique and/or partially unique polypeptide comprising an insertion, deletion or substitution of one or more amino acids relative to polypeptides encoded by other transcript isoforms of the same gene;
the user input received in (a) comprises selection of a filter for outputting transcript identifiers corresponding to transcripts encoding unique and/or partially unique polypeptides,
the one or more transcript identifiers identified in (b) correspond to transcripts encoding unique and/or partially unique polypeptides, and
the user input further comprises selection of a function for merging the expression values for the transcripts encoding the partially unique polypeptides, thereby generating merged transcript expression values.

15. The method of claim 1, wherein the user input in (a) further comprises selecting a subset of the non-diseased tissues, wherein the defined maximum or defined median transcript expression value threshold for non-diseased tissues in (a)(iii) is determined according to the subset of the non-diseased tissues.

16. A system comprising one or more microprocessors and memory, the memory comprising:
a database comprising one or more tables that relate:
(i) transcript identifiers to genes, wherein:
at least a portion of the transcript identifiers correspond to transcript isoform sets, and each of the transcript isoform sets is encoded by a gene;
(ii) transcript identifiers to corresponding transcript expression values in diseased tissue samples, and
(iii) transcript identifiers to corresponding transcript expression values in non-diseased tissue samples; and
instructions executable by the one or more microprocessors configured to perform the following method:
(a) receiving user input comprising:
(i) a defined disease selected from a plurality of defined diseases, wherein each of the defined diseases corresponds to diseased tissue samples,
(ii) a defined minimum transcript expression value threshold for diseased tissue samples,
(iii) a defined maximum or a defined median transcript expression value threshold for non-diseased tissues, wherein each of the non-diseased tissues corresponds to non-diseased tissue samples, and
(iv) a defined minimum sample sub-population percentage;
(b) identifying in the database one or more transcript identifiers meeting the following criteria (1) and (2):
(1) a corresponding transcript expression value in diseased tissue samples corresponding to the input defined disease of (a)(i) is greater than the input defined minimum transcript expression value threshold of (a)(ii), and
(2) a corresponding transcript expression value in non-diseased tissue is less than the input defined maximum or defined median transcript expression value threshold of (a)(iii),
for a percentage of diseased samples corresponding to the input defined disease of (a)(i) greater than the defined minimum sub-population percentage of (a)(iv); and
(c) outputting a list comprising a transcript identifier for each of one or more transcripts identified in (b).

17. A computer-implemented method for identifying a transcript of a gene that is expressed at a level in a defined subpopulation of diseased tissue samples higher than a level the transcript is expressed in non-diseased tissue samples, comprising:
(a) receiving user input comprising:
(i) a defined disease selected from a plurality of defined diseases, wherein each of the defined diseases corresponds to diseased tissue samples,
(ii) a defined transcript expression ratio threshold, wherein the transcript expression ratio is a ratio of a transcript expression value for diseased tissue samples to a transcript expression value for non-diseased tissues, wherein each of the non-diseased tissues corresponds to non-diseased tissue samples, and
(iii) a defined minimum sample sub-population percentage;
(b) identifying in a database comprising one or more tables that relate:
(i) transcript identifiers to genes, wherein:
at least a portion of the transcript identifiers correspond to transcript isoform sets, and each of the transcript isoform sets is encoded by a gene; and
(ii) transcript identifiers to corresponding transcript expression ratios;
one or more transcript identifiers having a corresponding transcript expression ratio for the input defined disease of (a)(i) that is greater than the input defined transcript expression ratio threshold of (a)(ii),
for a percentage of diseased samples corresponding to the input defined disease of (a)(i) greater than the defined minimum sub-population percentage of (a)(iii); and
(c) outputting a list comprising a transcript identifier for each of one or more transcripts identified in (b), thereby identifying a transcript of one or more genes that is expressed at a level in a defined subpopulation of diseased tissue samples higher than a level the transcript is expressed in non-diseased tissue samples.

18. A system comprising one or more microprocessors and memory, the memory comprising:
a database comprising one or more tables that relate:
(i) transcript identifiers to genes, wherein:
at least a portion of the transcript identifiers correspond to transcript isoform sets, and each of the transcript isoform sets is encoded by a gene;
(ii) transcript identifiers to corresponding transcript expression values in diseased tissue samples, and
(iii) transcript identifiers to corresponding transcript expression values in non-diseased tissue samples; and
instructions executable by the one or more microprocessors configured to perform the following method:
(a) receiving user input comprising:
(i) a defined disease selected from a plurality of defined diseases, wherein each of the defined diseases corresponds to diseased tissue samples,
(ii) a defined transcript expression ratio threshold, wherein the transcript expression ratio is a ratio of a transcript expression value for diseased tissue samples to a transcript expression value for non-diseased tissues, wherein each of the non-diseased tissues corresponds to non-diseased tissue samples, and
(iii) a defined minimum sample sub-population percentage;
(b) identifying in the database one or more transcript identifiers having a corresponding transcript expression ratio for the input defined disease of (a)(i) that is greater than the input defined transcript expression ratio threshold of (a)(ii), for a percentage of diseased samples corresponding to the input defined disease of (a)(i) greater than the defined minimum sub-population percentage of (a)(iii); and
(c) outputting a list comprising a transcript identifier for each of one or more transcripts identified in (b).

19. A computer-implemented method for analyzing a polypeptide comprising:
(a) identifying one or more transcript identifiers in a database comprising one or more tables that relate:
(i) transcript identifiers to genes, wherein:
at least a portion of the transcript identifiers correspond to transcript isoform sets, and each of the transcript isoform sets is encoded by a gene;
(ii) transcript identifiers to corresponding transcript expression values in diseased tissue samples, and
(iii) transcript identifiers to corresponding transcript expression values in non-diseased tissue samples;
(b) receiving user selection of a transcript identifier; and
(c) outputting one or more of:
(i) a three-dimensional structure corresponding to a polypeptide linear sequence corresponding to the selected transcript identifier, and
(ii) functional polypeptide domain information for a polypeptide linear sequence corresponding to the selected transcript identifier, wherein the one or more tables of the database relate three-dimensional structure coordinates and functional polypeptide domain information to the polypeptide linear sequence.

20. A method for generating a database comprising:
(i) relating transcript identifiers to genes, wherein:
at least a portion of the transcript identifiers correspond to transcript isoform sets, and each of the transcript isoform sets is encoded by a gene;
(ii) relating transcript identifiers to corresponding transcript expression values in diseased tissue samples for a plurality of defined diseases; and
(iii) relating percentages of diseased tissue samples for each defined disease to corresponding transcript identifiers, wherein the percentages are based on an amount of diseased tissue samples corresponding to a defined disease for which transcript expression values of a corresponding transcript identifier exceed a defined minimum transcript expression value threshold, or
(iii′) relating percentages of diseased tissue samples for each defined disease to corresponding transcript identifiers, wherein the percentages are based on an amount of diseased tissue samples corresponding to a defined disease for which transcript expression ratios of a corresponding transcript identifier exceed a defined transcript expression ratio threshold.
